(19) United States
(12) Patent Application Publication
(10) Pub. No.: US 2022/0162642 A1
(43) Pub. Date: May 26, 2022

(54) PLASMID SYSTEM

(71) Applicant: FREELINE THERAPEUTICS LIMITED, Stevenage, Hertfordshire (GB)

(72) Inventors: Markus HÖRER, Gilching (DE); Florian SONNTAG, Munich (DE); Renée KOBER, Munich (DE)

(21) Appl. No.: 17/602,941

(22) PCT Filed: Apr. 14, 2020
(86) PCT No.: PCT/GB2020/050947

§ 371 Date: Oct. 11, 2021

(30) Foreign Application Priority Data
Apr. 12, 2019 (EP) 19169121.1
Apr. 12, 2019 (GB) 1905263.8

(51) Int. Cl. C12N 15/86; C07K 14/005

(52) U.S. Cl. CPC C12N 15/86; C07K 14/005; C12N 2750/14151; C12N 2750/14143; C12N 2750/14122

(57) ABSTRACT

The present invention relates to two-plasmid systems, helper plasmids, and/or vector plasmids for producing recombinant AAV (rAAV) vectors. The invention further relates to methods using, or uses of, the two-plasmid systems, helper plasmids and/or vector plasmids of the invention.

### FIELD OF THE INVENTION

The present invention relates to two-plasmid systems, helper plasmids, and/or vector plasmids for producing recombinant AAV (rAAV) vectors. The invention further relates to methods using, or uses of, the two-plasmid systems, helper plasmids and/or vector plasmids of the invention.

### BACKGROUND TO THE INVENTION

Recombinant adeno-associated virus (rAAV) vectors have considerable potential for gene therapy due to their promising safety profile and their ability to transduce many tissues in vivo. However, production is still quite difficult and complex and scale-up of production at an industrial scale has been accomplished only to a limited degree. One reason for this is that rAAV production depends on a co-infection with a helper virus to propagate and establish a productive life-cycle. Infection of cells with a replication-competent helper virus, e.g. an adenovirus, for the production of rAAV has the disadvantage that resulting rAAV stocks are contaminated with helper virus, requiring validated virus removal steps in the down-stream purification process.

For this reason, the use of adenovirus co-infection is commonly avoided by providing the relevant adenoviral helper functions on a plasmid which is co-transfected together with several other plasmids containing the AAV Rep and Cap functions, and the rAAV “vector genome”, i.e. the heterologous nucleic acid comprising the genetic “payload” of the rAAV, flanked by the inverted terminal repeat (ITR) sequences which ensure packaging of the heterologous nucleic acid within the produced viral particles. For example, Emmerling et al (2016) in section 3.1.2 describes four-plasmid systems wherein the Rep, Cap, and adenoviral helper functions are each provided on separate plasmids, together with the vector genome on a fourth plasmid. Plasmid synthesis is a major cost-of-goods driver and the use of four plasmids—all of which must enter the same cell in order for rAAV production to occur in that cell—is disadvantageous from an efficiency and economic perspective.

In section 3.1.3 of Emmerling et al (2016) a two-plasmid system is mentioned, in which one plasmid contains Rep, Cap and vector genome, with the adenoviral functions being provided on the second plasmid. In this regard it must be noted that one major concern of drug regulatory agencies regarding virus-based product safety, whether for oncolytic viruses or gene transfer vectors, is the generation of wild type-like revertants by recombination of the genetic information carried on the starting materials, e.g. in this case upon transfection of the production cells with plasmids collectively carrying this information. Recombination events between the plasmids can result in the generation of so-called replication competent (rc) viruses (in case of AAV, of replication competent AAV (rcAAV) particles). When rep and cap are present on the same plasmid, there is a risk of unacceptable levels of rcAAV being produced by intra- or inter-molecular (depending on which plasmid carries the ITR-heterologous nucleic acid-ITR) recombination.

Such a plasmid arrangement is also economically sub-optimal in the situation where it is desired to switch to a different vector genome (i.e. heterologous nucleic acid of interest; transgene cassette), or to switch to a different capsid serotype (i.e. a different cap gene) which might be desired in the event that a different tissue tropism is sought. In either case, a new (Rep-Cap-vector genome) plasmid will need to be synthesised, the cost of each synthesis being in part a function of the plasmid length, to which the (unchanging) rep gene contributes.

In order to satisfy the current demand of rAAV vector material for clinical trials and market supply, the following goals have yet to be achieved: (a) improved safety and quality profile of the rAAV vectors to meet the regulatory demands on vector quality; (b) improved economics of rAAV manufacture in terms of cost of starting materials for a given manufacturing campaign as well as cost of switching between campaigns; (c) high rAAV production yields; and (d) control over the proportion of produced rAAV particles containing (“full”) or lacking (“empty”) a complete recombinant vector genome.

### SUMMARY OF THE INVENTION

The present invention relates to a two-plasmid system for manufacturing of rAAV, wherein at least some of the adenovirus helper gene functions are combined with AAV rep on one plasmid, and AAV cap is combined with the rAAV vector genome on a second plasmid. The ‘trans-split Rep-Cap’ two-plasmid system is superior over known systems in achieving a combination of improved economics resulting from the simpler and more flexible format, with enhanced safety and quality attributes in addition to other surprising advantages as disclosed herein.

Accordingly, in a first aspect of the invention, there is provided two-plasmid system comprising a helper plasmid and a vector plasmid, wherein the helper plasmid comprises at least one rep gene encoding at least one functional Rep protein and does not comprise a cap gene encoding a functional Cap protein.

In a second aspect of the invention, there is provided a two-plasmid system comprising a helper plasmid and a vector plasmid, wherein the helper plasmid comprises at least one helper virus gene and does not comprise a cap gene encoding a functional Cap protein, and the at least one helper virus gene is comprised in a contiguous stretch of the plasmid having (on one strand) at least 95%, at least 98%, at least 99%, or 100% identity to the full length or to a fragment at least 6000, at least 7000, or at least 8000 nucleotides in length of SEQ ID NO: 4.

Furthermore, the present invention relates to helper plasmids and vector plasmids that are designed for use within a two-plasmid system of the invention.

Accordingly, in a third aspect of the invention, there is provided a helper plasmid comprising at least one rep gene encoding at least one functional Rep protein and at least one helper virus gene, and which does not comprise a cap gene encoding a functional Cap protein.

Similarly, in a fourth aspect of the invention, there is provided a helper plasmid comprising at least one helper virus gene and which does not comprise a cap gene encoding a functional Cap protein, wherein the at least one helper virus gene is comprised in a contiguous stretch of the plasmid having at least 95%, at least 98%, at least 99%, or 100% identity to the full length or to a fragment at least 6000, at least 7000, or at least 8000 nucleotides in length of SEQ ID NO: 4.

Furthermore, in a fifth aspect of the invention, there is provided a vector plasmid comprising:
(a) a cap gene encoding at least one functional Cap protein; or
(b) at least one cap gene promoter, a cloning site operably linked to the cap gene promoter, and an expression cassette flanked on at least one side by an ITR; wherein the vector plasmid does not comprise a rep gene encoding a functional Rep protein and the expression cassette comprises a transgene operably linked to at least one regulatory control element.

In a sixth aspect of the invention, there is provided a host cell comprising the two-plasmid system, the helper plasmid or the vector plasmid of the invention.

The present invention also relates to the use of such two-plasmid systems, helper plasmids and vector plasmids in the manufacture of a rAAV preparation. The plasmids of the invention may be used to obtain rAAV preparations having a low level of rcAAV, having a desired ratio of full to total particles, and/or at a high or desired yield.

In a seventh aspect of the invention, there is provided a use of the two-plasmid system, the helper plasmid or the vector plasmid of the invention for producing a rAAV preparation:
(a) having a low level of replication competent (rcAAV);
(b) having a desired ratio of full to total particles; and/or
(c) at a high or desired yield.

In an eighth aspect of the invention, there is provided a use of the two-plasmid system, the helper plasmid or the vector plasmid of the invention for:
(a) reducing or minimising the level of replication competent (rcAAV) produced during rAAV production;
(b) reducing or minimising the level of pseudo-wild type replication competent AAV (rcAAV) produced during recombinant AAV production;
(c) controlling or maximising the ratio of full to total particles produced during rAAV production; and/or
(d) increasing, optimising or maximising the yield of rAAV produced during rAAV production.

In a ninth aspect of the invention, there is provided a method for producing a rAAV preparation comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

In a tenth aspect of the invention, there is provided a method for reducing or minimising the level of replication competent AAV (rcAAV) produced during rAAV production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

In an eleventh aspect of the invention, there is provided a method for controlling or maximising the ratio of full to total particles produced during rAAV production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

In a twelfth aspect of the invention, there is provided a method for increasing, optimising or maximising the yield of rAAV produced during rAAV production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

In a thirteenth aspect of the invention, there is provided a rAAV preparation obtainable by the methods of the invention.

In a fourteenth aspect of the invention, there is provided a rAAV preparation obtained by the methods of the invention.

In a fifteenth aspect of the invention, there is provided a method for reducing or minimising the level of pseudo-wild type replication competent AAV (rcAAV) produced during rAAV production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

### DESCRIPTION OF THE FIGURES

FIG. 1 provides a schematic of native AAV genome showing overlapping rep and cap (VP1-3) transcripts and position of p5, p19 and p40 promoters. ITR=inverted terminal repeat.

FIG. 2 provides a schematic of helper plasmid constructed as described in Example 1, with inset showing rep genes including p5, p19 and p40 promoters. The crosses through “p40” indicate that these promoters have been rendered non-functional. Hatched area in the rep 52/40 gene indicates the presence of intron sequence which is spliced into rep 52 transcript but spliced out of rep 40 transcript. Ori=bacterial origin of replication. KanR=kanamycin resistance gene. Note the respective plasmid features are not shown to scale.

FIG. 3 provides a schematic of vector plasmid constructed as described in Example 1, with inset showing cap gene and upstream promoter region including p5, p19 and p40 promoters. The crosses through “ATG” and “GTG” indicate that these potential translation initiation codons have been deleted. Ori=bacterial origin of replication. KanR=kanamycin resistance gene. ITR=inverted terminal repeat. Note the respective plasmid features are not shown to scale.

FIG. 4 provides schematics showing vector plasmid embodiments: (A) containing a cap gene, and a multiple cloning site, flanked by ITRs, for cloning-in an expression cassette; (B) containing a cap gene, and a multiple cloning site for cloning-in an ITR-flanked expression cassette; (C) containing an expression cassette, flanked by ITRs, and a multiple cloning site, downstream of a promoter region containing p5, p19 and p40 promoters, for cloning-in a cap gene; (D) containing a multiple cloning site, flanked by ITRs, for cloning-in an expression cassette, and another multiple cloning site, downstream of a promoter region containing p5, p19 and p40 promoters, for cloning-in a cap gene; (E) containing a multiple cloning site for cloning-in an ITR-flanked expression cassette, and another multiple cloning site, downstream of a promoter region containing p5, p19 and p40 promoters, for cloning-in a cap gene.

FIG. 5 provides the results of analysis of rAAV produced and assayed as described in Example 2. (A) Particle titre per ml as measured by anti-capsid ELISA in rAAV produced using the two-plasmid system at varying helper:vector plasmid ratios at constant levels of total plasmid DNA. ‘non-split’=two-plasmid system with Rep and Cap functions on same plasmid, used at plasmid ratio AdV helper-expression cassette:rep-cap 1.6:1. ‘Ctrl’=negative control:helper plasmid transfected with pUC19 instead of vector plasmid. (B) Vector genome (vg) titre per ml as measured by qPCR amplifying a sequence within the promoter of the expression cassette, in the rAAV samples of (A). (C) Ratio of vg (from B) to total particle (from A) ratio, expressed as ‘% full’, i.e. number of vg as a % age of number of particles. Note: 1.0E+12=1.0×10¹². Error bars show standard deviation from triplicate analysis of samples.

FIG. 6 shows rcAAV quantification in different rAAV batches. Three different batches of rAAV containing the same (Factor IX encoding) transgene cassette were analysed for the rcAAV content. Example 3 describes how the batches were analysed.

FIG. 7 shows modulation of yields and full to total particles ratio by plasmid ratio modification. Six different plasmid ratios were tested for the two-plasmid system in rAAV production. The plasmid molar ratios are 3:1, 1.8:1, 1:1.5, 1:2, 1:3 and 1:5. These are a selection of results from the experiments performed for FIG. 5, which have been used to calculate the fold-changes. The fold-changes are relative to the helper.vector plasmid molar ratio 3:1. (A) Virus (vector) genome yields were determined by transgene cassette-specific qPCR. (B) Capsid yields were determined by Capsid-specific ELISA. (C) The full to total particles ratios were calculated based on the qPCR and ELISA results.

FIG. 8 shows (A) virus (vector) genome yields for four different transgenes in the two-plasmid packaging system. Four different transgenes (Factor IX [FIX], alpha-Galactosidase A [GLA], beta-Glucocerebrosidase [GBA] and Factor VIII [FVIII]) were used for rAAV packaging. Two independent packaging experiments were performed for each transgene. Yields were quantified using transgene cassette-specific qPCRs. Different helpervector plasmid ratios have been compared in the context of GBA transgene and FVIII transgene and the results are shown in (B) to (D) for GBA and in (E) to (G) for FVIII. The helper:vector plasmid molar ratios used were 1:0.75, 1:1.5, 1:3 and 1:4.5 as shown. Virus (vector) genome yields were determined by transgene cassette-specific qPCR. Capsid yields were determined by Capsid-specific ELISA. The vector genome to total particle ratios were calculated based on the qPCR and ELISA results. The results are shown as fold-changes relative to the helpervector plasmid molar ratio 1:0.75. (H) shows the virus (vector) genome yields for the four different transgenes (Factor IX [FIX], alpha-Galactosidase A [GLA], beta-Glucocerebrosidase [GBA] and Factor VIII [FVIII]) in the two-plasmid packaging system using the helper:vector plasmid ratios 1:1.8 and 1:3. Yields were quantified using transgene cassette-specific qPCRs. The results are shown as fold-changes relative to the helpervector plasmid molar ratio 1.8:1.

FIG. 9 shows production of rAAV applying different cap serotypes or synthetic cap variants. rAAV was generated applying the identical molar plasmid ratio, five different cap serotypes/synthetic cap variants (the engineered cap gene, AAV-2, AAV-5, AAV-8 or AAV-9) and the same transgene cassette. Virus (vector) genome yields were determined by transgene-cassette specific qPCR.

FIG. 10—sequence listing.

FIG. 11 shows the Southern Blot analysis of DNA isolated from the enrichments for rcAAV. The Southern blot analysis was performed with a cap (A) and rep (B) specific probe. The picture of the corresponding gel with the stained marker bands before blotting was under laid for size correlation of the detected bands on the blot.

“rcAAV”: Replication competent AAV vector preparation containing a 4.7 kb vector genome containing functional rep and cap genes directly loaded on the gel. 1×10⁷ and 1×10⁶ genome copies were loaded as a sensitivity control. Both amounts were loaded at each end of the gel. The 4.7 kb band was detected by both probes as expected. This sample also marks the 4.7 kb wildtype genome length containing functional rep and cap genes.
“Fragment from helper plasmid”: A 4.2 kb plasmid fragment of the helper plasmid P-150 after digestion with BsrGI-HF and NdeI was loaded and served as specificity control as it should only give a signal for the rep probe (and not the cap probe). 1×10⁷ copies of the fragment were loaded.
“Fragment from vector plasmid”: A 3.8 kb plasmid fragment of the vector plasmid P-160 after digestion with ApaLI and PvuI-HF was loaded and served as specificity control as it should only give a signal for the cap probe (and not the rep probe). 1×10⁷ copies of the fragment were loaded.
“Split”: isolated AAV vector genomes after the two rounds of infection (for enrichment of rcAAV) using the rAAV generated using the trans-split two-plasmid system. Also referred to as the “enriched split DNA sample”. The number of copies of cap was estimated using qPCR for the cap sequences as described in Example 10. Approximately 2×10⁷, 4×10⁷ and 6×10⁷ cap copies were loaded on the blots.
“Non-split”: isolated AAV vector genomes after the two rounds of infection (for enrichment of rcAAV) using the rAAV generated using the non-split system. Also referred to as the “enriched non-split DNA sample”. The number of copies of cap was estimated using qPCR for the cap sequences as described in Example 10. Approximately 5×10⁷ cap copies were loaded on the blots.
“rcAAV post-infections”: isolated AAV vector genomes after the two rounds of infection (for enrichment of rcAAV) using replication competent AAV carrying functional rep and cap genes (enrichment positive control). 1×10⁷ genome copies were loaded as a control to demonstrate that rcAAV could be generated using the assay when functional Rep and Cap are present. A clear signal around 4.7 kb was detected.
“M”=Fluorescent High Range DNA ladder (Jena Bioscience): 0.5, 0.6, 0.8, 1.0, 1.5, 2.0, 3.0, 4.0, 5.0, 6.0, 8.0, 10.0 kb

FIG. 12 shows a (1%) agarose gel separation of the amplification products obtained by PCR with the primer pair O-108/109 on the enriched non-split DNA sample (“Non-split”) and the enriched split DNA sample (“Split”). 10 μl of each PCR reaction were loaded per lane.

NTC: no template control (water instead of template DNA)

### Marker (PeqLab): 0.1, 0.2, 0.3, 0.4, 0.5, 0.6, 0.7, 0.8, 0.9, 1.0, 1.2, 1.5, 2.0, 2.5, 3.0, 3.5, 4.0, 5.0, 6.0, 8.0, 10.0 kb

The arrows are indicating the main products obtained: ˜3.5 kb for the non-split sample; weak product of ˜3.5 kb and strong product of ˜2.8 kb for the split sample.

FIG. 13 shows a (1%) agarose gel separation of the amplification products obtained by PCR with primer pairs O-108/109 (repetition of experiment shown in FIG. 12) and O-119/117 on enriched DNA non-split samples (“n.s.”) and enriched DNA split samples (“split”). As a positive control the PCRs were also performed on plasmid P-143 to show the length of the amplification product for a wildtype arranged rep-cap, which is about 3.5 kb for O-108/109 and about 4.1 kb for O-119/117. 10p₁ of each PCR reaction were loaded per lane. The gel on the right side is an enlarged view of the gel containing the PCR products using the O-119/117 primer pair. The gel was exposed for longer in order visualize weak bands. The white arrow indicates the very weak product of O-119/117 on the split sample representing the potentially functional rep-cap containing AAV species which can be easily detected for the non-split sample.

NTC: no template control (water instead of template DNA) of corresponding primer pair.

### Marker (PeqLab): 0.1, 0.2, 0.3, 0.4, 0.5, 0.6, 0.7, 0.8, 0.9, 1.0, 1.2, 1.5, 2.0, 2.5, 3.0, 3.5, 4.0, 5.0, 6.0, 8.0, 10.0 kb

FIG. 14 provides a schematic of regions from the plasmids described in Example 8. The schematic aligns the region from the P-143 plasmid encoding Rep and Cap with the region of the vector plasmid P-160 encoding Cap and the region of the helper plasmid P-150 encoding Rep68, Rep40 and Rep52. There are two stretches of sequences (outlined by the vertical rectangles) which are present in the P-143 plasmid and the P-150 helper plasmid, but which are not present in the P-160 vector plasmid. The dashed lines in the vector plasmid correspond to the sequences which are present in the P-143 plasmid and helper plasmid but are not present in the vector plasmid. The locations where primer pair O-108/109 and primer pair O-117/119 hybridise are indicated. The reverse primers O-109 and O-117 hybridise within the sequence encoding cap, and the forward primers O-119 and O-108 hybridise within the stretch of rep sequence present in the helper plasmid and P-143 plasmid but not present in the vector plasmid. The promoters (p5, p19 and p40) are indicated. The crosses through “p40” indicate that these promoters have been rendered non-functional. The matching pattern of cross-hatching between the plasmids indicate stretches which contain homologous sequences. Note the respective plasmid features are not shown to scale.

FIG. 15 provides a schematic of regions from the P-160 vector plasmid and the P-150 helper plasmid described in Example 8. The matching pattern of cross-hatching between the plasmids indicate stretches which contain homologous sequences. Homologous recombination between the areas of the vector and helper plasmids indicated by the dashed diagonal lines results in the homologous recombination product shown. In the homologous recombination product, amplification using the O-108/109 primer pair results in a product of approximately 2.8 kb, and amplification using the O-119/117 primer pair results in a product of approximately 3.5 kb, as indicated. Each box labelled *** is the portion of the rep locus present in the sequences encoding Rep68/40 and Rep 52/40 in the helper plasmid, but which is missing in the homologous recombination product. The line marked * represents a mutation present in the vector plasmid which is also present in the majority of the homologous recombination products, but not exclusively. The three lines marked ** represent three mutations present in the vector plasmid which are also present in the homologous recombination products. The promoters (p5, p19 and p40) are indicated. The crosses through “p40” indicate that these promoters have been rendered non-functional. Note the respective plasmid features are not shown to scale.

FIG. 16 provides a schematic of regions from the P-160 vector plasmid and the P-150 helper plasmid described in Example 8. The matching pattern of cross-hatching between the plasmids indicate stretches which contain homologous sequences. Homologous recombination between the areas of the vector and helper plasmids indicated by the diagonal lines could result in the homologous recombination product shown. In the homologous recombination product, amplification using the O-108/109 primer pair would result in a product of approximately 3.5 kb, and amplification using the O-119/117 primer pair would result in a product of approximately 4.1 kb, as indicated. Each box labelled *** is the portion of the rep locus present in the sequences encoding Rep68/40 and Rep 52/40 in the helper plasmid and which is missing from the homologous recombination product shown in FIG. 15, but is present in the homologous recombination product of the present FIG. 16. The line marked * represents a mutation present in the vector plasmid. The presence or absence of the mutation in the homologous recombination products could not be determined because the products could not be sequenced because of the low abundance. The three lines marked ** represent three mutations present in the vector plasmid. The presence or absence of the mutations in the homologous recombination products could not be determined because the products could not be sequenced because of the low abundance. The promoters (p5, p19 and p40) are indicated. The crosses through “p40” indicate that these promoters have been rendered non-functional. Note the respective plasmid features are not shown to scale.

### DETAILED DESCRIPTION

### General Definitions

Unless defined otherwise, technical and scientific terms used herein have the same meaning as commonly understood by a person skilled in the art to which this invention belongs.

In general, the term “comprising” is intended to mean including but not limited to. For example, the phrase “a helper plasmid comprising at least one rep gene” should be interpreted to mean that the helper plasmid has at least one rep gene, but the helper plasmid may comprise further components such as further genes.

In some embodiments of the invention, the word “comprising” is replaced with the phrase “consisting of” or the phrase “consisting essentially of”. The term “consisting of” is intended to be limiting. For example, the phrase “a helper plasmid consisting of one rep gene” should be understood to mean that the helper plasmid has one rep gene and no other genetic material. Similarly, the phrase “a helper plasmid consisting essentially of one rep gene” should be understood to mean that the helper plasmid has one rep gene and comprises no additional components that materially affect the function of the helper plasmid. For example a helper plasmid consisting essentially of a rep gene will not contain any other genes but could contain other genetic material such as spacers.

The terms “protein” and “polypeptide” are used interchangeably herein, and are intended to refer to a polymeric chain of amino acids of any length.

For the purpose of this invention, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotides or amino acids at each position are then compared. When a position in the first sequence is occupied by the same amino acid or nucleotide as the corresponding position in the second sequence, then the amino acids or nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence is 95% identical to SEQ ID NO: 1, SEQ ID NO: 1 would be the reference sequence. To assess whether a sequence is at least 80% identical to SEQ ID NO: 1 (an example of a reference sequence), the skilled person would carry out an alignment over the length of SEQ ID NO: 1, and identify how many positions in the test sequence were identical to those of SEQ ID NO: 1. If at least 80% of the positions are identical, the test sequence is at least 80% identical to SEQ ID NO: 1. If the sequence is shorter than SEQ ID NO: 1, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

Herein, the term “plasmid” is intended to refer to a nucleic acid molecule that can replicate independently of a cell chromosome. The term “plasmid” is intended to cover circular nucleic acid molecules and linear nucleic acid molecules. Furthermore, the term “plasmid” is intended to cover bacterial plasmids, but also cosmids, minicircles (Nehlsen, K., Broll S., Bode, J. (2006), Gene Ther. Mol. Biol., 10: 233-244; Kay, M. A., He, C.-Y, Chen, Z.-H. (2010), Nature Biotechnology, 28: 1287-1289) and ministrings (Nafissi N Alqawlaq S. Lee E A, Foldvari M, Spagnuolo P A, Slavcev R A. (2014), Mol Ther Nucleic Acids, 3:e165). Optionally, the plasmid is a circular nucleic acid molecule. Optionally, the plasmid is a nucleic acid molecule that is of bacterial origin.

The term “helper” is not intended to be limiting. Accordingly, a “helper plasmid” is any plasmid that:
(i) comprises at least one rep gene encoding at least one functional Rep protein and does not comprise a cap gene encoding a functional Cap protein; or
(ii) comprises at least one helper virus gene and does not comprise a cap gene encoding a functional Cap protein, and the at least one helper virus gene is comprised in a contiguous stretch of the plasmid having at least 95%, at least 98%, at least 99%, or 100% identity to the full length or to a fragment at least 6000, at least 7000, or at least 8000 nucleotides in length of SEQ ID NO: 4.

The term “vector plasmid” is not intended to be limiting. Accordingly, a “vector plasmid” is any plasmid that:
(i) is suitable for use alongside a helper plasmid in a two-plasmid system of the invention; or
(ii) comprises:
   (a) a cap gene encoding at least one functional Cap protein; or
   (b) at least one cap gene promoter, a cloning site operably linked to the cap gene promoter, and an expression cassette flanked on at least one side by an ITR;
   wherein the vector plasmid does not comprise a rep gene encoding a functional Rep protein and the expression cassette comprises a transgene operably linked to at least one regulatory control element.

The term “nucleic acid molecule” refers to a polymeric form of nucleotides of any length. The nucleotides may be deoxyribonucleotides, ribonucleotides or analogs thereof. Preferably, the plasmid is made up of deoxyribonucleotides or ribonucleotides. Even more preferably, the plasmid is made up of deoxyribonucleotides, i.e. the plasmid is a DNA molecule.

The terms “wild type” and “native” are synonymous and refer to genes present in the genome of a strain/serotype of AAV or adenovirus, or to proteins encoded by genes present in the genome of a strain/serotype of AAV or adenovirus.

AAV Production Assay

In an AAV production assay, the user can determine whether a given “test” plasmid or two-plasmid system is effective to produce rAAV at a similar level to a “reference” plasmid or two-plasmid system as follows.

The user provides a “reference” two-plasmid system that comprises a “reference helper plasmid” and a “reference vector plasmid”.

For example, the user provides a reference helper plasmid comprising wild type Adenovirus 5 helper genes encoding E2A, E4 and VA RNA I and II, i.e. the adenovirus helper genes comprised within SEQ ID NO: 2. Details of the nucleic acid positions in SEQ ID NO: 2 which encode these genes are set out in more detail below under the heading “at least one virus helper gene”. The helper plasmid also comprises a wild type rep gene encoding Rep 40, Rep 52, Rep 68 and Rep 78 and the rep promoters p5, p19 and p40, i.e. the sequences comprised within nucleotides 200-2252 of SEQ ID NO: 1.

The user provides a reference vector plasmid comprising a wild type cap gene operably linked to a wild type cap gene promoter comprising p5, p19 and p40, i.e. the cap gene comprised within SEQ ID NO: 1 (nucleotides 5961-8171 of SEQ ID NO: 1). The vector plasmid further comprises a transgene flanked by two AAV2 ITRs, i.e. the ITRs comprised within nucleotides 1-145 and 4535-4679 of SEQ ID NO: 1.

The user then provides a “test” two-plasmid system comprising a “test helper plasmid” and a “test vector plasmid” that is based on the reference two-plasmid system, but has a single change relating to a characteristic that the users wishes to test. For example, if the user wishes to see whether a given Rep protein was functional, the user could swap out the rep gene of the “reference helper plasmid” and replace it with the test rep protein to provide a “test helper plasmid”.

The user then compares the ability of the reference two-plasmid system and the test two-plasmid system to allow for production of rAAV. To do this, the user can transfect a first set of suitable host cells (such as HEK293T cells which express the E1A/B gene so that the helper plasmid does not need to comprise an E1A/B gene) with the reference two-plasmid system, and a second set of identical host cells with the test two-plasmid system and incubate the host cells for a period of time suitable for the AAV production to occur. The yield of AAV recombinant produced from the reference two-plasmid system and the test two-plasmid system may then be harvested and measured using qPCR to quantify the number of vector genomes. For example, qPCR may be used to determine the number of instances of nucleic acid molecules comprising a component of the vector genome, such as a promoter sequence, that are produced in the host cells transfected with the test two-plasmid system compared to the host cells transfected with the reference two-plasmid system. Alternatively, the comparative yield of particles may be determined, for example by an anti-capsid ELISA.

A suitable AAV production assay is disclosed in Example 2.

A Two-Plasmid System

The present invention provides a two-plasmid system comprising a helper plasmid and a vector plasmid, wherein the helper plasmid comprises at least one rep gene encoding at least one functional Rep protein and does not comprise a cap gene encoding a functional Cap protein.

The present invention also provides a two-plasmid system comprising a helper plasmid and a vector plasmid, wherein the helper plasmid comprises at least one helper virus gene and does not comprise a cap gene encoding a functional Cap protein, and the at least one helper virus gene is comprised in a contiguous stretch of the plasmid having at least 95%, at least 98%, at least 99%, or 100% identity to the full length or to a fragment at least 6000, at least 7000, or at least 8000 nucleotides in length of SEQ ID NO: 4.

Optionally, the helper plasmid and/or the vector plasmid used in the two plasmid system is/are a helper plasmid or vector plasmid of the invention. Optionally, the two-plasmid system comprises a helper plasmid and a vector plasmid of the invention.

The two-plasmid system is useful for producing rAAV. Optionally, the two-plasmid system is suitable for use in producing rAAV. Optionally, the two-plasmid system is for producing rAAV. Optionally, the two-plasmid system is for producing rAAV suitable for use in gene therapy. Optionally, the two-plasmid system is for producing rAAV for use in gene therapy.

The phrase “two-plasmid system” refers to a system that comprises two plasmids, and can be used without the need for additional plasmids to produce rAAV. Optionally, the two-plasmid system can be used to produce rAAV without the need for helper virus such as adenovirus. Optionally, the two-plasmid system can be used to produce rAAV without the need for genetic material originating from a host cell, optionally with the exception of a gene encoding E1A/B. However, the system may comprise additional non-plasmid components. Optionally, the two-plasmid system does not comprise a helper virus. Optionally, the two-plasmid system of the invention comprises all the necessary genetic information for the production of rAAV. For example, the two-plasmid system of the invention may comprise at least one rep gene, at least one cap gene and at least one helper gene. Optionally, the two-plasmid system of the invention comprises all the necessary genetic information required for the production of rAAV suitable for use in gene therapy. For example, the two-plasmid system of the invention may comprise at least one rep gene, at least one cap gene, at least one helper gene and an expression cassette comprising a transgene operably linked to at least one regulatory control element. However, in embodiments the two-plasmid system of the invention may lack a functional cap gene (required for the production of rAAV) and/or an expression cassette comprising a transgene operably linked to at least one regulatory control element (required for the production of rAAV suitable for use in gene therapy).

It is an advantage of the present invention that a cap gene and/or a transgene in the vector plasmid may be exchanged with another in order to treat different genetic disorders. Optionally, therefore, the two-plasmid system of the invention may comprise all the necessary genetic information for the production of rAAV except a functional cap gene, and in such embodiments the two-plasmid system of the invention may comprise a site suitable for cloning in a cap gene. Such a site may comprise a cloning site adjacent to a cap gene promoter. The site suitable for cloning in a cap gene will be present on the vector plasmid. Optionally, the two-plasmid system of the invention comprises all the necessary genetic information for the production of rAAV suitable for use in gene therapy except a functional cap gene and an expression cassette comprising a transgene and a regulatory control element, wherein said two-plasmid system comprises a site suitable for cloning in a cap gene (such as a cap gene promoter and a cloning site operably linked, i.e. adjacent, to the cap gene promoter) and a site suitable for cloning in an expression cassette (such as a cloning site flanked by one or more ITRs). Optionally, the two-plasmid system of the invention comprises all the necessary genetic information for the production of rAAV suitable for use in gene therapy except an expression cassette comprising a transgene and a regulatory control element, wherein said two-plasmid system comprises a site suitable for cloning in an expression cassette (such as a cloning site flanked by one or more ITRs). In such cases the site suitable for cloning in a cap gene and the site suitable for cloning in an expression cassette will be present on the vector plasmid. Optionally, the two-plasmid system of the invention comprises the following components split between the vector plasmid and the helper plasmid:
at least one rep gene encoding at least one functional Rep protein;
at least one helper virus gene;
a cap gene encoding at least one functional capsid protein, or a cap gene promoter and a cloning site operably linked to the cap gene promoter;
at least one ITR; and
an expression cassette comprising a transgene operably linked to at least one regulatory control element, or a site suitable for cloning in an expression cassette flanked on at least one side by (i.e. adjacent to) an ITR.

The vector plasmid may comprise:
(a) a cap gene encoding at least one functional Cap protein; or
(b) at least one cap gene promoter, a cloning site operably linked to the cap gene promoter, and an expression cassette flanked on at least one side by an ITR; wherein the vector plasmid does not comprise a rep gene encoding a functional Rep protein and the expression cassette comprises a transgene operably linked to at least one regulatory control element.

The helper plasmid may comprise:
(a) at least one rep gene encoding at least one functional Rep protein and at least one helper virus gene, and does not comprise a cap gene encoding a functional Cap protein; or
(b) at least one helper virus gene and does not comprise a cap gene encoding a functional Cap protein, and the at least one helper virus gene is comprised in a contiguous stretch of the plasmid having at least 95%, at least 98%, at least 99%, or 100% identity to the full length or to a fragment at least 6000, at least 7000, or at least 8000 nucleotides in length of SEQ ID NO: 4.

Optionally, the ratio of helper plasmid to vector plasmid is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; or between 1:1.5 and 1:3. Optionally, the two-plasmid system comprises a molar excess of vector plasmid compared to helper plasmid. Optionally, the ratio of helper plasmid to vector plasmid is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; between 1:1.5 and 1:3; between 1:2 and 1:4; or around 1:3. Optionally, the ratio of helper plasmid to vector plasmid is between 1:2 and 1:4, or around 1:3. Preferably, the ratio of helper plasmid to vector plasmid is around 1:3.

As set out in more detail below, altering the ratio of helper plasmid to vector plasmid can be used to control the ratio of full to total particles (or proportion of capsids/particles that are full) produced during rAAV production, or to increase the yield of rAAV produced during rAAV production.

By the phrase “ratio of helper plasmid to vector plasmid”, is meant a molar ratio, i.e. the ratio of the number of moles of helper plasmid present to the number of moles of vector plasmid present. A given ratio of helper plasmid to vector plasmid may be provided by simply mixing the helper plasmid and the vector plasmid in an appropriate molar ratio.

A Helper Plasmid

The present invention provides a helper plasmid comprising at least one rep gene encoding at least one functional Rep protein and at least one helper virus gene, and which does not comprise a cap gene encoding a functional Cap protein.

The present invention relates to an improved two-plasmid system that is suitable for producing rAAV. A helper plasmid that comprises at least one rep gene encoding at least one functional Rep protein and does not comprise a cap gene encoding a functional Cap protein may be advantageous for use in such an improved two-plasmid system. Both rep genes and cap genes are required in order to produce rAAV. However, the plasmids and two-plasmid systems of the invention are arranged such that the rep and cap genes are not both present on a single plasmid (‘trans-split’ configuration). Ensuring that the helper plasmid does not comprise both a rep gene and a cap gene is difficult to do, as the native AAV cap and rep genes are overlapping and so separating them whilst maintaining sufficient genetic material to allow for production of the complete protein is difficult. However, splitting the rep genes and the cap genes between a helper plasmid and a vector plasmid is advantageous, as it increases the number of recombination events required to form rcAAV.

The invention also provides a helper plasmid comprising at least one helper virus gene and which does not comprise a cap gene encoding a functional Cap protein, wherein the at least one helper virus gene is comprised on a contiguous stretch of the plasmid having at least 95%, at least 98%, at least 99%, or 100% identity to the full length or to a fragment at least 6000, at least 7000, or at least 8000 nucleotides in length of SEQ ID NO: 4. Optionally the helper plasmid comprises at least one rep gene encoding at least one functional Rep protein.

AAV can only propagate in the presence of a helper virus, which encodes proteins that aid in AAV propagation. However, growing AAV in the presence of a helper virus is not advantageous as helper viruses can be lytic to cells, including host cells used to grow AAV. Furthermore, if helper viruses are used in the production of rAAV products, such as rAAV for use in gene therapy, the helper virus may contaminate the product. As an alternative to co-infecting with helper virus such as adenovirus, the requisite genes of the helper virus can be provided on a transfected plasmid. For host cells expressing the adenoviral E1A/B genes (such as HEK293T cells) the remaining required adenoviral helper genes encode E4, E2A and VA RNA I and II. While these genes are distributed across a long stretch of the adenoviral genome, the present inventors have determined that large stretches of non-coding nucleotides separating the genomic genes can be removed without affecting expression of the genes. The inventors have, therefore, designed a minimal helper gene region, which is SEQ ID NO: 4. Using such a minimal helper gene region in a plasmid for the production of rAAV is advantageous as it allows the user to use a smaller plasmid which is less costly to produce and easier to transfect into the cell.

At Least One Rep Gene

The helper plasmid may comprise at least one rep gene encoding at least one functional Rep protein. AAV comprises a rep gene region which encodes four Rep proteins (Rep 78, Rep 68, Rep 52 and Rep 40). The gene region is under the control of the p5 and p19 promoters. When the p5 promoter is used, a gene that encodes Rep 78 and Rep 68 is transcribed. Rep 78 and Rep 68 are two alternative splice variants (Rep 78 comprises an intron that is excised in Rep 68). Similarly, when the p19 promoter is used, a gene that encodes Rep 52 and Rep 40 is transcribed. Rep 52 and Rep 40 are alternative splice variants (Rep 52 comprises an intron that is excised in Rep 40).

The four Rep proteins are known to be involved in replication and packaging of the viral genome, and are, therefore, useful in rAAV production.

It is not necessary for all four Rep proteins to be present. Optionally, however, the at least one rep gene encodes a large Rep protein (Rep 78 or Rep 68) and a small Rep protein (Rep 52 or Rep 40). Rep 78 can be toxic to cells, and Rep 78 does not need to be present in order for AAV replication to take place. In embodiments where the at least one rep gene does not encode Rep 78, the at least one rep gene preferably does encode Rep 68. Accordingly, the helper plasmid may comprise at least one rep gene encoding:
(a) a functional Rep 52 protein;
(b) a functional Rep 40 protein; and/or
(c) a functional Rep 68 protein.

A “functional” Rep protein is one which allows for production of AAV particles. In particular, Rep 78 or Rep 68 (the large Rep proteins) are believed to be involved in replication of the AAV genome, and Rep 52 and Rep 40 (the small Rep proteins) are believed to be involved in packaging of the AAV genome into a capsid. It is within the abilities of the skilled person to determine whether a given Rep protein is functional. The skilled person merely needs to determine whether the Rep protein supports AAV production using an AAV production assay as described above. In this case, the test two-plasmid system will comprise a helper plasmid that comprises the Rep protein whose “functionality” is to be determined, and otherwise the test two-plasmid system used will be identical to the reference two-plasmid system.

In an embodiment, the at least one rep gene of the helper plasmid encodes a “functional” Rep protein. If the Rep protein supports rAAV production at a level at least 25%, at least 40%, at least 50%, at least 70%, at least 80%, at least 90% or at least 95% of the level supported by the wild type Rep protein, i.e. if the yield of rAAV vector genomes produced is at least 25%, at least 40%, at least 50%, at least 70%, at least 80%, at least 90% or at least 95% of the yield of rAAV vector genomes produced using the reference two-plasmid system. Preferably, the at least one rep gene of the helper plasmid is functional if it supports rAAV production at a level at least 80% of the level supported by the wild type Rep protein.

In general, a Rep protein will only be able to support rAAV production if it is compatible with the ITR(s) surrounding the genome of the AAV to be packaged. Some Rep proteins may only be able to package genomic material (such as an expression cassette) when it is flanked by ITR(s) of the same serotype as the Rep protein. Other Rep proteins are cross-compatible, meaning that they can package genomic material that is flanked by ITR(s) of a different serotype. For example, in the case of a two-plasmid system, it is preferred that the Rep protein is able to support replication and packaging of an expression cassette comprised within the vector plasmid, and such a Rep protein will be compatible with the at least one ITR flanking the expression cassette (i.e. able to replicate and package the expression cassette flanked on at least one side by an ITR).

Optionally, the at least one rep gene comprises a gene encoding a functional Rep 52 protein, at least one gene encoding a functional Rep 40 protein, and a gene encoding a functional Rep 68 protein.

The helper plasmid may comprise two genes encoding a functional Rep 40 protein. In an embodiment, the at least one rep gene comprises two genes encoding a functional Rep 40 protein. For example, the helper plasmid may comprise two rep genes that are separated on the plasmid. The first of the two separate rep genes could encode Rep 68 (for example using the p5 promoter or a different promoter situated near the normal position of the p5 promoter in the rep gene) and Rep 40 (for example using the p19 promoter or a different promoter situated near the normal position of the p19 promoter). The second of the two separate rep genes could encode Rep 52 and Rep 40. Rep 52 and Rep 40 are alternative splice variants.

If the helper plasmid comprises two genes encoding a Rep 40 protein, one of the two genes that encodes a functional Rep 40 protein may comprise an intron. In one embodiment, both genes that encode a functional Rep 40 protein comprise an intron. However, in a preferred embodiment, only one of the genes that encodes a functional Rep 40 protein comprises an intron. For example, if the user wishes to avoid the at least one rep gene encoding Rep 78, the rep gene may be split through partial duplication into two genes. One gene could comprise nucleotides corresponding to the full length native rep gene with the sequence corresponding to the intron removed. Such a gene would encode Rep 68 and Rep 40, but would not encode either Rep 78 or Rep 52, as a portion of each of the Rep 78 and Rep 52 proteins is encoded by the sequence which acts as an intron in the context of rep 40. The second gene could comprise nucleotides corresponding to the region of the native rep gene downstream of the p19 promoter, which would encode Rep 52 (intron spliced in) and Rep 40 (intron spliced out).

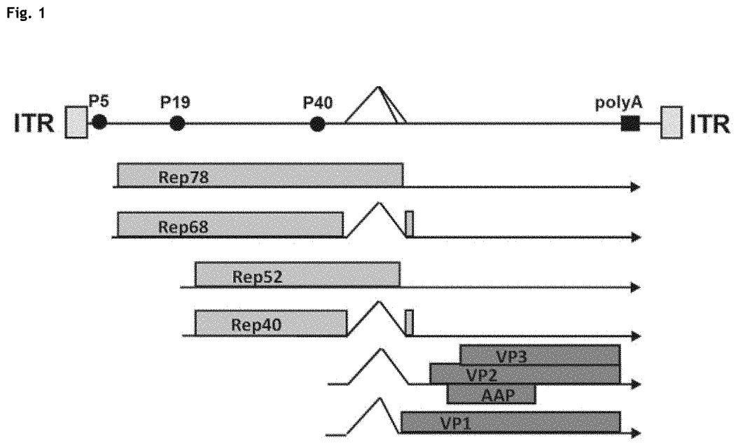

SEQ ID NO: 1 provides the sequence of the genome of wild type AAV2, and nucleotides 321-2252 of SEQ ID NO: 1 encode the four Rep proteins. The full length rep gene (nucleotides 321-2252) encodes all four Rep proteins (Rep 78 and Rep 68 from the p5 promoter and Rep 52 and Rep 40 from the p19 promoter). A shorter stretch of the rep gene downstream of the p19 promoter (nucleotides 993-2252) encodes Rep 52 and Rep 40 only (i.e. this stretch of the rep gene reaches from the end of the p19 promoter to the end of the gene). Nucleotides 1907-2227 of SEQ ID NO: 1 correspond to an intron. Rep 78 and Rep 52 comprise amino acids encoded by the intron, but Rep 68 and Rep 40 are alternative splice variants that do not comprise amino acids encoded by the intron. The relationship between the rep gene and the four Rep proteins is shown in FIG. 1.

Optionally, the two-plasmid system or helper plasmid comprises a gene encoding a functional Rep 52 protein, and the gene encoding a functional Rep 52 protein comprises a nucleic acid sequence having at least 95%, at least 98%, at least 99%, or 100% identity to the full length or a fragment of at least 800, at least 900, at least 1000, or at least 1100 nucleotides in length of nucleotides 993-2186 of SEQ ID NO: 1, or to a corresponding stretch of nucleotides in a different serotype of AAV.

It is within the abilities of the person skilled in the art to determine whether a particular (test) stretch of nucleotides is a “corresponding stretch of nucleotides in a different serotype of” AAV. All that is required is that the person skilled in the art align the test stretch of nucleotides with the genome of the reference serotype (i.e. SEQ ID NO: 1). If the test stretch of nucleotides has greater than 90% identity with a contiguous stretch of nucleotides of the same length in SEQ ID NO: 1, the contiguous stretch is a corresponding stretch of nucleotides in a different serotype of AAV. The same applies in the case of adenovirus sequences (except here the reference serotype is SEQ ID NO: 2).

Optionally, the at least one rep gene comprises a gene encoding a functional Rep 40 protein, and the gene encoding a functional Rep 40 protein comprises a nucleic acid sequence having at least 95%, at least 98%, at least 99%, or 100% identity to the full length or to a fragment of at least 600, at least 700, at least 800, or at least 900 nucleotides in length of a stretch of nucleotides corresponding to nucleotides 993-2252 minus nucleotides 1907-2227 of SEQ ID NO: 1, or to corresponding stretches of nucleotides in a different serotype of AAV. Hence, such a Rep 40-encoding gene has at least the above-specified identity to a notional stretch of nucleotides consisting of nucleotides 993-1906 of SEQ ID NO: 1 immediately juxtaposed with nucleotides 2228-2252 of SEQ ID NO: 1 (5′-[993-1906]-[2228-2252]-3′) or to a notional stretch of nucleotides from a different AAV serotype.

Optionally, the at least one rep gene comprises at least one gene encoding a functional Rep 40 protein, and the gene encoding a functional Rep 40 protein comprises a nucleic acid sequence having at least 95%, at least 98%, at least 99%, or 100% identity to the full length or to a fragment of at least 900, at least 1000, at least 1100, or at least 1200 nucleotides in length of nucleotides 993-2252 of SEQ ID NO: 1, or to a corresponding stretch of nucleotides in a different serotype of AAV.

Optionally, the at least one rep gene comprises a gene encoding a functional Rep 68 protein, and the gene encoding a functional Rep 68 protein comprises a nucleic acid sequence having at least 95%, at least 98%, at least 99%, or 100% identity to the full length or to a fragment of at least 1000, at least 1400, at least 1500, or at least 1600 nucleotides in length of a stretch of nucleotides corresponding to nucleotides 321-2252 minus nucleotides 1907-2227 of SEQ ID NO: 1, or to corresponding stretches of nucleotides in a different serotype of AAV. Hence, such a Rep 68-encoding gene has at least the above-specified identity to a notional stretch of nucleotides consisting of nucleotides 321-1906 of SEQ ID NO: 1 immediately juxtaposed with nucleotides 2228-2252 of SEQ ID NO: 1 (5′-[321-1906]-[2228-2252]-3′), or to a notional stretch of nucleotides from a different AAV serotype.

Since Rep 68 does not comprise any amino acids encoded by the intron (nucleotides 1907-2227), the gene encoding a functional Rep 68 protein does not need to comprise nucleotides corresponding to nucleotides 1907-2227. Indeed, excluding nucleotides 1907-2227 from the at least one rep gene ensures that Rep 78 is not encoded (as Rep 78 comprises amino acids encoded by the intron). Preferably the helper plasmid does not comprise a gene encoding a functional Rep 78 protein.

Optionally, the at least one rep gene comprises a gene encoding functional Rep 68 and Rep 40 proteins, wherein said gene comprises a nucleic acid having at least 95%, at least 98%, at least 99%, or 100% identity to the full length or to a fragment of at least 1400, 1500, 1600 or 1700 nucleotides in length of the following stretches of native AAV2 sequence (SEQ ID NO: 1) positioned in immediate juxtaposition from 5′ to 3′: 200-1906; 2228-2309, or to corresponding juxtaposed stretches of nucleotides from a different serotype of AAV.

Optionally, the at least one rep gene comprises a gene encoding functional Rep 52 and Rep 40 proteins, wherein said gene comprises a nucleic acid having at least 95%, at least 98%, at least 99%, or 100% identity to the full length or to a fragment of at least 1300, 1400, 1500 or 1600 nucleotides in length of the following stretch of native AAV2 sequence (SEQ ID NO: 1): 658-2300, or to a corresponding stretch of nucleotides from a different serotype of AAV.

Optionally, the at least one rep gene comprises a stretch of nucleotides encoding functional Rep 68, Rep 52 and Rep 40 proteins, wherein said stretch comprises a nucleic acid having at least 95%, at least 98%, at least 99%, or 100% identity to the full length or to a fragment of at least 3000, 3200, 3300 or 3400 nucleotides in length of the following stretches of native AAV2 sequence (SEQ ID NO: 1) positioned in immediate juxtaposition from 5′ to 3′: 200-1906; 2228-2309; 658-2300, or to corresponding juxtaposed stretches of nucleotides from a different serotype of AAV.

Optionally, the two-plasmid system or helper plasmid does not comprise a contiguous sequence of at least 1700, at least 1800, or 1866 nucleotides corresponding to a contiguous stretch of nucleotides of equivalent length comprised within nucleotides 321-2186 of SEQ ID NO: 1, or within a corresponding stretch of nucleotides in a different serotype of AAV. The contiguous stretch of nucleotides comprised within nucleotides 321-2186 corresponds to Rep 78.

In some embodiments, the at least one rep gene does not comprise a functional internal p40 promoter. The native rep/cap gene comprises a p40 promoter (D. J. Pereira and N. Muzyczka (1997), J. Virol. 71:1747-1756). The p40 promoter drives expression of the cap gene, but is not required for expression of rep genes. A functional p40 promoter is one that is capable to drive expression of the cap gene.

Whether or not a given p40 promoter is functional may be determined by testing its ability to drive expression of a protein (using an expression assay). For example, user may prepare a “reference” vector comprising a native p40 promoter (such as that of nucleotides 1710-1827 of SEQ ID NO: 1) upstream of a reporter protein such as GFP. The user may then prepare a “test” vector that is identical to the reference vector except that the native p40 promoter is replaced by the p40 promoter whose functionality is to be tested (the “test” p40 promoter). The two vectors may be transfected into suitable host cells, and the host cells incubated under conditions suitable for expression of the reporter protein. The level of reporter protein expressed in the host cells could be measured by, for example, fluorescence spectroscopy. The level of expression from the reference vector (corresponding to the level of expression driven by the native p40 promoter) can then be compared to the level of expression from the test vector (corresponding to the level of expression driven by the test promoter).

A p40 promoter will be considered to be functional if its drives expression at a level at least 40% of the expression driven by the native p40 promoter. Optionally, a functional p40 promoter drives expression at a level at least 60%, at least 70%, at least 80%, at least 90%, or at least 95% of the expression driven by the native p40 promoter. Optionally the at least one rep gene does not comprise a functional internal p40 promoter if it does not comprise a sequence corresponding to the p40 promoter (for example nucleotides 1710-1827 of SEQ ID NO: 1) that drives expression at a level at least 60%, at least 70%, at least 80%, at least 90%, or at least 95% of the expression driven by the native p40 promoter.

P40 promoters lacking a “TATA box” are non-functional. The p40 promoter comprises a TATA box starting at a position corresponding to position 1823 of SEQ ID NO: 1. Thus, the helper plasmid that does not comprise a functional internal p40 promoter may comprise one or more p40 promoters in which the TATA boxes are mutated to render the p40 promoter non-functional. Optionally, the at least one rep gene does not comprise a T nucleotide at a position corresponding to position 1823 of SEQ ID NO: 1. Optionally, the at least one rep gene comprises a C nucleotide at a position corresponding to position 1823 of SEQ ID NO: 1. Optionally, the at least one rep gene does not comprise AAG at positions corresponding to positions 1826-1828 of SEQ ID NO: 1. Optionally, the at least one rep gene comprises CTC at positions corresponding to positions 1826-1828 of SEQ ID NO: 1.

Lack of a Cap Gene

A helper plasmid of the invention does not comprise a cap gene encoding a functional set of Cap proteins.

In native AAV (such as an AAV having a genome as set out in SEQ ID NO: 1), the cap and the rep genes are overlapping. In particular, the p40 promoter, which drives expression of the cap gene is present within the rep gene. Thus, any helper plasmid that encodes Rep proteins will comprise nucleotides from the cap gene. However, a helper plasmid of the invention does not comprise a stretch of nucleotides encoding a functional set of Cap proteins. In particular, a helper plasmid of the invention may not comprise a significant stretch of nucleotide sequence that is exclusively cap gene sequence. The phrase “exclusively cap gene sequence” is intended to refer to gene sequence that encodes a portion of a Cap protein, but does not encode a portion of a Rep protein, for example nucleotides 2253-4410 of SEQ ID NO: 1.

Cap proteins (VP1, VP2 and VP3; see FIG. 1) are proteins that assemble to form a capsid surrounding the viral genome. Thus, a gene encoding a functional set of Cap proteins is one that encodes Cap proteins capable of assembling to encapsidate a viral genome.

A “functional” set of Cap proteins is one which is sufficient for encapsidation of AAV. It is within the abilities of the skilled person to determine whether the product of a cap gene is functional. The skilled person merely needs to determine whether the encoded Cap protein(s) support AAV production using an AAV production assay as described above. In this case, the test two-plasmid system will comprise a vector plasmid that comprises the cap gene which encodes the protein(s) whose “functionality” is to be determined, and otherwise the test two-plasmid system used will be identical to the reference two-plasmid system. The helper plasmid does not comprise a cap gene encoding a functional set of Cap proteins if it does not comprise any genetic material corresponding to a cap gene, or if any cap gene present in the helper plasmid encodes protein(s) which support AAV production at a level below 10% of the level supported by the wild type cap gene product, i.e. if the yield of rAAV produced is less than 10% of the yield of rAAV produced using the reference two-plasmid system.

In general, a functional cap gene is greater than 250 nucleotides in length. Accordingly, the helper plasmid, which does not comprise a cap gene encoding a functional set of Cap proteins, may not comprise a contiguous stretch of exclusively cap gene sequence of more than 250 nucleotides, more than 100 nucleotides, or more than 60 nucleotides. As discussed above, the cap gene and the rep gene in native AAV overlap. Optionally, the helper plasmid does not comprise a contiguous stretch of exclusively cap gene sequence of more than 60 nucleotides. Optionally the helper plasmid does not comprise a cap gene encoding a functional VP1 protein. Optionally, the helper plasmid comprises a portion of cap gene sequence, and the portion of cap gene sequence does not encode a set of functional Cap proteins. The helper plasmid can comprise a portion of cap gene sequence, so long as it does not encode a functional set of Cap proteins. So long as the portion of cap gene sequence is not functional, multiple recombination events would be required to provide a rcAAV.

At Least One Helper Virus Gene

The helper plasmid may comprise at least one helper virus gene. AAV is only able to propagate in the presence of a helper virus. Examples of helper viruses include adenoviruses, and herpes viruses.

However, as set out above, growing AAV in the presence of a helper virus is not advantageous as helper viruses can be lytic to cells, including host cells used to grow AAV. Furthermore, if helper viruses are used in the production of rAAV products, such as gene therapy vectors, the helper virus may contaminate the product. As an alternative to co-infecting with helper virus such as adenovirus, the requisite genes of the helper virus can be provided on a transfected plasmid. For host cells expressing the adenoviral E1A/B genes (such as HEK293T cells) the remaining required helper genes are E4, E2A and VA RNA I and II (a VA nucleic acid). While these genes are distributed across a long stretch of the adenoviral genome, the present inventors have determined that large stretches of non-coding nucleotides separating the genomic genes can be removed without affecting expression of the genes. The inventors have, therefore, designed a minimal helper gene region, which is SEQ ID NO: 4. SEQ ID NO: 4 contains the VA nucleic acid, and the reverse complement (as present in a double stranded plasmid) of an E2A gene and an E4 gene. Using such a minimal helper gene region in a plasmid for the production of rAAV is advantageous as it allows the user to use a smaller plasmid which is less costly to produce and easier to transfect into the cell.

In some embodiments, the helper plasmids of the invention comprise at least one helper virus gene. Preferably, the helper plasmids of the invention comprise sufficient helper genes to allow for AAV replication and packaging. Whether or not a helper plasmid comprises sufficient helper genes to facilitate AAV production can be assessed using an AAV production assay as described above. In this case, the test two-plasmid system will comprise a helper plasmid that comprises the helper genes whose ability to facilitate AAV production is to be tested, and otherwise the test two-plasmid system used will be identical to the reference two-plasmid system. In one embodiment, the helper gene products will be considered to facilitate AAV production if they support rAAV production at a level at least 25%, at least 40%, at least 50%, at least 60%, at least 70%, at least 80%, at least 90% or at least 95% of the level supported by adenoviral helper genes encoding E4, E2A and VA RNA I and II, i.e. if the yield of rAAV produced is at least 25%, at least 40%, at least 50%, at least 60%, at least 70%, at least 80%, at least 90% or at least 95% of the yield of rAAV produced using the reference two-plasmid system. Preferably, the helper gene products will be considered to facilitate AAV production if they support rAAV production at a level at least 70%, at least 80%, at least 90% or at least 95% of the level supported by adenoviral helper genes encoding E4, E2A and VA RNA I and II.

Since the helper plasmids encode the genes required to allow for efficient AAV production, the addition of a helper virus is not required.

Optionally, the at least one helper virus gene is an adenovirus gene. Adenovirus is a virus which is known to aid propagation of AAV (Xiao et al (1998), J. Virol, 72:2224-2232). Optionally, the at least one helper virus gene is an Adenovirus 5 gene or an Adenovirus 2 gene. The genome of Adenovirus 5 is set out in SEQ ID NO: 2, and the genome of Adenovirus 2 is set out in SEQ ID NO: 3. Accordingly, the helper genes may comprise a stretch of nucleotides present in SEQ ID NO: 2 or SEQ ID NO: 3, or a corresponding stretch of nucleotides in another serotype of adenovirus.

The helper genes of adenoviruses encode E1A, E1B, E4, E2A and VA RNA I and II.

E1A is encoded by nucleotides 560-1545 of the Adenovirus 5 genome (for example the genome of SEQ ID NO: 2). Nucleotides 560-1545 contain an intron, from nucleotide 1113 to nucleotide 1228. This intron is not essential, and so an E1A gene comprising nucleotides 560-1112 and 1229-1545 of SEQ ID NO: 2 would encode a functional E1A protein.

E1B is actually two proteins E1B 19K and E 1B 55K, which work together to block apoptosis in adenovirus-infected cells. E1B is encoded by nucleotides 1714-2244 (E1B 19K), and by nucleotides 2019-3509 (E1B 55 K) of the Adenovirus 5 genome (for example the genome of SEQ ID NO: 2).

E4 is encoded by a number of different open reading frames (ORFs) of the Adenovirus 5 genome (for example the genome of SEQ ID NO: 2). E4 ORF 6/7 is encoded by nucleotides 32914-34077, which comprises an intron between nucleotides 33193 and 33903. This intron is not essential, and so an E4 ORF 6/7 comprising nucleotides 32914-33192 and 33904-34077 of SEQ ID NO: 2 is sufficient. E4 34K is encoded by nucleotides 33193-34077 of the Adenovirus 5 genome (for example the genome of SEQ ID NO: 2). E4 ORF 4 is encoded by nucleotides 33998-34342 of the Adenovirus 5 genome (for example the genome of SEQ ID NO: 2). E4 ORF 3 is encoded by nucleotides 34353-34703 of the Adenovirus 5 genome (for example the genome of SEQ ID NO: 2). E4 ORF B is encoded by nucleotides 34700 to 35092 of the Adenovirus 5 genome (for example the genome of SEQ ID NO: 2). E4 ORF 1 is encoded by nucleotides 35140-35526 of the Adenovirus 5 genome (for example the genome of SEQ ID NO: 2).

A functional E4 protein may only comprise amino acids encoded by ORFs 6 and 7, as only the amino acids encoded by ORFs 6 and 7 are required for activity. Optionally, therefore, the functional E4 protein comprises a polypeptide sequence encoded by all or a significant portion of ORFs 6 and 7. Optionally, the functional E4 protein does not comprise polypeptide sequence encoded by all or a portion of ORFs 1-4 and 34K. However, the amino acids encoded by ORFs 1-3 and 34K do improve the activity of the E4 protein, and so in some embodiments the functional E4 protein comprises amino acids encoded by ORFs 1-7.

The E2 (E2A) gene is encoded by nucleotides 22443-24032 of the Adenovirus 5 genome (for example the genome of SEQ ID NO: 2).

The VA RNA I and II is encoded by nucleotides 10589-11044 of the Adenovirus 5 genome (for example the genome of SEQ ID NO: 2).

E1B and E4 are believed to enhance AAV mRNA accumulation, and E2A and VA RNA I and II are believed to enhance AAV mRNA splicing and translation. E1B, E4 and E2A are proteins encoded by genes present in the adenovirus genome, whereas the VA nucleic acid encodes two RNA transcripts known as VA RNA I and VA RNA II. The transcripts themselves are functional in the cell, and are never translated into amino acid sequences. It will be appreciated, therefore, that the VA nucleic acid does not encode a protein, but does “encode” or “correspond to” an RNA, i.e. whilst the term “encode” is used the VA nucleic acid is a non-translated nucleic acid sequence.

Of the five adenovirus genes, it is possible not to include E1A or E 1B in the helper plasmid, as some host cell lines (such as HEK293 cells) express one or more of E1A or E1B constitutively. Optionally, therefore, the helper plasmid does not comprise a gene encoding a functional adenoviral E1A/B protein.

In one embodiment, the at least one helper virus gene comprises:
(a) a VA (viral associated) nucleic acid encoding functional VA RNA I and II;
(b) an E2A gene encoding a functional E2A protein; and/or
(c) an E4 gene encoding a functional E4 protein.

Optionally, the at least one helper virus gene comprises a VA nucleic acid, an E2A gene and an E4 gene.

A “functional” VA RNA I and II, E2A protein or E4 protein is able to facilitate production of AAV. It is within the abilities of the skilled person to determine whether a given VA RNA I and II, E2A protein or E4 protein is functional. The skilled person merely needs to determine whether the VA RNA I and II, E2A protein or E4 protein supports AAV production using an AAV production assay as described above. In this case, the test two-plasmid system will comprise a helper plasmid that comprises the VA RNA I and II, E2A protein or E4 protein whose “functionality” is to be determined, and otherwise the test two-plasmid system used will be identical to the reference two-plasmid system. In an embodiment, the VA RNA I and II, E2A protein or E4 protein will be considered to be “functional” if it supports rAAV production at a level at least 25%, at least 40%, at least 50%, at least 70%, at least 80%, at least 90% or at least 95% of the level supported by the wild type (for example as found in native Adenovirus 5; SEQ ID NO: 2) VA RNA I and II, E2A protein or E4 protein, i.e. if the yield of rAAV produced is at least 25%, at least 40%, at least 50%, at least 70%, at least 80%, at least 90% or at least 95% of the yield of rAAV produced using the reference two-plasmid system. Preferably, the E4 protein will be considered to be “functional” if it supports rAAV production at a level at least 70%, at least 80%, at least 90% or at least 95% of the level supported by the wild type E4 protein.

Optionally, the E4 gene is not located between the VA nucleic acid and the E2A gene, i.e. the sequence of the plasmid is such that E4 gene sequence does not appear in the plasmid between the VA nucleic acid sequence and the E2A gene sequence. Optionally, the E2A gene is located between the VA nucleic acid and the E4 gene.

The helper plasmid is double stranded, and any of the genes comprised within the helper plasmid may be present in a “forward” or “reverse” orientation. For example, the E4 gene may comprise nucleotides 22443-24032 of the Adenovirus 5 genome (for example the genome of SEQ ID NO: 2) or may comprise the reverse complement of nucleotides 22443-24032. Thus, in all instances of the application where reference is made to a plasmid “comprising” a certain nucleic acid sequence, the references should be interpreted as encompassing embodiments where the plasmid comprises the reverse complement of the nucleic acid sequence.

The present inventors have demonstrated that removing all or part of the stretch of nucleotides present at positions 10595-10619 in the adenovirus genome (for example the genome of SEQ ID NO: 2) significantly reduces (by about 50%) the activity of the VA RNA I and II encoded by the VA nucleic acid. Avoiding this reduction in activity is advantageous.

Accordingly, in one embodiment, the VA nucleic acid has an activity level which has at least 75%, at least 80%, at least 90%, at least 95%, or between 95% and 100% of the activity of a wild type VA nucleic acid from Adenovirus 5. Optionally, the activity level of the VA nucleic acid is determined by measuring rAAV yield, for example using the AAV production assay described above. In an embodiment, the VA nucleic acid comprises a contiguous sequence at least 95%, at least 98%, or 100% identical to a stretch of at least 15 nucleotides, at least 20 nucleotides, or 25 nucleotides of nucleotides 10595-10619 of SEQ ID NO: 2, or a corresponding stretch of nucleotides in a different serotype of adenovirus.

Optionally, the E2A gene is operably linked to a promoter which comprises a contiguous sequence at least 96%, at least 98%, or 100% identical to a stretch of at least 60, at least 70, at least 80, or 100 nucleotides of nucleotides 27037-27136 of SEQ ID NO: 2, or a corresponding stretch of nucleotides in a different serotype of adenovirus.

Expression of the E4 gene is driven by a promoter (designated “E4 promoter” herein), corresponding to nucleotides 35585-35848 of SEQ ID NO: 2. Nucleotides corresponding to 35793-35848 of the Adenovirus 5 genome (such as a genome of SEQ ID NO: 2), are required for the promoter to be fully active. Optionally, therefore, the E4 gene is operably linked to an E4 promoter that has at least 50%, at least 70%, or at least 90% of the activity of a wild type promoter from Adenovirus 5. The activity of the E4 promoter may be determined by testing its ability to drive expression of a protein.

Whether or not a given E4 promoter is able to drive E4 gene expression may be determined using an AAV production assay as described above. Specifically, if the E4 promoter can drive E4 gene expression it will facilitate AAV production. In this case, the test two-plasmid system will comprise a vector plasmid that comprises the E4 gene and an E4 gene promoter whose ability to facilitate AAV production is to be tested, and otherwise the test two-plasmid system used will be identical to the reference two-plasmid system. In one embodiment, the E4 promoter will be considered to have at least 25%, at least 40%, at least 50%, at least 70%, or at least 90% of the activity of a wild type E4 promoter from Adenovirus 5 if it supports rAAV production at a level at least 25%, at least 40%, at least 50%, at least 60%, at least 70%, or at least 90% of the level supported by a wild type E4 gene promoter, i.e. if the yield of rAAV produced is at least 50%, at least 70%, or at least 90% of the yield of rAAV produced using the reference two-plasmid system. Preferably, the E4 promoter will be considered to facilitate AAV production if it supports rAAV production at a level at least 70%, at least 80%, at least 90% or at least 95% of the yield of rAAV produced using the reference two-plasmid system.

Optionally, the E4 promoter comprises a sequence of at least 30, at least 40, or 55 nucleotides corresponding to nucleotides 35793-35848 of SEQ ID NO: 2, or a corresponding stretch of nucleotides in a different serotype of adenovirus.

Reducing the Size of the Helper Plasmid

The present inventors have determined that there is a significant amount of non-helper gene nucleic acid sequence between the different helper gene sequences in native Adenovirus 5, and that a substantial portion of this nucleic acid sequence can be removed without significantly impacting the expression level and function of the proteins encoded by the helper gene sequences. Accordingly, the inventors have succeeded in defining a helper gene region with a reduced size, and this can be included in the helper plasmid to reduce its size. Having a reduced size helper gene region is advantageous as it allows the overall size of the helper plasmid to be reduced. Smaller plasmids are cheaper to produce and easier to introduce into host cells.

Accordingly, the helper plasmid may be less than 25000 bp, less than 20000 bp, less than 15000 bp, less than 14500 bp, between 10000 bp and 25000 bp, between 10000 bp and 20000 bp, between 12000 bp and 15000 bp, or around 14021 bp in length.

Similarly, the at least one helper virus gene comprises a VA nucleic acid, an E2A gene and an E4 gene, and the VA nucleic acid, the E2A gene and the E4 gene are comprised within a contiguous stretch of nucleotides on the helper plasmid of fewer than 15000, fewer than 12000, fewer than 10000, fewer than 9000, or fewer than 8500 nucleotides.

An example of a helper gene region with a reduced size is set out in SEQ ID NO: 4. The helper gene region is a contiguous region of the plasmid starting at the first nucleotide encoding a helper gene and ending at the last nucleotide encoding a helper gene. Optionally, the helper gene region comprises a sequence having at least 95%, at least 98%, at least 99%, or 100% identity to the full length or to a fragment at least 6000 nucleotides, at least 7000 nucleotides, or at least 8000 nucleotides in length of SEQ ID NO: 4, i.e. the at least one helper virus gene is comprised on a contiguous stretch of the plasmid having at least 95%, at least 98%, at least 99%, or 100% identity to the full length or to a fragment at least 6000 nucleotides, at least 7000 nucleotides, or at least 8000 nucleotides in length, of SEQ ID NO: 4. Optionally, the helper gene region consists of a sequence having at least 95%, at least 98%, at least 99%, or 100% identity to the full length or to a fragment at least 6000 nucleotides, at least 7000 nucleotides, or at least 8000 nucleotides in length of SEQ ID NO: 4, i.e. the at least one helper virus gene is comprised on a contiguous stretch of the plasmid consisting of a sequence at least 95%, at least 98%, at least 99%, or 100% identity to the full length or to a fragment at least 6000 nucleotides, at least 7000 nucleotides, or at least 8000 nucleotides in length of SEQ ID NO: 4.

The present inventors have identified specific regions of the Adenovirus 5 genome that can be excluded from the helper plasmid.

In one embodiment, the helper plasmid does not comprise a contiguous sequence of at least 2000, at least 2500, at least 3000, or 3427 nucleotides of a contiguous stretch of nucleotides of equivalent length comprised within nucleotides 194-3620 of SEQ ID NO: 2, or a corresponding stretch of nucleotides in a different serotype of adenovirus. In one embodiment, the helper plasmid does not comprise a contiguous sequence of at least 50, at least 60, or 69 nucleotides of a contiguous stretch of nucleotides of equivalent length comprised within nucleotides 4032-4100 of SEQ ID NO: 2, or a corresponding stretch of nucleotides in a different serotype of adenovirus.

In an embodiment, the helper plasmid does not comprise a contiguous sequence of at least 15000, at least 20000, at least 22000, or 22137 nucleotides of a contiguous stretch of nucleotides of equivalent length comprised within nucleotides 10619-32755 of SEQ ID NO: 2, or a corresponding stretch of nucleotides in a different serotype of adenovirus.

In an embodiment, the helper plasmid does not comprise a contiguous sequence of at least 15000, at least 20000, at least 21000, or 21711 nucleotides of a contiguous stretch of nucleotides of equivalent length comprised within nucleotides 11045-32755 of SEQ ID NO: 2, or a corresponding stretch of nucleotides in a different serotype of adenovirus.

Similarly, the inventors have identified regions of the AAV2 genome that do not need to be included in the helper plasmid and may accordingly be excluded in the interests of minimising the size of the helper plasmid. Accordingly, in one embodiment, the helper plasmid does not comprise a contiguous sequence of at least 200, at least 300, at least 350, or 363 nucleotides of a contiguous stretch of nucleotides of equivalent length comprised within nucleotides 4051-4413 of SEQ ID NO: 1, or a corresponding stretch of nucleotides in a different serotype of AAV. Similarly, in one embodiment, the helper plasmid does not comprise a contiguous sequence of at least 400, at least 500, at least 600, or 647 nucleotides of a contiguous stretch of nucleotides of equivalent length comprised within nucleotides 2301-2947 of SEQ ID NO: 1, or a corresponding stretch of nucleotides in a different serotype of AAV.

Artificial Rep Binding Sites

Rep proteins bind to a rep binding site (RBS), which is a nucleic acid sequence having a consensus sequence of GCTCGCTCGCTCGCTC (SEQ ID NO: 6) (McCarty, D. M. et al. (1994), J. Virol., 68(8): 4988-4997)). Accordingly, a rep binding site is a nucleic acid sequence having homology to GCTCGCTCGCTCGCTC (SEQ ID NO: 6). Determining whether a nucleic acid sequence (test nucleic acid sequence) comprises a rep binding site, for example, determining whether a nucleic acid sequence comprises a sequence having homology to GCTCGCTCGCTCGCTC (SEQ ID NO: 6), can be carried out using an electrophoretic mobility shift assay (EMSA). The test nucleic acid sequence is applied to a first well of a gel suitable for use in electrophoresis, and a mixture of the test nucleic acid sequence and Rep68/Rep78 proteins is applied to a second well of the gel. If the test nucleic acid sequence comprises a rep binding site, this will cause a shift in the mobility of the nucleic acid sequence in the well comprising the Rep68/Rep78 proteins compared to the well that lacked the Rep68/Rep78 proteins.

Binding of Rep proteins to aberrant RBSs might result in Rep-mediated non-homologous recombination between different sequences comprising RBSs, resulting in undesired fusion sequences. For example, Rep proteins could bind the RBS in AAV ITRs and any RBS present on a bacterial plasmid backbone resulting in the fusion of plasmid backbone sequences to an ITR. This could result in enhanced replication and packaging of such undesired fusion sequences.

Accordingly, it is preferred that the only RBSs present in the plasmids of the invention correspond to RBSs present in wild type AAV (allowing the Rep proteins to carry out their normal function). However, when a plasmid is constructed it is possible that an RBS will be present, either in the plasmid backbone or elsewhere in the plasmid in non-AAV-derived sequences, and such sequences are considered to be “artificial” or “aberrant” RBSs, and should be avoided/removed.

A common source of artificial RBSs in plasmids is from the plasmid backbone. The plasmid backbone is the bacterial sequence needed to amplify the plasmids in bacterial host cells. The plasmid backbone may be the region of the plasmid that is not derived from adenovirus or AAV or is not situated between two AAV-derived ITRs. Optionally, the vector plasmid backbone encompasses any nucleotides except the cap gene, a promoter (region) operably linked to the cap gene, ITRs and the expression cassette. Optionally, the helper plasmid backbone encompasses any nucleotides except the at least one helper gene and associated adenovirus-derived regulatory elements, the at least one rep gene and associated AAV-derived regulatory elements, and one or more promoters operably linked to the at least one rep gene. In one embodiment, the helper plasmid and/or the vector plasmid comprises a backbone, and the plasmid backbone does not comprise an artificial RBS. Optionally, the helper plasmid and the vector plasmid each comprise a backbone, and neither plasmid backbone comprises an artificial RBS.

Accordingly, in one embodiment, the helper plasmid and/or the vector plasmid does not comprise an artificial RBS. Preferably, the helper plasmid and the vector plasmid do not comprise an artificial RBS. Optionally, there are no RBSs in the vector plasmid and/or the helper plasmid, except within any ITR(s), p5 promoter(s) and p19 promoter(s).

Cap Gene

The vector plasmid may comprise a cap gene. The cap gene encodes a functional Cap protein. The cap gene may encode a functional set of Cap proteins. AAV generally comprise three Cap proteins, VP1, VP2 and VP3. These three proteins form a capsid into which the AAV genome is inserted, and allow the transfer of the AAV genome into a host cell. All of VP1, VP2 and VP3 are encoded in native AAV by a single gene, the cap gene. The amino acid sequence of VP1 comprises the sequence of VP2. The portion of VP1 which does not form part of VP2 is referred to as VP1 unique or VP1U. The amino acid sequence of VP2 comprises the sequence of VP3. The portion of VP2 which does not form part of VP3 is referred to as VP2unique or VP2U.

A “functional” set of Cap proteins is one which allows for encapsidation of AAV. As discussed above, it is within the abilities of the skilled person to determine whether a given Cap protein is or a set of Cap proteins are functional. The skilled person merely needs to determine whether the encoded Cap protein(s) support AAV production using an AAV production assay as described above. In this case, the test two-plasmid system will comprise a vector plasmid that comprises the cap gene which encodes the Cap protein(s) whose “functionality” is to be determined, and otherwise the test two-plasmid system used will be identical to the reference two-plasmid system. The Cap protein(s) will be considered to be “functional” if it/they support(s) rAAV production at a level at least 25%, at least 40%, at least 50%, at least 70%, at least 80%, at least 90% or at least 95% of the level supported by the wild type cap gene product, i.e. if the yield of rAAV produced is at least 25%, at least 40%, at least 50%, at least 70%, at least 80%, at least 90% or at least 95% of the yield of rAAV produced using the reference two-plasmid system. Preferably, the Cap protein(s) will be considered to be “functional” if it/they support(s) rAAV production at a level at least 70%, at least 80%, at least 90% or at least 95% of the level supported by the wild type cap gene product.

Optionally, VP2 and/or VP3 proteins are “functional” if an AAV comprising the VP2 and/or the VP3 proteins is able to transduce Huh7 cells at a level at least 25%, at least 40%, at least 50%, at least 70%, at least 80%, at least 90% or at least 95% of that of an equivalent AAV comprising a wild type VP2 and/or VP3 protein. The ability of an AAV particle to transduce Huh7 cells can be tested by adding a reporter protein such as green fluorescent protein (GFP) to the AAV particle, mixing the AAV particle with Huh7 cells, and measuring the fluorescence produced.

Optionally, the vector plasmid comprises a cap gene that encodes a VP1, a VP2 and/or a VP3 protein. Optionally, the VP1, VP2 and VP3 proteins are expressed from more than one cap gene. Optionally, the vector plasmid comprises a cap gene that encodes a VP1, a VP2 and a VP3 protein. Optionally the vector plasmid comprises a cap gene encoding a functional VP1, i.e. a VP1 protein capable of assembling with other Cap proteins to encapsidate a viral genome.

Different serotypes of AAV have Cap proteins having different amino acid sequences. A cap gene encoding any (set of) Cap protein(s) is suitable for use in connection with the present invention. The Cap protein can be a native Cap protein expressed in AAV of a certain serotype. Alternatively, the Cap protein can be a non-natural, for example an engineered, Cap protein, which is designed to comprise a sequence different to that of a native AAV Cap protein. Genes encoding non-natural Cap proteins are particularly advantageous, as in the context of gene therapy applications it is possible that fewer potential patients have levels of antibodies that prevent transduction by AAV comprising non-natural Cap proteins, relative to native capsids.

Optionally, the cap gene encodes a Cap protein from a serotype selected from the group consisting of serotypes 1, 2, 3A, 3B, 4, 5, 6, 7, 8, 9, 10, 11, 12, or 13. Optionally, the cap gene encodes a Cap protein from a serotype selected from the group consisting of serotypes 2, 5, 8, and 9. Optionally, the cap gene encodes a Cap protein selected from the group consisting of LK03, rh74, rh10 and Mut C (WO 2016/181123; WO 2013/029030; WO 2017/096164). Optionally, the cap gene encodes a Cap protein selected from the group of AAV serotypes consisting of serotypes 2, 5, 8 or 9, and Mut C (SEQ ID NO: 3 from WO 2016/181123). Optionally, the cap gene encodes the Cap protein Mut C (SEQ ID NO: 3 from WO 2016/181123).

Cap Gene Promoter

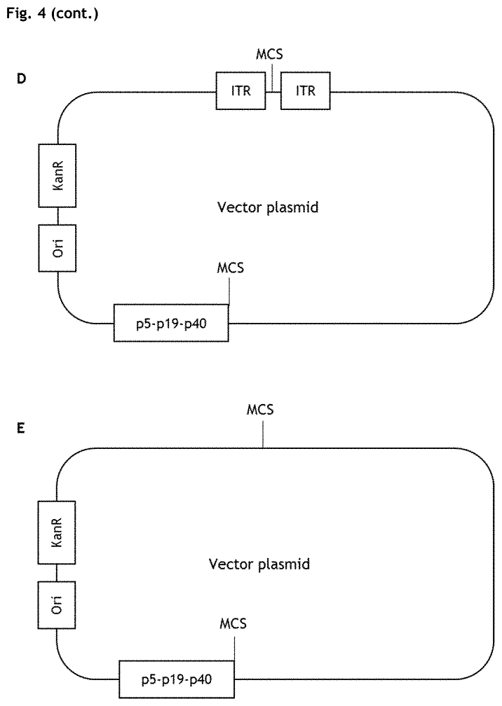

Optionally, the vector plasmid comprises a cap gene promoter. The cap gene promoter may be operably linked to a cap gene. Alternatively, the vector plasmid may not comprise a cap gene, but may comprise a cloning site operably linked (i.e. in close juxtaposition: 5′-[cap gene promoter]-[cloning site]-3′) to the cap gene promoter. The cloning site may be a multiple cloning site (MCS, or polylinker; see for example FIG. 4C-E). The user may wish to have the option to add a specific cap gene for a specific application. For example, if the vector plasmid is to be used to produce AAV for use in gene therapy, the user may wish the vector plasmid to lack a cap gene, but comprise a cloning site to allow a specific cap gene to be cloned in for a specific application. The vector plasmid could be used in connection with any transgene (in an expression cassette), and the user may find that for certain transgenes, encapsidation of such cassettes into capsids having properties such as liver tropism is advantageous, whereas for other transgenes capsids having different tropisms are advantageous. By designing a vector plasmid that comprises a cap gene promoter linked to a cloning site, the user can readily ‘plug in’ an appropriate cap gene for a specific application (such as use of a specific transgene).

Optionally, the vector plasmid comprises an at least one cap gene promoter, which is a native cap gene promoter.

The native cap gene (i.e. the cap gene of a wild type AAV) is operably linked to a p40 promoter, a p5 promoter and a p19 promoter. Optionally, the at least one cap gene promoter comprises an AAV p40 promoter, a p5 promoter, and/or a p19 promoter.

Optionally, the at least one cap gene promoter comprises an AAV p40 promoter, a p5 promoter, and a p19 promoter. However, any suitable promoter that is able to drive cap gene expression can be used.

Whether or not a given cap gene promoter is able to drive cap gene expression may be determined using an AAV production assay as described above. Specifically, if the cap gene promoter can drive cap gene expression it will facilitate AAV production. In this case, the test two-plasmid system will comprise a vector plasmid that comprises the cap gene and a cap gene promoter whose ability to facilitate AAV production is to be tested, and otherwise the test two-plasmid system used will be identical to the reference two-plasmid system. In one embodiment, the cap gene promoter will be considered to facilitate AAV production if it supports rAAV production at a level at least 25%, at least 40%, at least 50%, at least 60%, at least 70%, at least 80%, at least 90% or at least 95% of the level supported by a wild type p40 cap gene promoter, i.e. if the yield of rAAV vectors produced is at least 25%, at least 40%, at least 50%, at least 60%, at least 70%, at least 80%, at least 90% or at least 95% of the yield of rAAV produced using the reference two-plasmid system. Preferably, the cap gene promoter will be considered to facilitate AAV production if it supports rAAV production at a level at least 70%, at least 80%, at least 90% or at least 95% of the yield of rAAV produced using the reference two-plasmid system.

The p40 promoter is understood to drive expression of the cap gene. The native p40 promoter is contained within the native rep gene. The AAV2 p40 promoter has a sequence of nucleotides 1710-1827 of SEQ ID NO: 1. However, p40 promoters in other serotypes of AAV may comprise slightly different sequences. In some embodiments, the p40 promoter has a sequence at least 95%, at least 98%, or 99% identical to nucleotides 1710-1827 of SEQ ID NO: 1, or a corresponding sequence from another serotype of AAV. In some embodiments, the p40 promoter has a sequence at least 98% identical to nucleotides 1710-1827 of SEQ ID NO: 1.

The present inventors have found that an at least one cap gene promoter that comprises a p40 promoter, a p5 promoter and a p19 promoter is advantageous, as the p5 promoter and the p19 promoter play a role in regulating the p40 promoter, and the presence of the p40 promoter, the p5 promoter and the p19 promoter leads to timely regulated expression of the cap gene resulting in high yield/high quality rAAV. Accordingly, in some embodiments, the at least one cap gene promoter is comprised in a ‘promoter region’ comprising a p40 promoter, a p5 promoter and a p19 promoter. Optionally, the promoter region comprises a sequence at least 95%, at least 98%, at least 99% or 100% identity to the full length or a fragment of at least 800, at least 900, at least 1000 or at least 1100 nucleotides in length of the following stretches of native AAV2 sequence (SEQ ID NO: 1) positioned in immediate juxtaposition from 5′ to 3′: 200-354; 600-1049; 1701-2202, or to corresponding juxtaposed stretches of nucleotides from a different serotype of AAV.

The native p5 promoter is upstream of the native rep gene. The AAV2 p5 promoter has a sequence of nucleotides 204-292 of SEQ ID NO: 1. However, p5 promoters in other serotypes of AAV may comprise slightly different sequences. In some embodiments, the p5 promoter has a sequence at least 95%, at least 98%, or at least 99% identical to nucleotides 204-292 of SEQ ID NO: 1, or a corresponding sequence from another serotype of AAV. In some embodiments, the p5 promoter has a sequence at least 98% identical to nucleotides 204-292 of SEQ ID NO: 1.

The p19 promoter is contained within the native rep gene. The AAV2 p19 promoter has a sequence of nucleotides 730-890 of SEQ ID NO: 1. However, p19 promoters in other serotypes of AAV may comprise slightly different sequences. In some embodiments, the p19 promoter has a sequence at least 95%, at least 98%, or at least 99% identical to nucleotides 730-890 of SEQ ID NO: 1, or a corresponding sequence from another serotype of AAV. In some embodiments, the p19 promoter has a sequence at least 98% identical to nucleotides 730-890 of SEQ ID NO: 1.

An Expression Cassette Flanked on at Least One Side by an ITR

The vector plasmid or two-plasmid system of the invention may be used to produce AAV vector for use in gene therapy. A “gene therapy” involves administering AAV/viral particles of the invention that is capable of expressing a transgene (such as a Factor IX-encoding nucleotide sequence) in the host to which it is administered. In such cases, the vector plasmid will comprise an expression cassette.

Optionally, the vector plasmid comprises at least one ITR. Thus, optionally, the vector plasmid comprises at least one ITR, but, more typically, two ITRs (generally with one either end of the expression cassette, i.e. one at the 5′ end and one at the 3′ end). There may be intervening sequences between the expression cassette and one or more of the TTRs. The expression cassette may be incorporated into a viral particle located between two regular ITRs or located on either side of an ITR engineered with two D regions. Optionally, the vector plasmid comprises ITR sequences which are derived from AAV1, AAV2, AAV4 and/or AAV6. Preferably the ITR sequences are AAV2 ITR sequences.

Optionally, the vector plasmid comprises an expression cassette. As described herein, an expression cassette refers to a sequence of nucleic acids comprising a transgene and a promoter operably linked to the transgene. Optionally, the cassette further comprises additional transcription regulatory elements, such as enhancers, introns, untranslated regions, transcriptional terminators, etc.

Optionally, the expression cassette comprises a transcription regulatory element comprising the promoter element and/or enhancer element from HLP2, HLP1, LP1, HCR-hAAT, ApoE-hAAT, and/or LSP. These transcription regulatory elements are described in more detail in the following references: HLP2: WO16/075473; HLP1: McIntosh J. et al., Blood 2013 Apr. 25, 121(17):3335-44; LP1: Nathwani et al., Blood. 2006 Apr. 1, 107(7): 2653-2661; HCR-hAAT: Miao et al., Mol Ther. 2000; 1: 522-532; ApoE-hAAT: Okuyama et al., Human Gene Therapy, 7, 637-645 (1996); and LSP: Wang et al., Proc Natl Acad Sci USA. 1999 Mar. 30, 96(7): 3906-3910. Each of these transcription regulatory elements comprises a promoter, an enhancer, and optionally other nucleotides. If the polynucleotide is intended for expression in the liver, the promoter may be a liver-specific promoter. Optionally, the promoter is a human liver-specific promoter.

The transgene may be any suitable gene. If the vector plasmid is for use in gene therapy, the transgene may be any gene that comprises or encodes a protein or nucleotide sequence that can be used to treat a disease. For example, the transgene may encode an enzyme, a metabolic protein, a signalling protein, an antibody, an antibody fragment, an antibody-like protein, an antigen, or a non-translated RNA such as an miRNA, siRNA, snRNA, or antisense RNA.

Optionally, the transgene encodes a protein selected from the group consisting of Factor IX, α-Galactosidase A, beta-Glucocerebrosidase and Factor VIII.

Factor IX is a serine protease, which forms part of the coagulation cascade. The Factor IX protein may be a wild type Factor IX protein. The Factor IX protein may be a fragment of a wild type Factor IX protein. The Factor IX protein may be a variant of a wild type Factor IX protein. The Factor IX protein or fragment thereof may comprise one or more substitutions, deletions and/or additions in comparison to a wild type Factor IX protein. The Factor IX protein may be at least 90% identical to a wild type Factor IX protein. Preferably the Factor IX protein or fragment thereof is functional. Optionally the Factor IX protein or fragment thereof is hyper functional. A functional Factor IX protein or fragment thereof is one which carries out hydrolysis of an arginine-isoleucine bond in Factor X to form Factor Xa.

The Factor VIII protein may be a wild type Factor VIII protein. The Factor VIII protein may be a fragment of a wild type Factor VIII protein. The Factor VIII protein may be a variant of a wild type Factor VIII protein. The Factor VIII protein or fragment thereof may comprise one or more substitutions, deletions and/or additions in comparison to a wild type Factor VIII protein. The Factor VIII protein may comprise a deletion, such as a beta domain deletion. The Factor VIII protein may be at least 90% identical to a wild type Factor VIII protein. The Factor VIII protein may be at least 90% identical to a wild type Factor VIII protein comprising a deletion, such as a beta domain deletion.

Preferably the Factor VIII protein or fragment thereof is functional. Optionally the Factor VIII protein or fragment thereof is hyper functional. A functional Factor VIII protein or fragment thereof is one which can, when activated by thrombin, form an enzymatic complex with Factor IXa, phospholipids and calcium, and the enzymatic complex can catalyse the conversion of Factor X to Factor Xa.

The alpha-Galactosidase A protein may be a wild type alpha-Galactosidase A protein. The alpha-Galactosidase A protein may be a fragment of a wild type alpha-Galactosidase A protein. The alpha-Galactosidase A protein may be a variant of a wild type alpha-Galactosidase A protein. The alpha-Galactosidase A protein or fragment thereof may comprise one or more substitutions, deletions and/or additions in comparison to a wild type alpha-Galactosidase A protein. The alpha-Galactosidase A protein may be at least 90% identical to a wild type alpha-Galactosidase A protein. Preferably the alpha-Galactosidase A protein or fragment thereof is functional. Optionally the alpha-Galactosidase A protein or fragment thereof is hyper functional. A functional alpha-Galactosidase A protein or fragment thereof is one which hydrolyses the terminal alpha-galactosyl moieties from glycolipids and glycoproteins.

The beta-Glucocerebrosidase protein may be a wild type beta-Glucocerebrosidase protein. The beta-Glucocerebrosidase protein may be a fragment of a wild type beta-Glucocerebrosidase protein. The beta-Glucocerebrosidase protein may be a variant of a wild type beta-Glucocerebrosidase protein. The beta-Glucocerebrosidase protein or fragment thereof may comprise one or more substitutions, deletions and/or additions in comparison to a wild type beta-Glucocerebrosidase protein. The beta-Glucocerebrosidase protein may be at least 90% identical to a wild type beta-Glucocerebrosidase protein. Preferably the beta-Glucocerebrosidase protein or fragment thereof is functional. Optionally the beta-Glucocerebrosidase protein or fragment thereof is hyper functional.

A functional beta-Glucocerebrosidase protein or fragment thereof is one which hydrolyses the terminal alpha-galactosyl moieties from glycolipids and glycoproteins. A functional beta-Glucocerebrosidase protein or fragment is one which carries out hydrolysis of glucocerebroside.

Optionally, the vector plasmid comprises a cap gene and further comprises an expression cassette flanked on at least one side by an ITR.

Dispensable Translation Initiation Codons

Optionally, the vector plasmid does not comprise any dispensable translation initiation codons. Optionally, the vector plasmid may comprise at least two genes that must be capable of being transcribed and translated (the transgene (in an expression cassette) and the cap gene). Optionally, the transgene encodes a functional RNA which does not encode a translational protein or polypeptide. The transgene, if protein-encoding, and the cap gene will comprise start (ATG or GTG) codons to promote initiation of translation of the gene. However, the vector plasmid may comprise additional instances of ATG or GTG (either in-frame or out of frame with the reading frame of these genes), and it is possible that translation may initiate at one of these positions. Since there is no need for translation to be initiated at the site of these additional instances of ATG or GTG, the ATG or GTG codons may be considered to be “dispensable translation initiation codons”. It is preferred that dispensable translation initiation codons are removed or rendered non-functional, in particular where they occur in the promoter region. The promoter region is a region of the vector plasmid that comprises one or more promoters operably linked to the cap gene. In some embodiments, the cap gene is operably linked to one or more of the p5, p19 and p40 promoters, and in such embodiments the promoter region comprises the p5, p19 and p40 promoters.

Optionally, the plasmid comprises a promoter region comprising one or more promoters, and the promoter region does not comprise ATG or GTG codons. Optionally, the promoter region comprises p5, p19 and/or p40 promoters, and wherein ATG or GTG codons at one or more positions corresponding to positions (a) 321-323 (Rep78/68 ATG start codon), (b) 766-768 (ATG codon), (c) 955-957 (ATG codon), (d) 993-995 (Rep52/40 ATG start codon) and (e) 1014-1016 (GTG codon) of SEQ ID NO: 1 are absent or mutated.

Optionally, in the promoter region:
(a) nucleotides corresponding to nucleotides 321-323 of SEQ ID NO: 1 are absent;
(b) nucleotides corresponding to nucleotides 766-768 of SEQ ID NO: 1 are not ATG and are optionally ATT;
(c) nucleotides corresponding to nucleotides 955-957 of SEQ ID NO: 1 are absent;
(d) nucleotides corresponding to nucleotides 993-995 of SEQ ID NO: 1 are absent; and/or
(e) nucleotides corresponding to nucleotides 1014-1016 of SEQ ID NO: 1 are absent.

Optionally, in the promoter region:
(a) nucleotides corresponding to nucleotides 321-323 of SEQ ID NO: 1 are absent;
(b) nucleotides corresponding to nucleotides 766-768 of SEQ ID NO: 1 are not ATG and are optionally ATT;
(c) nucleotides corresponding to nucleotides 955-957 of SEQ ID NO: 1 are absent;
(d) nucleotides corresponding to nucleotides 993-995 of SEQ ID NO: 1 are absent; and
(e) nucleotides corresponding to nucleotides 1014-1016 of SEQ ID NO: 1 are absent.

Size of the Vector Plasmid

As discussed above in connection with the helper plasmid, it is advantageous that plasmids used in the production of rAAV are as small as possible. Smaller plasmids are more cost-effective to produce and are easier to transfect into cells.

The size of the vector plasmid will in part be defined by the size of the promoters, transcription regulatory elements, transgenes and cap genes that are included, and as discussed above, the nature of these features will be partly or fully defined by the application for which the rAAV is to be used.

However, the size of the vector plasmid can be reduced by ensuring that the size of the backbone that is used is small, and/or by ensuring that the vector plasmid does not comprise a spacer (a contiguous regions of non-coding nucleic acid sequences of greater than 200 nucleotides in length).

Optionally, the vector plasmid comprises a backbone of fewer than 4000 nucleotides, fewer than 3500 nucleotides, fewer than 3000 nucleotides, or fewer than 2500 nucleotides in length. Optionally, the vector plasmid does not comprise any spacers.

Host Cell

The present invention provides a host cell comprising the two-plasmid system, the helper plasmid or the vector plasmid of the invention.

The host cell is preferably a host cell that can be used to produce rAAV. The host cell may therefore be a host cell suitable for the production of rAAV. In addition, the host cell may be for the production of rAAV.

In general a host cell that is suitable for the production of rAAV is a host cell that is derived from a eukaryotic cell line, preferably a vertebrate cell line, preferably a mammalian cell line, preferably a human cell line. Optionally, the host cell is a cell selected from the group consisting of a HEK293T cell, a HEK293 cell, a HEK293EBNA cell, a CAP cell, a CAP-T cell, an AGE1.CR cell, a PerC6 cell, a C139 cell, an EB66 cell, a BHK cell, a COS cell, a Vero cell, a Hela cell, and an A549 cell. Preferably, the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell. Even more preferably, the host cell is selected from the group consisting of a HEK293T cell, a HEK293 cell, and a HEK293EBNA cell. For example, the host cell may be a HEK293T cell. Optionally, the host cell is a cell that expresses a functional adenoviral E1A/B protein. For example, the host cell may comprise a chromosome comprising a gene encoding a functional adenoviral E1A/B protein. Functional adenoviral E1A/B proteins are discussed in the section entitled “At least one helper virus gene”.

It is within the abilities of the skilled person to determine whether a host cell is suitable for the production of rAAV. The skilled person merely needs to determine whether the host cell supports AAV production using an AAV production assay as described above.

In this case, the test two-plasmid system and the reference two-plasmid system that are used will be identical. However, the test two-plasmid system will be transfected into the host cell whose suitability for the production of recombinant AAV is to be tested, and the reference two-plasmid system will be transfected into HEK293T cells. The host cell will be considered to be suitable for the production of recombinant AAV if it supports AAV production at a level at least 30%, at least 40%, at least 50%, at least 70%, at least 80%, at least 90% or at least 95% of the level supported by HEK293T cells, i.e. if the yield of recombinant AAV produced is at least 30%, at least 40%, at least 50%, at least 70%, at least 80%, at least 90% or at least 95% of the yield of recombinant AAV produced in HEK293T cells.

A Low Level of Replication Competent AAV (rcAAV)

The present invention provides a use of the two-plasmid system, the helper plasmid or the vector plasmid of the invention for producing a rAAV preparation having a low level of replication competent AAV (rcAAV).

The present invention also provides a use of the two-plasmid system, the helper plasmid or the vector plasmid of the invention for reducing or minimising the level of rcAAV produced during rAAV production.

The present invention also provides a use of the two-plasmid system, the helper plasmid or the vector plasmid of the invention for reducing or minimising the level of pseudo-wild type rcAAV produced during rAAV production.

The present invention also provides a method for producing a rAAV preparation comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production
wherein the rAAV preparation comprises a low level of replication competent AAV (rcAAV).

The method may further comprise a step of harvesting the rAAV to provide an rAAV preparation comprising a low level of rcAAV.

The present invention also encompasses a method for reducing or minimising the level of replication competent AAV (rcAAV) produced during recombinant AAV (rAAV) production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

The method may further comprise a step of harvesting the rAAV to provide an rAAV preparation, optionally having a low level of rcAAV.

The present invention also encompasses a method for reducing or minimising the level of pseudo-wild type replication competent AAV (rcAAV) produced during recombinant AAV (rAAV) production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

The method may further comprise a step of harvesting the rAAV to provide an rAAV preparation, optionally having a low level of pseudo-wild type rcAAV.

“RAAV” or “recombinant AAV” refers to AAV particles, i.e. particles comprising an AAV genome (such as a vector genome) and an AAV capsid.

For the purposes of the present invention, the terms “rcAAV” or “replication competent AA V” is intended to refer to rAAV particles that comprise a genome which comprises a rep gene or a genome which comprises a cap gene. Whilst, for the purposes of the present invention, the term “rcAAV” is intended to refer to rAAV particles that comprise a genome comprising a rep gene or a cap gene, it is understood that the rAAV particles that comprise a genome comprising a rep gene but not a cap gene (herein so-called “cap-deficient rcAAV”) or a genome comprising a cap gene but not a rep gene (herein so-called “rep-deficient rcAAV”) are not replication competent in isolation. Rather, in order to replicate (in the presence of the necessary helper virus functions, but without any requirement for AAV functions provided in trans) an AAV particle must comprise a rep gene and a cap gene (so-called “pseudo-wild type rcAAV”). However, an AAV particle that comprises a rep gene but does not comprise a cap gene can replicate when co-infected in a host cell with an AAV particle that comprises a cap gene, and an AAV particle that comprises a cap gene but does not comprise a rep gene can replicate when co-infected in a host cell with an AAV particle that comprises a rep gene. Thus, the term “rcAAV” encompasses “cap-deficient rcAAV”, but also “pseudo-wild type rcAAV” and “rep-deficient rcAAV”. Pseudo-wild type rcAAV may (in the presence of helper virus functions) be able to replicate in a host cell without co-infection by another AAV particle, and produce progeny virus. Pseudo-wild type rcAAV may comprise a rep gene and a cap gene flanked by ITR sequences.

The term “rep-rcAAV” encompasses “pseudo-wild type rcAAV” and “cap-deficient rcAAV”. The term “cap-rcAAV” encompasses “pseudo-wild type rcAAV” and “rep-deficient rcAAV”.

All species of rcAAV are undesirable in an rAAV preparation. Cap-deficient rcAAV may replicate when co-infected with cap-containing AAV particles. Rep-deficient rcAAV may replicate when co-infected with rep-containing AAV particles, and pseudo-wild type rcAAV may replicate without AAV co-infection. If the rcAAV replicates, it can cause significant side effects. Accordingly, reducing or minimising the level of rcAAV produced is advantageous. As used herein, the term “reducing the level of rcAAV” refers to decreasing the number of rcAAV that are produced. As used herein the term “minimising the level of rcAAV” refers to reducing the level of rcAAV to the lowest level possible given the constraints of the method.

Using the methods of the invention, the rAAV genome that is packaged should not comprise either a rep gene or a cap gene as, in general, neither the rep gene nor the cap gene is linked (on the same plasmid, in close proximity) to an ITR. However, single recombination events could result in the generation of cap-deficient or rep-deficient rcAAV. To generate pseudo-wild-type rcAAV, two recombination events must occur, and this is highly unlikely.

Whether or not an rAAV particle comprises a genome comprising a rep gene, or a preparation of rAAV particles comprises a particle comprising a genome comprising a rep gene, may be determined using qPCR. Optionally, the amount of rcAAV is measured by determining the copy number of rep produced. Optionally, the amount of rcAAV is measured by determining the copy number of rep per cell (i.e. host cell) using qPCR.

A primer (or two primers) specific for the rep gene should be used in the qPCR. Optionally, the (or both) primer(s) is/are specific for (reverse and complementary to or identical to depending on whether the primer is a forward primer or a reverse primer) a region of at least 12, at least 14, at least 16, or at least 18 of nucleotides 321-2252 of SEQ ID NO: 1, which encode the four Rep proteins. Optionally, the primer is specific for rep 40. Optionally, the primer is specific for rep 52. Optionally, the primer is specific for rep 68.

To determine the copy number of rep per cell using qPCR a second pair of primers should be used in the qPCR, which is specific for an endogenous gene (such as an endogenous housekeeping gene) in the host cell such as human albumin in HEK293 cells. Optionally, each primer or pair of primers is specific for (complementary to) a region of at least 8, at least 10, at least 12 or at least 15 nucleotides of the genomic sequence of human albumin. The copy number per cell may then be determined as the ratio of the copy number of rep to the copy number of the endogenous gene.

Alternatively, the above method to detect/measure rcAAV (pseudo-wild type and/or cap-deficient rcAAV) may be performed by cap gene-specific qPCR which can be used to detect rep-deficient rcAAV.

The methods or uses may provide an rAAV preparation. In such embodiments, the rAAV preparation preferably comprises a low level of rcAAV. A low level of rcAAV is generally a level of rcAAV that is lower than 1 rcAAV in 10⁷ rAAV, or is lower than that in an rAAV preparation that is produced using a method equivalent to that of a method of the invention, except that the vector plasmid comprises both at least one rep gene and at least one cap gene. The level of rAAV can be determined by using qPCR to determine the number of vector genomes, as described below in the section entitled “high yield”.

Optionally, the level of rcAAV is less than 1 rcAAV in 10⁷ recombinant AAV, less than 1 rcAAV in 10⁹ recombinant AAV, or less than 1 rcAAV in 10¹⁰ recombinant AAV. Optionally, the level of rcAAV is less than the level of rcAAV produced using an equivalent method except that the vector plasmid comprises both the at least one rep gene and at least one cap gene. Optionally, the level of rcAAV is measured using the qPCR assay above. Optionally, the level of rcAAV is measured using the qPCR assay after two or three generations of culturing. Optionally, the rcAAV is pseudo-wild type rcAAV. Optionally, the rcAAV is cap-deficient rcAAV. Optionally, the rcAAV is rep-deficient rcAAV. Optionally, the rcAAV comprises pseudo-wild type rcAAV, cap-deficient rcAAV and rep-deficient rcAAV.

The phrase “method equivalent to that of a method of the invention” is intended to refer to a method that is identical, except that the vector plasmid comprises both the at least one rep gene and at least one cap gene.

Optionally, the low level of rcAAV comprises a low level of rep-rcAAV. When the rcAAV “comprises a low level of rep-rcAAV”, then the rcAAV present in the preparation has at most a small proportion of rep-rcAAV. Thus, the phrase “low level of rcAAV comprises a low level of rep-rcAAV” as used herein means that the low level of rcAAV present in a preparation has at most a small proportion of rep-rcAAV. Optionally, the low level of rcAAV comprises a lower level of rep-rcAAV compared to the level of rep-rcAAV produced using a two-plasmid system comprising a plasmid comprising both at least one rep gene and at least one cap gene.

The two-plasmid system comprising a plasmid comprising both at least one rep gene and at least one cap gene is also referred to herein as “the two-plasmid system used for comparison”. The two-plasmid system used for comparison may be in a conventional “non-split” configuration. The plasmid comprising both at least one rep gene and at least one cap gene (of the two-plasmid system used for comparison) may comprise all four rep genes (e.g. the AAV2 rep cassette). The plasmid comprising both at least one rep gene and at least one cap gene (of the two-plasmid system used for comparison) may comprise the same cap gene sequence as the two-plasmid system of the invention. The plasmid comprising both at least one rep gene and at least one cap gene (of the two-plasmid system used for comparison) may comprise the same rep gene sequence as the two-plasmid system of the invention. The two-plasmid system used for comparison may comprise a plasmid comprising the same AdV (adenovirus) helper and vector genome sequences (e.g. expression cassette) as the two-plasmid system of the invention. For example, the two-plasmid system used for comparison may comprise a plasmid which comprises the same AdV helper and vector genome sequences (e.g. expression cassette) as the two-plasmid system of the invention, and the plasmid comprising both at least one rep gene and at least one cap gene (of the two-plasmid system used for comparison) comprises the same cap gene sequence and the same rep gene sequence as the two-plasmid system of the invention. As a further example, the two-plasmid system used for comparison may comprise a plasmid which comprises the same AdV helper and vector genome sequences (e.g. expression cassette) as the two-plasmid system of the invention, and the plasmid comprising both at least one rep gene and at least one cap gene (of the two-plasmid system used for comparison) comprises the same cap gene sequence as two-plasmid system of the invention and a AAV (e.g. AAV2) rep cassette comprising all four rep genes. Optionally, the plasmids of the two-plasmid system used for comparison are transfected in a molar plasmid ratio of 1.8:1 (AdV helper-vector genome plasmid: rep-cap plasmid).

Optionally, the low level of rcAAV comprises an at least 5 times, at least 10 times, at least 25 times, at least 30 times, or at least 35 times excess of rep-rcAAV or cap-rcAAV. Optionally, the low level of rcAAV comprises an at least 5 times, at least 10 times, at least 25 times, at least 30 times, or at least 35 times excess of cap-rcAAV, i.e. the rcAAV may comprise at least 5 times, at least 10 times, at least 25 times, at least 30 times, or at least 35 times more cap sequences than rep sequences. Optionally, the low level of rcAAV comprises between an at least 5 times and an at least 100 times, or between an at least 25 times and an at least 50 times excess of cap-rcAAV, i.e. the low level of rcAAV may comprise between at least 5 times and at least 100 times, or between at least 25 times and at least 50 times more cap sequences than rep sequences. Where the level of cap-rcAAV is in excess, then the level of rep-rcAAV must be lower. A lower level of rep-rcAAV indicates a low level of pseudo wild-type rcAAV (because pseudo-wild type rcAAV comprises the rep gene).

Optionally, the low level of rcAAV comprises an at least 5 times, at least 10 times, at least 25 times, at least 30 times, or at least 35 times excess of rep-rcAAV, i.e. the rcAAV may comprise at least 5 times, at least 10 times, at least 25 times, at least 30 times, or at least 35 times more rep sequences than cap sequences. Optionally, the low level of rcAAV comprises between an at least 5 times and an at least 100 times, or between an at least 25 times and an at least 50 times excess of rep-rcAAV, i.e. the low level of rcAAV may comprise between at least 5 times and at least 100 times, or between at least 25 times and at least 50 times more rep sequences than cap sequences. Where the level of rep-rcAAV is in excess, then the level of cap-rcAAV must be lower. A lower level of cap-rcAAV indicates a low level of pseudo wild-type rcAAV (because pseudo-wild type rcAAV comprises the cap gene).

The likelihood of whether a rep or cap gene is packaged into an AAV particle is increased if the plasmid comprising the rep or cap gene also comprises at least one ITR sequence.

For example, if one plasmid comprises the rep gene and does not comprise at least one ITR sequence, and another plasmid comprises the cap gene and at least one ITR sequence, then there is likely to be more cap gene packaged than rep gene. As a further example, if one plasmid comprises the cap gene and does not comprise at least one ITR sequence, and another plasmid comprises the rep gene and at least one ITR sequence, then there is likely to be more rep gene packaged than cap gene.

Optionally, the low level of rcAAV comprises a proportion of less than ⅕ rep-rcAAV, less than 1/10 rep-rcAAV, less than 1/25 rep-rcAAV, less than 1/30 rep-rcAAV, or less than 1/35 rep-rcAAV. As an example, where the rcAAV comprises a proportion less than ⅕ rep-rcAAV, then at least ⅘ of the rcAAV must be cap-rcAAV.

Optionally, the low level of rcAAV comprises a proportion of rep-rcAAV that is less than the proportion produced using a two-plasmid system comprising a plasmid comprising both at least one rep gene and at least one cap gene. Optionally, the proportion of rep-rcAAV is less than 90%, less than 75%, less than 60%, less than 50%, less than 25%, less than 20%, less than 17%, or less than 15% of the proportion produced using the two-plasmid system comprising a plasmid comprising both at least one rep gene and at least one cap gene. The two-plasmid system comprising a plasmid comprising both at least one rep gene and at least one cap gene is also referred to herein as the two-plasmid system used for comparison.

Optionally, the low level of rcAAV comprises a low level of pseudo-wild type rcAAV.

Optionally, the low level of rcAAV comprises less than ⅕ pseudo-wild type rcAAV, less than 1/10 pseudo-wild type rcAAV, less than 1/25 pseudo-wild type rcAAV, less than 1/30 pseudo-wild type rcAAV, or less than 1/35 pseudo-wild type rcAAV.

In the rcAAV produced, where the quantities of rep and cap sequences are not equal, the lower amount between the quantities of rep and cap sequences is an indicator of the maximum possible number of AAV species which contain rep and cap genes on the same DNA molecule. Therefore, the lower amount between the quantities of rep and cap sequences is an indicator of the maximum possible number of pseudo-wild type rcAAV.

Thus, where either the rep-rcAAV or cap-rcAAV is in excess, it is likely that the amount of pseudo-type rcAAV is lower than if the levels of rep-rcAAV and cap-rcAAV were equal. The lower amount between the quantities of rep and cap sequences as a proportion of the upper amount between the quantities of rep and cap sequences is an indicator of the proportion of rcAAV which is pseudo-wild type rcAAV. As an example, if there are 40 times more cap sequences than rep sequences, then the proportion of rcAAV which is pseudo-wild type rcAAV may be 1/40. In the rcAAV produced, the proportion of rcAAV which is pseudo-wild type rcAAV may be less than ⅕, less than 1/10, less than 1/25, less than 1/30, or less than 1/35.

The quantity of cap and rep sequences may be measured by qPCR. Suitable qPCR is discussed in Example 10. Optionally, the level of rep-rcAAV is the level of rep-rcAAV detected by qPCR using primers binding to rep68 exon 1. Optionally, the level of rep-rcAAV is the level of rep-rcAAV detected by qPCR using the forward primer CACGTGCATGTGGAAGTAG (SEQ ID NO: 7) and the reverse primer CGACTTTCTGACGGAATGG (SEQ ID NO: 8). Optionally, the level of cap-rcAAV is the level of cap-rcAAV detected by qPCR using primers binding to a sequence encoding VP3. Optionally, the level of cap-rcAAV is the level of cap-rcAAV detected by qPCR using the forward primer TACTGAGGGACCATGAAGAC (SEQ ID NO: 9) and the reverse primer GTTTACGGACTCGGAGTATC (SEQ ID NO: 10).

Optionally, an excess of rep-rcAAV or cap-rcAAV indicates that the level of pseudo wild-type rcAAV is reduced or minimised. Optionally, the excess is at least 5 times, at least 10 times, at least 25 times, at least 30 times, or at least 35 times excess of rep-rcAAV or cap-rcAAV. Optionally, the excess is between at least 5 times and at least 100 times, or between at least 25 times and at least 50 times excess of rep-rcAAV or cap-rcAAV. Optionally, the low level of rcAAV comprises an at least 5 times, at least 10 times, at least 25 times, at least 30 times, or at least 35 times excess of cap-rcAAV, i.e. the rcAAV may comprise at least 5 times, at least 10 times, at least 25 times, at least 30 times, or at least 35 times more cap sequences than rep sequences. Optionally, the low level of rcAAV comprises between an at least 5 times and an at least 100 times, or between an at least 25 times and an at least 50 times excess of cap-rcAAV, i.e. the low level of rcAAV may comprise between at least 5 times and at least 100 times, or between at least 25 times and at least 50 times more cap sequences than rep sequences. Where the level of cap-rcAAV is in excess, then the level of rep-rcAAV must be lower. A lower level of rep-rcAAV indicates a low level of pseudo wild-type rcAAV (because pseudo-wild type rcAAV comprises the rep gene). Optionally, the low level of rcAAV comprises an at least 5 times, at least 10 times, at least 25 times, at least 30 times, or at least 35 times excess of rep-rcAAV, i.e. the rcAAV may comprise at least 5 times, at least 10 times, at least 25 times, at least 30 times, or at least 35 times more rep sequences than cap sequences. Optionally, the low level of rcAAV comprises between an at least 5 times and an at least 100 times, or between an at least 25 times and an at least 50 times excess of rep-rcAAV, i.e. the low level of rcAAV may comprise between at least 5 times and at least 100 times, or between at least 25 times and at least 50 times more rep sequences than cap sequences. Where the level of rep-rcAAV is in excess, then the level of cap-rcAAV must be lower. A lower level of cap-rcAAV indicates a low level of pseudo wild-type rcAAV (because pseudo-wild type rcAAV comprises the cap gene). Optionally, the excess is greater than the excess produced using a two-plasmid system comprising a plasmid comprising both at least one rep gene and at least one cap gene. The two-plasmid system comprising a plasmid comprising both at least one rep gene and at least one cap gene is also referred to herein as the two-plasmid system used for comparison.

Optionally, the quantity of cap and rep sequences an rAAV preparation is measured following at least one round of amplification. For example, the quantity of cap and rep sequences in an rAAV preparation may be measured following two rounds of amplification. To perform a round of amplification, optionally, suitable host cells (such as HEK293T cells) are infected with the rAAV preparation, co-infected with adenovirus and incubated under conditions suitable for the production of rAAV. The cells may be lysed and the quantity of cap and rep sequences in the lysate measured. Optionally, the lysate may be used for a second round of amplification. For example, the lysate may be used to infect further suitable host cells (such as HEK293T cells) which are also co-infected with adenovirus and incubated under conditions suitable for the production of rAAV. The cells may be lysed and the quantity of cap and rep sequences in the lysate measured. Suitable methods for amplification are set out in Example 8.

A Desired Ratio of Full to Total Particles

The present invention provides a use of the two-plasmid system, the helper plasmid or the vector plasmid of the invention for producing an rAAV preparation having a desired ratio of full to total particles.

The present invention also provides a use of the two-plasmid system, the helper plasmid or the vector plasmid of the invention for controlling or maximising the ratio of full to total particles produced during rAAV production.

The present invention also provides a method for producing an rAAV preparation comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production
wherein the rAAV preparation comprises a desired ratio of full to total particles.

The present invention also encompasses a method for controlling or maximising the ratio of full to total particles produced during rAAV production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

The method may further comprise a step of harvesting the rAAV to provide an rAAV preparation, optionally having a desired ratio of full to total particles.

The present inventors have determined that altering the ratio of helper plasmid to vector plasmid can be used to alter the ratio of full to total particles that are produced.

Specifically, the inventors have determined that increasing the level of the vector plasmid relative to the helper plasmid decreases the ratio of full to total particles.

“Full” particles are rAAV particles comprising both a capsid and the intended vector genome, or at least a partial such genome as determined using the qPCR method described below. “Empty” particles (i.e. particles which are not full) comprise capsids but do not comprise a genome, or comprise only a partial genome (thereby not forming a complete rAAV particle) which is not detected using the qPCR method. However, the rAAV preparations may comprise both full particles and empty particles. Generally a low or minimised proportion of empty particles is desired. For example, if the rAAV are to be used in gene therapy, any empty particles will not comprise the entire expression cassette of interest and so will not be effective in therapy. On the other hand, there are circumstances where the presence of empty particles could be desirable. In some instances, and in some patient groups, it may be the case the empty particles behave as “decoys” to reduce the immune response in a patient to the administered rAAV particles (WO2013/078400). Furthermore, as will be discussed in more detail below, whilst increasing the level of the vector plasmid relative to the helper plasmid decreases the ratio of full to total particles, it also increases the yield of rAAV produced (and thereby in some cases the total number of full particles that are produced). Thus, even if the user of the method believes that a high full to total particle ratio is desirable, the user may use a lower proportion of helper plasmid if this leads to a greater yield. Alternatively, if the user, for example, implements a process change during development of a given product, e.g. switches from bioreactor A to bioreactor B resulting in different full to total particle ratios when keeping the plasmid ratio constant, the plasmid ratio can be altered to compensate for or offset the effect of the process change to ensure consistent full to total particle ratio throughout development, as this ratio is a critical quality parameter of a rAAV batch that needs to be controlled and kept comparable from batch to batch.

Accordingly, an advantage of the methods and uses of the present invention is that they allow the user the flexibility to determine a desirable ratio of full to total particles and modify the ratio of helper plasmid to vector plasmid in order to achieve that desired ratio of full to total particles.

The ratio of full to total particles may be expressed herein as the percentage of the total number of particles (capsids) that notionally comprise a vector genome or at least a partial such genome (assuming one (partial) genome per capsid) as determined using the following qPCR assay. qPCR is carried out using a pair of primers that are that are able to amplify at least a region of the promoter of the expression cassette. Optionally, at least one of the primers is specific for (reverse and complementary to or identical to depending whether the primer is a forward primer or a reverse primer) a region of at least 12, at least 14, at least 16, or at least 18 nucleotides of the promoter of the expression cassette.

Optionally, one primer is specific for the start of the promoter (the first at least 12 nucleotides of the promoter) and the other primer is specific for a region of the expression cassette that is 150 base pairs from the binding site of the first primer.

The ratio of full to total particles (as measured as a percentage of the total number of particles that are full particles) may be determined using qPCR to determine the number of vector genomes (as discussed in the previous paragraph), and using a capsid-specific ELISA to measure the total number of particles. For example, the capsid-specific ELISA may comprise exposing the rAAV preparation to an antibody that binds to the capsid protein. If, for example, the vector plasmid comprises a cap gene that encodes a capsid from an AAV2 serotype, the antibody may be an antibody that binds to the AAV2 capsid. For example, the user may coat a plate with an antibody that is specific for the capsid. The user may then pass the rAAV preparation over the surface of the plate. The particles will bind to the antibody and be immobilised on the plate. The plate may then be washed to remove contaminants. The amount of particle present can then be detected by addition of a detection antibody that can bind to the capsid and is conjugated to a detection agent such as streptavidin peroxidase. The amount of particle present will be proportional to the colour change obtained when the streptavidin peroxidase is exposed to the chromogenic substrate TMB (tetramethylbenzidine).

Optionally, the desired ratio of full to total particles (expressed as a percentage of the total number of particles that notionally comprise a vector genome) is at least 2%, at least 3%, at least 4%, at least 5%, at least 7%, at least 10% or at least 15% (as measured on bulk product prior to harvesting, purifying and/or concentrating the rAAV preparation). In many cases, increasing the full to total particle ratio is advantageous, and the present inventors have determined that aiming for a full to total particle ratio of at least 2%, at least 3%, at least 4%, at least 5%, at least 7%, at least 10% or at least 15% achieves a good balance between maintaining a high full to total particle ratio, whilst also achieving a good yield. Optionally, the desired ratio of full to total particles is a ratio of full to total particles that is at least 20% or at least 30% of the ratio of full to total particles achieved using an equivalent method with a ratio of helper plasmid to vector plasmid of 1.8:1. Optionally, the desired ratio of full to total particles is a ratio of full to total particles that is at least 40% or at least 45% of the ratio of full to total particles achieved using an equivalent method with a ratio of helper plasmid to vector plasmid of 1.8:1.

Optionally, the ratio of helper plasmid to vector plasmid that is used, selected or adjusted to is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; or between 1:1.5 and 1:3. Optionally, the ratio of helper plasmid to vector plasmid comprises a molar excess of vector plasmid. Optionally, the ratio of helper plasmid to vector plasmid that is used, selected or adjusted to is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; between 1:1.5 and 1:3; between 1:2 and 1:4; or around 1:3. Optionally, the ratio of helper plasmid to vector plasmid that is used, selected or adjusted to is between 1:2 and 1:4, or around 1:3. Preferably, the ratio of helper plasmid to vector plasmid that is used, selected or adjusted to is around 1:3.

High Yield

The present invention provides a use of the two-plasmid system, the helper plasmid or the vector plasmid of the invention for producing an rAAV preparation at a high or desired yield.

The present invention also provides a use of the two-plasmid system, the helper plasmid or the vector plasmid of the invention for increasing, optimising or maximising the yield of rAAV produced during rAAV production.

The present invention also provides a method for producing an rAAV preparation comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production
wherein the rAAV preparation is at a high or desired yield.

The present invention also encompasses a method for increasing, optimising or maximising the yield of rAAV produced during rAAV production comprising:
(a) obtaining the two-plasmid system, the helper plasmid or the vector plasmid of the invention;
(b) transfecting a host cell with the two-plasmid system, the helper plasmid or the vector plasmid of the invention; and
(c) culturing the host cell under conditions suitable for rAAV production.

The method may further comprise a step of harvesting the rAAV vector to provide a rAAV preparation, optionally at a high or desired yield. The term “yield” refers to the amount of AAV particles that are prepared in the methods or uses of the invention. The “yield” may be expressed as the number of vector genomes (vg) per ml of medium, as measured before (as measured on bulk product prior to harvesting, purifying and/or concentrating the rAAV preparation). The number of vector genomes can be measured as discussed under the heading “a desired ratio of full to total particles”, i.e. the yield of rAAV (such as rAAV particles) may be determined by using qPCR to quantify the number of nucleic acid sequences comprising a cassette comprising a promoter sequence (vg).

The present inventors have shown that modifying the ratio of helper plasmid to vector plasmid may be used to increase the yield (i.e. quantity of vgs or vg/ml produced) of a method that produces rAAV. Having the highest yield possible is clearly advantageous, as it reduces the amount of resources required to produce the rAAV. However, under some conditions increasing the yield to the highest possible level may result in a decrease in the ratio of full to total particles. Thus, if the user is producing the rAAV for an application where maximising the ratio of full to total particles is important, then he may opt to use a desired yield that is not the highest possible yield by using a helper plasmid to vector plasmid ratio that optimises in favour of the ratio of full to total (i.e. “% age full”) particles.

As used herein the term “maximising” the yield refers to aiming for the highest possible yield within the limitations of the methods of the invention. As used herein the term “optimising” refers to increasing the yield, but aiming for a desired yield which may not be the maximum possible yield if, for example, the user is keen to ensure a high ratio of full to total particles (i.e. minimising the proportion of empty particles).

As used herein the term “high yield” generally refers to a yield that is greater than 2×10¹⁰ vg/ml, or at least 2-fold greater than the yield achieved using an equivalent method with a ratio of helper plasmid to vector plasmid of 1.8:1. An “equivalent method” is a method that is identical, except that the ratio of helper plasmid to vector plasmid is 1.8:1.

Optionally, the high or desired yield is a yield greater than 2×10¹⁰ vg/ml, greater than 4×10¹⁰ vg/ml, greater than 6×10¹⁰ vg/ml, greater than 8×10¹⁰ vg/ml, greater than 1×10¹¹ vg/ml, greater than 2×10¹¹ vg/ml, or greater than 4×10¹¹ vg/ml.

Optionally, the high or desired yield is a yield that is at least 2-fold, at least 4-fold, at least 5-fold, or at least 6-fold higher than the yield achieved using an equivalent method with a ratio of helper plasmid to vector plasmid of 1.8:1.

Optionally, the ratio of helper plasmid to vector plasmid that is used, selected or adjusted to is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; or between 1:1.5 and 1:3. Optionally, the ratio of helper plasmid to vector plasmid comprises a molar excess of vector plasmid compared to helper plasmid. Optionally, the ratio of helper plasmid to vector plasmid that is used, selected or adjusted to is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; between 1:1.5 and 1:3; between 1:2 and 1:4; or around 1:3. Optionally, the ratio of helper plasmid to vector plasmid that is used, selected or adjusted to is between 1:2 and 1:4, or around 1:3. Preferably, the ratio of helper plasmid to vector plasmid that is used, selected or adjusted to is around 1:3.

Balance Between High Yield and High Full to Total Particle Ratio

As discussed above, the ratio of full to total particles and the yield may be affected by altering the ratio of helper plasmid to vector plasmid used in the methods. Thus, the user may adjust the ratio of helper plasmid to vector plasmid to a suitable ratio for obtaining the desired full to total particle ratio. Optionally, the methods or uses may comprise a step of selecting a ratio of helper plasmid to vector plasmid. Altering the ratio of helper plasmid to vector plasmid, or selecting a ratio of helper plasmid to vector plasmid, may be achieved by simply mixing the helper plasmid and the vector plasmid in different relative proportions.

The user may need to carry out a test method to determine the ratio of helper plasmid to vector plasmid that is required to obtain the desired ratio of full to total particles, but this is not burdensome. For example, the skilled person may perform a small scale experiment to determine the required ratio of helper plasmid to vector plasmid, and then this ratio can be used in a larger scale production process. Optionally, the step of selecting a ratio of helper plasmid to vector plasmid may comprise performing small scale test runs of the method of the invention using different ratios of helper plasmid to vector plasmid.

Optionally, the ratio of helper plasmid to vector plasmid is selected or adjusted to a ratio that achieves a balanced yield versus full to total particle ratio. Optionally, the ratio of helper plasmid to vector plasmid is selected or adjusted to a ratio that achieves a maximum yield of rAAV (i.e. vgs) with the minimum yield of particles achievable at such maximum yield of rAAV, i.e. the user maximises the yield, and then determines the helper plasmid to vector plasmid ratio that achieves that yield while providing the highest ratio of full to total particles.

Optionally, the ratio of helper plasmid to vector plasmid is adjusted to obtain the desired ratio of full to total particles and/or the high or desired yield of recombinant AAV. Optionally, the ratio of helper plasmid to vector plasmid is selected or adjusted to a ratio that allows the user to obtain the desired ratio of full to total particles or the high or desired yield of recombinant AAV.

Optionally, the ratio of helper plasmid to vector plasmid that is used, selected or adjusted to is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; or between 1:1.5 and 1:3. Optionally, the ratio of helper plasmid to vector plasmid comprises a molar excess of vector plasmid. Optionally, the ratio of helper plasmid to vector plasmid that is used, selected or adjusted to is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; between 1:1.5 and 1:3; between 1:2 and 1:4; or around 1:3. Optionally, the ratio of helper plasmid to vector plasmid that is used, selected or adjusted to is between 1:2 and 1:4, or around 1:3. Preferably, the ratio of helper plasmid to vector plasmid that is used, selected or adjusted to is around 1:3.

Transfecting, Culturing and Harvesting

The methods of the invention may comprise steps of obtaining the two-plasmid system, the helper plasmid or the vector plasmid of the invention, transfecting a host cell with the two-plasmid system, the helper plasmid or the vector plasmid, and culturing the host cell under conditions suitable for recombinant AAV production.

Transfecting a host cell with the two-plasmid system, the helper plasmid or the vector plasmid, may comprise exposing the host cell to the two-plasmid system, the helper plasmid or the vector plasmid in conditions suitable for transfection. For example, the user of the method may add a transfection agent (addition of a transfection agent would be considered to be a condition suitable for transfection). Alternatively, calcium phosphate transfection, electroporation or cationic liposomes could be used. Optionally, the step of transfecting the host cell takes place when the host cell has grown to confluence.

Culturing the host cell under conditions suitable for rAAV production refers to culturing the host cell under conditions at which it can grow and AAV can replicate. For example, the host cell may be cultured at a temperate between 32-C and 40-C, between 34-C and 38-C, between 35-C and 38-C or around 37-C. Optionally, the host cell may be cultured in the presence of a complete cell culture medium such as Dulbecco's Modified Eagle's Medium (DMEM). A completed cell culture medium is a medium that provides all the essential nutrients required for growth of the host cell. Optionally, the complete cell culture medium is supplemented with serum, such as fetal bovine serum or bovine serum albumin.

Optionally, the host cell is a host cell such as those defined above under the heading “host cell”. Optionally, the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell. Optionally, the host cell is a cell that expresses a functional E1A/B protein.

The method may further comprise a step of purifying the rAAV. In general, a step of purifying the rAAV will involve increasing the concentration of the rAAV compared to other components of the preparation. Optionally, the step of purifying the rAAV results in a concentrated rAAV preparation. Optionally, the step of purifying the rAAV results in an isolated rAAV.

Any suitable purification method may be used. Optionally, the step of purifying the rAAV is carried out using a technique selected from the group consisting of gradient density centrifugation (such as CsCl or Iodixanol gradient density centrifugation), filtration, ion exchange chromatography, size exclusion chromatography, affinity chromatography and hydrophobic interaction chromatography.

Optionally, the method comprises further concentrating the rAAV using ultracentrifugation, tangential flow filtration, or gel filtration.

Optionally, the method comprises formulating the rAAV with a pharmaceutically acceptable excipient. The pharmaceutically acceptable excipients may comprise carriers, diluents and/or other medicinal agents, pharmaceutical agents or adjuvants, etc. Optionally, the pharmaceutically acceptable excipients comprise saline solution. Optionally, the pharmaceutically acceptable excipients comprise human serum albumin.

Recombinant AAV (rAAV) Preparations

The present invention further provides a recombinant AAV (rAAV) preparation obtainable by a method of the invention. The invention further provides recombinant AAV preparation obtained by a method of the invention.

The recombinant AAV preparations obtainable or obtained by the methods of the invention are advantageous, as they generally comprise a low level of rcAAV, and/or have a desired ratio of full to total particles. The sections entitled “a desired ratio of full to total particles”, “balance between high yield and high full to total particle ratio” and “a low level of replication competent AAV (rcAAV)” provide further details of what is meant by the terms low level of rcAAV and a desired ratio of full to total particles.

Sequence listing table

| Sequence identity number | Sequence |
|---|---|
| 1 | Genome of AAV2 (AF043303.1) |
| 2 | Genome of AdV5 (AC_000008.1) |
| 3 | Genome of AdV2 (AC_000007.1) |
| 4 | Helper gene region |
| 5 | Alternative AdV5 genome (AY339865.1) |
| 6 | Rep binding site |
| 7 | Rep forward primer |
| 8 | Rep reverse primer |
| 9 | Cap forward primer |
| 10 | Cap reverse primer |

Numbered Aspects of the Invention

1. A two-plasmid system comprising a helper plasmid and a vector plasmid, wherein the helper plasmid comprises at least one rep gene encoding at least one functional Rep protein and does not comprise a cap gene encoding a functional set of Cap proteins.

2. A two-plasmid system comprising a helper plasmid and a vector plasmid, wherein the helper plasmid comprises at least one helper virus gene and does not comprise a cap gene encoding a functional set of Cap proteins, and the at least one helper virus gene is comprised in a contiguous stretch of the plasmid having at least 95%, at least 98%, at least 99%, or 100% identity to the full length or to a fragment at least 6000, at least 7000, or at least 8000 nucleotides in length of SEQ ID NO: 4.

3. The two-plasmid system of aspect 2, wherein the helper plasmid comprises at least one rep gene encoding at least one functional Rep protein.

4. The two-plasmid system of any one of aspects 1-3, wherein the two-plasmid system comprises a molar excess of vector plasmid compared to helper plasmid.

5. The two-plasmid system of any one of aspects 1-4, wherein the ratio of helper plasmid to vector plasmid is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; between 1:1.5 and 1:3; between 1:2 and 1:4; or around 1:3.

6. A helper plasmid comprising at least one rep gene encoding at least one functional Rep protein and at least one helper virus gene, and which does not comprise a cap gene encoding a functional set of Cap proteins.

7. A helper plasmid comprising at least one helper virus gene and which does not comprise a cap gene encoding a functional set of Cap proteins, wherein the at least one helper virus gene is comprised in a contiguous stretch of the plasmid having at least 95%, at least 98%, at least 99%, or 100% identity to the full length or to a fragment at least 6000, at least 7000, or at least 8000 nucleotides in length of SEQ ID NO: 4.

8. The helper plasmid of aspect 7, wherein the helper plasmid comprises at least one rep gene encoding at least one functional Rep protein.

9. A vector plasmid comprising:
(a) a cap gene encoding at least one functional Cap protein; or
(b) at least one cap gene promoter, a cloning site operably linked to the cap gene promoter, and an expression cassette flanked on at least one side by an ITR; wherein the vector plasmid does not comprise a rep gene encoding a functional Rep protein and the expression cassette comprises a transgene operably linked to at least one regulatory control element.

10. The two-plasmid system of any one of aspects 1-5, wherein the vector plasmid comprises:
(a) a cap gene encoding at least one functional Cap protein; or
(b) at least one cap gene promoter, a cloning site operably linked to the cap gene promoter, and an expression cassette flanked on at least one side by an ITR; wherein the vector plasmid does not comprise a rep gene encoding a functional Rep protein and the expression cassette comprises a transgene operably linked to at least one regulatory control element.

11. The two-plasmid system or helper plasmid of any one of aspects 1, 3-6, 8 or 10, wherein the at least one rep gene comprises a gene encoding:
(a) a functional Rep 52 protein;
(b) a functional Rep 40 protein; and/or
(c) a functional Rep 68 protein.

12. The two-plasmid system or helper plasmid of any one of aspects 1, 3-6, 8, 10 or 11, wherein the at least one rep gene comprises a gene encoding a functional Rep 52 protein, at least one gene encoding a functional Rep 40 protein, and a gene encoding a functional Rep 68 protein.

13. The two-plasmid system or helper plasmid of any one of aspects 1-8, or 10-12, wherein the helper plasmid comprises two genes encoding a functional Rep 40 protein.

14. The two-plasmid system or helper plasmid of aspect 13, wherein only one of the two genes encoding a functional Rep 40 protein comprises an intron.

15. The two-plasmid system or helper plasmid of any one of aspects 11-14, wherein the gene encoding a functional Rep 52 protein comprises a nucleic acid sequence having at least 95%, at least 98%, at least 99%, or 100% identity to the full length or a fragment of at least 800, at least 900, at least 1000, or at least 1100 nucleotides in length of nucleotides 993-2186 of SEQ ID NO: 1, or to a corresponding stretch of nucleotides in a different serotype of AAV.

16. The two-plasmid system or helper plasmid any one of aspects 11-15, wherein the at least one gene encoding a functional Rep 40 protein comprises a nucleic acid sequence having at least 95%, at least 98%, at least 99%, or 100% identity to the full length or to a fragment of at least 600, at least 700, at least 800, or at least 900 nucleotides in length of a stretch of nucleotides corresponding to nucleotides 993-2252 minus nucleotides 1907-2227 of SEQ ID NO: 1, or to corresponding stretches of nucleotides in a different serotype of AAV.

17. The two-plasmid system or helper plasmid of any one of aspects 11-16 wherein the at least one gene encoding a functional Rep 40 protein comprises a nucleic acid sequence having at least 95%, at least 98%, at least 99%, or 100% identity to the full length or to a fragment of at least 900, at least 1000, at least 1100, or at least 1200 nucleotides in length of nucleotides 993-2252 of SEQ ID NO: 1, or to a corresponding stretch of nucleotides in a different serotype of AAV.

18. The two-plasmid system or helper plasmid of any one of aspects 11-17, wherein the gene encoding a functional Rep 68 protein comprises a nucleic acid sequence having at least 95%, at least 98%, at least 99%, or 100% identity to the full length or to a fragment of at least 1000, at least 1400, at least 1500, or at least 1600 nucleotides in length of a stretch of nucleotides corresponding to nucleotides 321-2252 minus nucleotides 1907-2227 of SEQ ID NO: 1, or to corresponding stretches of nucleotides in a different serotype of AAV.

19. The two-plasmid system or helper plasmid of any one of aspects 1-8 or 10-18, wherein the helper plasmid does not comprise a gene encoding a functional Rep 78 protein.

20. The two-plasmid system or helper plasmid of any one of aspects 1-8 or 10-19, wherein the helper plasmid does not comprise a contiguous sequence of at least 1700, at least 1800, or 1866 nucleotides corresponding to a contiguous stretch of nucleotides of equivalent length comprised within nucleotides 321-2186 of SEQ ID NO: 1 or within a corresponding stretch of nucleotides in a different serotype of AAV.

21. The two-plasmid system or helper plasmid of any one of aspects 1, 3-6, 8, 10, or 11-20, wherein the at least one rep gene does not comprise a functional internal p40 promoter.

22. The two-plasmid system or helper plasmid of any one of aspects 1, 3-6, 8, 10, or 11-21, wherein the at least one rep gene does not comprise a T nucleotide at a position corresponding to position 1823 of SEQ ID NO: 1.

23. The two-plasmid system or helper plasmid of aspect 22, wherein the at least one rep gene comprises a C nucleotide at a position corresponding to position 1823 of SEQ ID NO: 1.

24. The two-plasmid system or helper plasmid of any one of aspects 1, 3-6, 8, or 11-23, wherein the at least one rep gene does not comprise AAG at positions corresponding to positions 1826-1828 of SEQ ID NO: 1.

25. The two-plasmid system or helper plasmid of any one of aspects 1, 3-6, 8, or 11-24, wherein the at least one rep gene comprises CTC at positions corresponding to positions 1826-1828 of SEQ ID NO: 1.

26. The two-plasmid system or helper plasmid of any one of aspects 1-8 or 10-25, wherein the helper plasmid does not comprise a contiguous stretch of exclusively cap gene sequence of more than 250 nucleotides, more than 100 nucleotides, or more than 60 nucleotides.

27. The two-plasmid system or helper plasmid of aspect 26, wherein the helper plasmid does not comprise a contiguous stretch of exclusively cap gene sequence of more than 60 nucleotides.

28. The two-plasmid system or helper plasmid of any one of aspects 1-8 or 10-27, wherein the helper plasmid comprises a portion of cap gene sequence, and the portion of cap gene sequence does not encode a functional set of Cap proteins.

29. The two-plasmid system or helper plasmid of any one of aspects 1-5 or 10-28, wherein the helper plasmid comprises at least one helper virus gene.

30. The two-plasmid system or helper plasmid of any one of aspects 2, 3, 6-8 or 29, wherein the at least one helper virus gene is an adenovirus gene.

31. The two-plasmid system or helper plasmid of aspect 30, wherein the at least one helper virus gene is an Adenovirus 5 or Adenovirus 2 gene.

32. The two-plasmid system or helper plasmid of any one of aspects 2, 3, 6-8 or 29-31, wherein the least one helper virus gene comprises:
(a) a VA (viral associated) nucleic acid encoding functional VA RNA I and II;
(b) an E2A gene encoding a functional E2A protein; and/or
(c) an E4 gene encoding a functional E4 protein.

33. The two-plasmid system or helper plasmid of aspect 32, wherein the at least one helper virus gene comprises a VA nucleic acid, an E2A gene and an E4 gene.

34. The two-plasmid system or helper plasmid of aspect 33, wherein the E4 gene is not located between the VA nucleic acid and the E2A gene.

35. The two-plasmid system or helper plasmid of any one of aspects 1-8 or 10-34, wherein the helper plasmid is less than 25000 bp, less than 20000 bp, less than 15000 bp, less than 14500 bp, between 10000 bp and 25000 bp, between 10000 bp and 20000 bp, between 12000 bp and 15000 bp, or around 14021 bp in length.

36. The two-plasmid system or helper plasmid of any one of aspects 1-8 or 10-35, wherein the helper plasmid does not comprise a gene encoding a functional adenoviral E1A/B protein.

37. The two-plasmid system or helper plasmid of any one of aspects 1-8 or 10-36, wherein the helper plasmid does not comprise a contiguous sequence of at least 2000, at least 2500, at least 3000, or 3427 nucleotides of a contiguous stretch of nucleotides of equivalent length comprised within nucleotides 194-3620 of SEQ ID NO: 2, or a corresponding stretch of nucleotides in a different serotype of adenovirus.

38. The two-plasmid system or helper plasmid of any one of aspects 1-8 or 10-37, wherein the helper plasmid does not comprise a contiguous sequence of at least 50, at least 60, or 69 nucleotides of a contiguous stretch of nucleotides of equivalent length comprised within nucleotides 4032-4100 of SEQ ID NO: 2, or a corresponding stretch of nucleotides in a different serotype of adenovirus.

39. The two-plasmid system or helper plasmid of any one of aspects 1-8 or 10-38, wherein the helper plasmid does not comprise a contiguous sequence of at least 200, at least 300, at least 350, or 363 nucleotides of a contiguous stretch of nucleotides of equivalent length comprised within nucleotides 4051-4413 of SEQ ID NO: 1, or a corresponding stretch of nucleotides in a different serotype of AAV.

40. The two-plasmid system or helper plasmid of any one of aspects 1-8 or 10-39, wherein the helper plasmid does not comprise a contiguous sequence of at least 400, at least 500, at least 600, or 647 nucleotides of a contiguous stretch of nucleotides of equivalent length comprised within nucleotides 2301-2947 of SEQ ID NO: 1, or a corresponding stretch of nucleotides in a different serotype of AAV.

41. The two-plasmid system or helper plasmid of any one of aspects 32-40, wherein the VA nucleic acid has an activity level which has at least 75%, at least 80%, at least 90%, at least 95%, or between 95% and 100% of the activity of a wild type VA nucleic acid from Adenovirus 5.

42. The two-plasmid system or helper plasmid of aspect 41, wherein the activity level of the VA nucleic acid is determined by measuring recombinant AAV yield.

43. The two-plasmid system or helper plasmid of any one of aspects 32-42, wherein the VA nucleic acid comprises a contiguous sequence at least 95%, at least 98%, or 100% identical to a stretch of at least 15 nucleotides, at least 20 nucleotides, or 25 nucleotides of nucleotides 10595-10619 of SEQ ID NO: 2, or a corresponding stretch of nucleotides in a different serotype of adenovirus.

44. The two-plasmid system or helper plasmid of any one of aspects 32-43, wherein the E2A gene is operably linked to a promoter which comprises a contiguous sequence at least 96%, at least 98%, or 100% identical to a stretch of at least 60, at least 70, at least 80, or 100 nucleotides of nucleotides 27037-27136 of SEQ ID NO: 2, or a corresponding stretch of nucleotides in a different serotype of adenovirus.

45. The two-plasmid system or helper plasmid of any one of aspects 32-44, wherein the at least one helper virus gene comprises a VA nucleic acid, an E2A gene and an E4 gene, and wherein the VA nucleic acid, the E2A gene and the E4 gene are comprised within a contiguous portion of fewer than 15000, fewer than 12000, fewer than 10000, or fewer than 9000 nucleotides.

46. The two-plasmid system or helper plasmid of any one of aspects 1, 3-8 or 10-45, wherein the at least one helper virus gene is comprised on a contiguous stretch of the plasmid having at least 95%, at least 98%, at least 99%, or 100% identity to the full length or to a fragment at least 6000 nucleotides, at least 7000 nucleotides, or at least 8000 nucleotides in length of SEQ ID NO: 4.

47. The two-plasmid system or helper plasmid of any one of aspects 32-46, wherein the helper plasmid does not comprise a contiguous sequence of at least 15000, at least 20000, at least 22000, or 22137 nucleotides of a contiguous stretch of nucleotides of equivalent length comprised within nucleotides 10619-32755 of SEQ ID NO: 2, or a corresponding stretch of nucleotides in a different serotype of adenovirus.

48. The two-plasmid system or helper plasmid of any one of aspects 32-47, wherein the E4 gene is operably linked to an E4 promoter that has at least 50%, at least 70%, or at least 90% of the activity of a wild type promoter from Adenovirus 5.

49. The two-plasmid system or helper plasmid of aspect 48, wherein the E4 promoter comprises a sequence of at least 30, at least 40, or 55 nucleotides corresponding to nucleotides 35793-35848 of SEQ ID NO: 2, or a corresponding stretch of nucleotides in a different serotype of adenovirus.

50. The two-plasmid system, helper plasmid or vector plasmid of any one of aspects 1-49, wherein the helper plasmid and/or the vector plasmid does not comprise an artificial Rep binding site.

51. The two-plasmid system, helper plasmid or vector plasmid of any one of aspects 1-50, wherein the helper plasmid and/or the vector plasmid comprises a plasmid backbone, and the plasmid backbone does not comprise an artificial Rep binding site.

52. The two-plasmid system or vector plasmid of any one of aspects 1-5, or 9-51, wherein the vector plasmid comprises a cap gene operably linked to at least one cap gene promoter.

53. The two-plasmid system or vector plasmid of any one of aspects 1-5, or 9-52, wherein the vector plasmid comprises a cap gene and further comprises an expression cassette flanked on at least one side by an ITR.

54. The two-plasmid system or vector plasmid of any one of aspects 1-5 or 9-53, wherein the vector plasmid comprises a cap gene and the cap gene encodes a VP1, a VP2, and/or a VP3 protein.

55. The two-plasmid system or vector plasmid of any one of aspects 1-5 or 9-54, wherein the vector plasmid comprises a cap gene and the cap gene encodes a Cap protein selected from the group of AAV serotypes consisting of serotypes 1, 2, 3A, 3B, 4, 5, 6, 7, 8, 9, 10, 11, 12, or 13, LK03, rh74, rh10, and Mut C (SEQ ID NO: 3 from WO 2016/181123).

56. The two-plasmid system or vector plasmid of any one of aspects 1-5 or 9-55, wherein the vector plasmid comprises a cap gene and the cap gene encodes a Cap protein selected from the group of AAV serotypes consisting of serotypes 2, 5, 8, 9, and Mut C (SEQ ID NO: 3 from WO 2016/181123).

57. The two-plasmid system or vector plasmid of any one of aspects 1-5 or 9-56, wherein the vector plasmid comprises an at least one cap gene promoter, which is a native cap gene promoter.

58. The two-plasmid system or vector plasmid of any one of aspects 1-5 or 9-57, wherein the vector plasmid comprises an at least one cap gene promoter, which comprises an AAV p40 promoter, a p5 promoter, and/or a p19 promoter.

59. The two-plasmid system or vector plasmid of aspect 58, wherein the at least one cap gene promoter comprises a p40 promoter.

60. The two-plasmid system or vector plasmid of aspect 58 or 59, wherein the at least one cap gene promoter comprises a p40 promoter, a p5 promoter, and a p19 promoter.

61. The two-plasmid system or vector plasmid of any one of aspects 9-60, wherein the transgene encodes an enzyme, a metabolic protein, a signalling protein, an antibody, an antibody fragment, an antibody-like protein, an antigen, or a non-translated RNA such as an miRNA, siRNA, snRNA, or antisense RNA.

62. The two-plasmid system or vector plasmid of any one of aspects 9-61, wherein the transgene encodes a protein selected from the group consisting of Factor IX, α-Galactosidase A, beta-Glucocerebrosidase and Factor VIII.

63. The two-plasmid system or vector plasmid of any one of aspects 9-62, wherein the cloning site is a multi-cloning site (MCS).

64. The two-plasmid system or vector plasmid of any one of aspects 1-5 or 9-63, wherein the vector plasmid does not comprise any dispensable translation initiation codons.

65. The two-plasmid system or vector plasmid of aspect 64, wherein the vector plasmid comprises a promoter region comprising one or more promoters, and the promoter region does not comprise ATG or GTG codons.

66. The two-plasmid system or vector plasmid of aspect 65, wherein the promoter region comprises p5, p19 and p40 promoters, and wherein ATG or GTG codons at one or more positions corresponding to positions (a) 321-323, (b) 766-768, (c) 955-957, (d) 993-995 and (e) 1014-1016 of SEQ ID NO: 1 are absent or mutated.

67. The two-plasmid system or vector plasmid of aspect 66, wherein in the promoter region:
(a) nucleotides corresponding to nucleotides 321-323 of SEQ ID NO: 1 are absent;
(b) nucleotides corresponding to nucleotides 766-768 of SEQ ID NO: 1 are not ATG and are optionally ATT;
(c) nucleotides corresponding to nucleotides 955-957 of SEQ ID NO: 1 are absent;
(d) nucleotides corresponding to nucleotides 993-995 of SEQ ID NO: 1 are absent; and/or
(e) nucleotides corresponding to nucleotides 1014-1016 of SEQ ID NO: 1 are absent.

68. The two-plasmid system or vector plasmid of aspect 67, wherein in the promoter region:
(a) nucleotides corresponding to nucleotides 321-323 of SEQ ID NO: 1 are absent;
(b) nucleotides corresponding to nucleotides 766-768 of SEQ ID NO: 1 are not ATG and are optionally ATT;
(c) nucleotides corresponding to nucleotides 955-957 of SEQ ID NO: 1 are absent;
(d) nucleotides corresponding to nucleotides 993-995 of SEQ ID NO: 1 are absent; and
(e) nucleotides corresponding to nucleotides 1014-1016 of SEQ ID NO: 1 are absent.

69. The two-plasmid system or vector plasmid of any one of aspects 1-5 or 9-68, wherein the vector plasmid comprises a backbone less than 4000 nucleotides, less than 3500 nucleotides, less than 3000 nucleotides, or less than 2500 nucleotides in length.

70. The two-plasmid system or vector plasmid of any one of aspects 1-5 or 9-69, wherein the vector plasmid does not comprise any spacers.

71. A host cell comprising the two-plasmid system, the helper plasmid or the vector plasmid of any one of the preceding aspects.

72. Use of the two-plasmid system, the helper plasmid or the vector plasmid as defined in any one of aspects 1-70 for producing a recombinant AAV preparation:
(a) having a low level of replication competent AAV (rcAAV);
(b) having a desired ratio of full to total particles; and/or
(c) at a high or desired yield.

73. The use of aspect 72, wherein the low level of rcAAV comprises a low level of rep-rcAAV.

74. The use of aspect 72 or 73, wherein the low level of rcAAV comprises a lower level of rep-rcAAV compared to the level of rep-rcAAV produced using a two-plasmid system comprising a plasmid comprising both at least one rep gene and at least one cap gene.

75. The use of any one of aspects 72-74, wherein the low level of rcAAV comprises an at least 5 times, at least 10 times, at least 25 times, at least 30 times, or at least 35 times excess of rep-rcAAV or cap-rcAAV.

76. The use of any one of aspects 72-75, wherein the low level of rcAAV comprises a proportion of less than ⅕ rep-rcAAV, less than 1/10 rep-rcAAV, less than 1/25 rep-rcAAV, less than 1/30 rep-rcAAV, or less than 1/35 rep-rcAAV.

77. The use of any one of aspects 72-76, wherein the low level of rcAAV comprises a proportion of rep-rcAAV that is less than the proportion produced using a two-plasmid system comprising a plasmid comprising both at least one rep gene and at least one cap gene.

78. The use of aspect 77, wherein the proportion of rep-rcAAV is less than 90%, less than 75%, less than 60%, less than 50%, less than 25%, less than 20%, less than 17%, or less than 15% of the proportion produced using the two-plasmid system comprising a plasmid comprising both at least one rep gene and at least one cap gene.

79. The use of any one of aspects 72-78, wherein the low level of rcAAV comprises a low level of pseudo-wild type rcAAV.

80. The use of any one of aspects 72-79, wherein the low level of rcAAV comprises less than ⅕ pseudo-wild type rcAAV, less than 1/10 pseudo-wild type rcAAV, less than 1/25 pseudo-wild type rcAAV, less than 1/30 pseudo-wild type rcAAV, or less than 1/35 pseudo-wild type rcAAV.

81. The use of any one of aspects 72-80, wherein the level of rep-rcAAV is the level of rep-rcAAV detected by qPCR using primers binding to rep68 exon 1.

82. The use of any one of aspects 72-81, wherein the level of rep-rcAAV is the level of rep-rcAAV detected by qPCR using the forward primer CACGTGCATGTGGAAGTAG (SEQ ID NO: 7) and the reverse primer CGACTTTCTGACGGAATGG (SEQ ID NO: 8).

83. The use of any one of aspects 72-82, wherein the level of cap-rcAAV is the level of cap-rcAAV detected by qPCR using primers binding to a sequence encoding VP3.

84. The use of any one of aspects 72-83, wherein the level of cap-rcAAV is the level of cap-rcAAV detected by qPCR using the forward primer TACTGAGGGACCATGAAGAC (SEQ ID NO: 9) and the reverse primer GTTTACGGACTCGGAGTATC (SEQ ID NO: 10).

85. Use of the two-plasmid system, the helper plasmid or the vector plasmid as defined in any one of aspects 1-70 for:
(a) reducing or minimising the level of replication competent AAV (rcAAV) produced during recombinant AAV production;
(b) reducing or minimising the level of pseudo-wild type replication competent AAV (rcAAV) produced during recombinant AAV production;
(c) controlling or maximising the ratio of full to total particles produced during recombinant AAV production; and/or
(d) increasing, optimising or maximising the yield of recombinant AAV produced during recombinant AAV production.

86. The use of any one of aspects 72-85, wherein the use comprises transfecting a host cell with the two-plasmid system, the helper plasmid or the vector plasmid of any one of aspects 1-70 and culturing the host cell under conditions suitable for recombinant AAV production.

87. The use of aspect 85 or 86, wherein an excess of rep-rcAAV or cap-rcAAV indicates that the level of pseudo wild-type rcAAV is reduced or minimised.

88. The use of aspect 87, wherein the excess is at least 5 times, at least 10 times, at least 25 times, at least 30 times, or at least 35 times excess of rep-rcAAV or cap-rcAAV.

89. The use of aspect 87 or 88, wherein the excess is greater than the excess produced using a two-plasmid system comprising a plasmid comprising both at least one rep gene and at least one cap gene.

90. The use of any one of aspects 87-89, wherein the level of rep-rcAAV is the level of rep-rcAAV detected by qPCR using primers binding to rep68 exon 1.

91. The use of any one of aspects 87-90, wherein the level of rep-rcAAV is the level of rep-rcAAV detected by qPCR using the forward primer CACGTGCATGTGGAAGTAG (SEQ ID NO: 7) and the reverse primer CGACTTTCTGACGGAATGG (SEQ ID NO: 8).

92. The use of any one of aspects 87-91, wherein the level of cap-rcAAV is the level of cap-rcAAV detected by qPCR using primers binding to a sequence encoding VP3.

93. The use of any one of aspects 87-92, wherein the level of cap-rcAAV is the level of cap-rcAAV detected by qPCR using the forward primer TACTGAGGGACCATGAAGAC (SEQ ID NO: 9) and the reverse primer GTTTACGGACTCGGAGTATC (SEQ ID NO: 10).

94. A method for producing a recombinant AAV preparation comprising:
(a) obtaining the two-plasmid system, the helper plasmid or the vector plasmid as defined in any one of aspects 1-70;
(b) transfecting a host cell with the two-plasmid system, the helper plasmid or the vector plasmid as defined in any one of aspects 1-70; and
(c) culturing the host cell under conditions suitable for recombinant AAV production.

95. The method of aspect 94, further comprising a step of harvesting the recombinant AAV to provide a recombinant AAV preparation.

96. A method for reducing or minimising the level of replication competent AAV (rcAAV) produced during recombinant AAV production comprising:
(a) obtaining the two-plasmid system, the helper plasmid or the vector plasmid as defined in any one of aspects 1-70;
(b) transfecting a host cell with the two-plasmid system, the helper plasmid or the vector plasmid as defined in any one of aspects 1-70; and
(c) culturing the host cell under conditions suitable for recombinant AAV production.

97. A method for reducing or minimising the level of pseudo-wild type replication competent AAV (rcAAV) produced during recombinant AAV production comprising:
(a) obtaining the two-plasmid system, the helper plasmid or the vector plasmid as defined in any one of aspects 1-70;
(b) transfecting a host cell with the two-plasmid system, the helper plasmid or the vector plasmid as defined in any one of aspects 1-70; and
(c) culturing the host cell under conditions suitable for recombinant AAV production.

98. The method of aspect 97, wherein an excess of rep-rcAAV or cap-rcAAV indicates that the level of pseudo wild-type rcAAV is reduced or minimised.

99. The method of aspect 98, wherein the excess is at least 5 times, at least 10 times, at least 25 times, at least 30 times, or at least 35 times excess of rep-rcAAV or cap-rcAAV 100. The method of aspect 98 or 99, wherein the excess is greater than the excess produced using a two-plasmid system comprising a plasmid comprising both at least one rep gene and at least one cap gene.

101. The method of any one of aspects 98-100, wherein the level of rep-rcAAV is the level of rep-rcAAV detected by qPCR using primers binding to rep68 exon 1. 102. The method of any one of aspects 98-101, wherein the level of rep-rcAAV is the level of rep-rcAAV detected by qPCR using the forward primer CACGTGCATGTGGAAGTAG (SEQ ID NO: 7) and the reverse primer CGACTTTCTGACGGAATGG (SEQ ID NO: 8).

103. The method of any one of aspects 98-102, wherein the level of cap-rcAAV is the level of cap-rcAAV detected by qPCR using primers binding to a sequence encoding VP3.

104. The method of any one of aspects 98-103, wherein the level of cap-rcAAV is the level of cap-rcAAV detected by qPCR using the forward primer TACTGAGGGACCATGAAGAC (SEQ ID NO: 9) and the reverse primer GTTTACGGACTCGGAGTATC (SEQ ID NO: 10).

105. The use or method of aspect 86-93 or 96-104, further comprising a step of harvesting the recombinant AAV to provide a recombinant AAV preparation.

106. The use or method of any one of aspects 94, 95 or 105, wherein the recombinant AAV preparation comprises a low level of rcAAV.

107. The use or method of aspect 106, wherein the low level of rcAAV comprises a low level of rep-rcAAV.

108. The use or method of aspect 106 or 107, wherein the low level of rcAAV comprises a lower level of rep-rcAAV compared to the level of rep-rcAAV produced using a two-plasmid system comprising a plasmid comprising both at least one rep gene and at least one cap gene.

109. The use or method of any one of aspects 106-108, wherein the low level of rcAAV comprises an at least 5 times, at least 10 times, at least 25 times, at least 30 times, or at least 35 times excess of rep-rcAAV or cap-rcAAV.

110. The use or method of any one of aspects 106-109, wherein the low level of rcAAV comprises a proportion of less than ⅕ rep-rcAAV, less than 1/10 rep-rcAAV, less than 1/25 rep-rcAAV, less than 1/30 rep-rcAAV, or less than 1/35 rep-rcAAV.

111. The use or method of any one of aspects 106-110, wherein the low level of rcAAV comprises a proportion of rep-rcAAV that is less than the proportion produced using a two-plasmid system comprising a plasmid comprising both at least one rep gene and at least one cap gene.

112. The use or method of aspect 111, wherein the proportion of rep-rcAAV is less than 90%, less than 75%, less than 60%, less than 50%, less than 25%, less than 20%, less than 17%, or less than 15% of the proportion produced using the two-plasmid system comprising a plasmid comprising both at least one rep gene and at least one cap gene.

113. The use or method of any one of aspects 106-112, wherein the low level of rcAAV comprises a low level of pseudo-wild type rcAAV.

114. The use or method of any one of aspects 106-113, wherein the low level of rcAAV comprises less than ⅕ pseudo-wild type rcAAV, less than 1/10 pseudo-wild type rcAAV, less than 1/25 pseudo-wild type rcAAV, less than 1/30 pseudo-wild type rcAAV, or less than 1/35 pseudo-wild type rcAAV.

115. The use or method of any one of aspects 106-114, wherein the level of rep-rcAAV is the level of rep-rcAAV detected by qPCR using primers binding to rep68 exon 1.

116. The use or method of any one of aspects 106-115, wherein the level of rep-rcAAV is the level of rep-rcAAV detected by qPCR using the forward primer CACGTGCATGTGGAAGTAG (SEQ ID NO: 7) and the reverse primer CGACTTTCTGACGGAATGG (SEQ ID NO: 8).

117. The use or method of any one of aspects 106-116, wherein the level of cap-rcAAV is the level of cap-rcAAV detected by qPCR using primers binding to a sequence encoding VP3.

118. The use or method of any one of aspects 106-117, wherein the level of cap-rcAAV is the level of cap-rcAAV detected by qPCR using the forward primer TACTGAGGGACCATGAAGAC (SEQ ID NO: 9) and the reverse primer GTTTACGGACTCGGAGTATC (SEQ ID NO: 10).

119. The use or method of any one of aspect 72-84 or 106-118, wherein the level of rcAAV is measured to be less than 1 rcAAV in 10⁷ recombinant AAV, less than 1 rcAAV in 10⁹ recombinant AAV, or less than 1 rcAAV in 10¹⁰ recombinant AAV.

120. The use or method of any one of aspect 106-119, wherein the level of rcAAV is less than the level of rcAAV produced using an equivalent method except that the vector plasmid comprises both at least one rep gene and at least one cap gene.

121. The use or method of any one of aspects 72-84, 86-93 or 106-120, wherein the level of rcAAV is measured by using qPCR to measure the number of recombinant AAV particles that comprise a rep gene.

122. The use or method of any one of aspects 72-84, 86-95 or 105-121, wherein the recombinant AAV preparation has a desired ratio of full to total particles.

123. A method for controlling or maximising the ratio of full to total particles produced during recombinant AAV production comprising:
(a) obtaining the two-plasmid system, the helper plasmid or the vector plasmid as defined in any one of aspects 1-70;
(b) transfecting a host cell with the two-plasmid system, the helper plasmid or the vector plasmid as defined in any one of aspects 1-70; and
(c) culturing the host cell under conditions suitable for recombinant AAV production.

124. The method of aspect 123, further comprising a step of harvesting the recombinant AAV to provide a recombinant AAV preparation comprising a desired ratio of full to total particles.

125. The use or method of any one of aspects 72-84, 86-93, 122, or 124, wherein the desired ratio of full to total particles is at least 2%, at least 3%, at least 4%, at least 5%, at least 7%, at least 10% or at least 15%.

126. The method of any one of aspects 94-125, wherein the method is a method for producing a recombinant AAV preparation at a high or desired yield.

127. A method for increasing, optimising or maximising the yield of recombinant AAV produced during recombinant AAV production comprising:
(a) obtaining the two-plasmid system, the helper plasmid or the vector plasmid as defined in any one of aspects 1-70;
(b) transfecting a host cell with the two-plasmid system, the helper plasmid or the vector plasmid as defined in any one of aspects 1-70; and
(c) culturing the host cell under conditions suitable for recombinant AAV production.

128. The method of aspect 127, further comprising a step of harvesting the recombinant AAV to provide a recombinant AAV preparation comprising a high or desired yield of recombinant AAV.

129. The use or method of any one of aspects 72-84, 86-93, 124-126 or 128, wherein the ratio of helper plasmid to vector plasmid is adjusted to obtain the desired ratio of full to total particles and/or the high or desired yield of recombinant AAV.

130. The use or method of any one of aspects 72-84, 86-93, or 123-129, comprising a step of selecting a ratio of helper plasmid to vector plasmid.

131. The use or method of aspect 130, wherein the ratio of helper plasmid to vector plasmid is selected or adjusted to a ratio that allows the user to obtain the desired ratio of full to total particles or the high or desired yield of recombinant AAV.

132. The use or method of any one of aspects 72-93, 122-126 or 128-131, wherein the ratio of helper plasmid to vector plasmid is selected or adjusted to a ratio that achieves a balanced yield versus full to total particle ratio.

133. The use or method of any one of aspects 72-93, 122-126 or 128-132, wherein the ratio of helper plasmid to vector plasmid is selected or adjusted to a ratio that achieves a maximum yield of recombinant AAV with the minimum yield of empty particles achievable at such maximum yield of recombinant AAV.

134. The use or method of any one of aspects 72-84, 86-93, 125, 126 or 128-133, wherein the high or desired yield is a yield that is at least 2-fold, at least 4-fold, at least 5-fold, or at least 6-fold higher than the yield achieved using an equivalent method with a ratio of helper plasmid to vector plasmid of 1.8:1.

135. The use or method of any one of aspects 72-84, 86-93, 125, 126 or 128-134, wherein the high or desired yield is a yield greater than 2×10¹⁰ vg/ml, greater than 4×10¹⁰ vg/ml, greater than 6×10¹⁰ vg/ml, greater than 8×10¹⁰ vg/ml, greater than 1×10¹¹ vg/ml, greater than 2×10¹¹ vg/ml, or greater than 4×10¹¹ vg/ml.

136. The use or method of any one of aspects 72-84, 86-93, 122, 124-126 or 129-135, wherein the desired ratio of full to total particles is a ratio of full to total particles that is at least 20% or at least 30% of the ratio of full to total particles achieved using an equivalent method with a ratio of helper plasmid to vector plasmid of 1.8:1.

137. The use or method of any one of aspects 72-84, 86-93, 122-126 or 129-136, wherein the ratio of full to total particles is measured by calculating the number of vector genomes using qPCR to quantify the number of nucleic acid sequences comprising a cassette comprising a promoter sequence (vector genomes; vg); measuring the total number of particles using a capsid-specific ELISA; and calculating the ratio using the formula vg/ml÷particles/ml×100.

138. The use or method of any one of aspects 72-84, 86-93, 123-126 or 128-137, wherein the yield of recombinant AAV is determined by using qPCR to quantify the number of nucleic acid sequences comprising a cassette comprising a promoter sequence (vg).

139. The use or method of any one of aspects 72-93, 122-126 or 128-138, wherein the ratio of helper plasmid to vector plasmid is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; between 1:1.5 and 1:3; between 1:2 and 1:4; or around 1:3.

140. The use or method of any one of aspects 72-93, 122-126 or 128-139, wherein the ratio of helper plasmid to vector plasmid comprises a molar excess of vector plasmid compared to helper plasmid.

141. The method or use of any one of aspects 72-140, wherein the host cell is selected from the group consisting of a HEK293T cell, a HEK293 cell, a HEK293EBNA cell, a CAP cell, a CAP-T cell, an AGE1.CR cell, a PerC6 cell, a C139 cell, and an EB66 cell.

142. The method or use of any one of aspects 72-141, wherein the host cell is a cell that expresses a functional E1A/B protein.

143. The method or use of any one of aspects 72-142, further comprising a step of purifying the recombinant AAV particles.

144. The method or use of aspect 143, where the step of purifying the recombinant AAV particles is carried out using a technique selected from the group consisting of gradient density centrifugation (such as CsCl or Iodixanol gradient density centrifugation), filtration, ion exchange chromatography, size exclusion chromatography, affinity chromatography and hydrophobic interaction chromatography.

145. The method or use of aspect 143 or 144, comprising further concentrating the recombinant AAV using ultracentrifugation, tangential flow filtration, or gel filtration.

146. The method or use of any one of aspects 72-145, comprising formulating the recombinant AAV with a pharmaceutically acceptable excipient.

147. A recombinant AAV preparation obtainable by the method of any one of aspects 94-146.

148. A recombinant AAV preparation obtained by the method of any one of aspects 94-146.

### EXAMPLES

### Example 1—Construction of Helper and Vector Plasmids of (‘Trans-Split’) Two-Plasmid System

Helper Plasmid

To make the helper plasmid, nucleotides 200-4497 of wild type AAV2 (Genbank accession number AF043303; SEQ ID NO: 1) containing the rep and cap genes were cloned into the pUC19 (Yanisch-Perron et al (1985), Gene, 33:103-119). Next, AAV2 nucleotides 4461-4497 were deleted to minimise sequence homology with the vector plasmid (the construction of which is described below). To prevent Rep 78 expression while maintaining Rep 68 expression, the intron within the cloned rep gene was deleted. In order to provide for expression of Rep 52 following deletion of the intron, AAV2 nucleotides corresponding to rep 52 including the p19 promoter were cloned immediately 3′ of the intron-less rep 68 gene. The majority of cap gene sequences were then deleted.

The two p40 promoters (one in each of the Rep 68- and Rep 52-encoding rep gene duplications) were rendered non-functional by ablation of the TATA boxes (mutation of the T corresponding to AAV2 position 1823 and AAG corresponding to AAV2 positions 1826-1828 to C and CTC respectively).

The resulting ‘rep cassette’ was then cloned into a plasmid comprising an 8342-nucleotide stretch comprising functional VA RNA I and II, E2A and E4 genes from adenovirus (i.e. helper virus) serotype 5 (SEQ ID NO: 4). The plasmid backbone, containing kanamycin resistance gene and bacterial origin of replication, was about 2.2 kb in length, resulting in a helper plasmid of 14021 nucleotides.

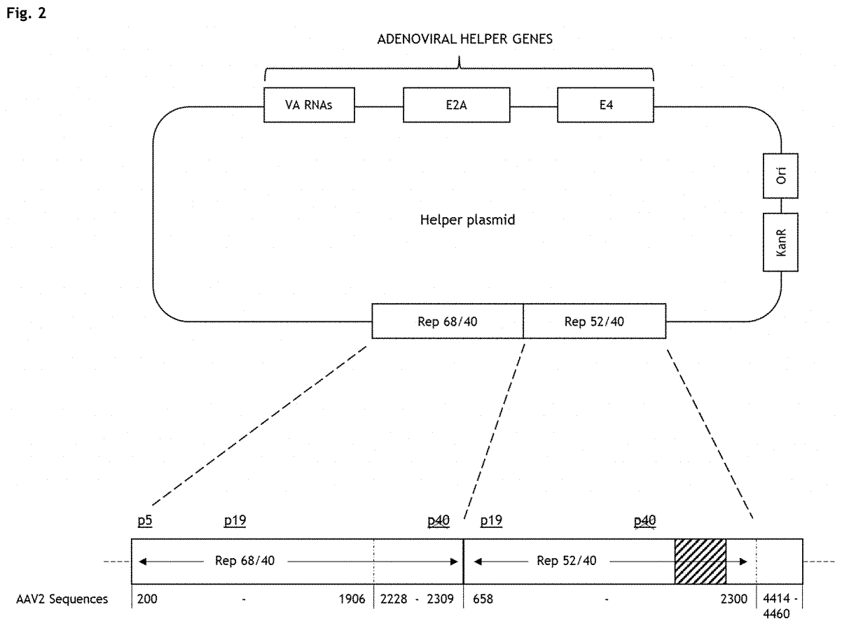

FIG. 2 is a schematic of the helper plasmid showing the main features. For the ‘rep cassette’ the portions of AAV2 sequence, by reference to the nucleotide positions of SEQ ID NO: 1, are indicated.

Vector Plasmid

To make the vector plasmid, nucleotides 200-4497 of wild type AAV2 (Genbank accession number AF043303; SEQ ID NO: 1) containing the rep and cap genes were cloned into the pUC19. Two portions of rep gene sequence, between the p5 and p19 and between the p19 and p40 promoters respectively, were then deleted to prevent Rep protein expression whilst maintaining the downstream cap gene under the regulation of the three native promoters. As for the helper plasmid described above, AAV2 nucleotides 4461-4497 were deleted to minimise sequence homology with the helper plasmid.

To minimise or prevent translation of undesired products from potential initiation codons within the remaining promoter region, four ATG codons and one GTG codon were removed: ATGs at positions corresponding to AAV2 nucleotides 321-323, 955-957 and 993-995, and a GTG corresponding to AAV2 nucleotides 1014-1016, were deleted, whilst ATG at AAV2 nucleotides 766-768 was mutated to ATT.

The AAV2 cap gene encoding the VPs 1, 2 and 3 immediately 3′ of the above promoter region was replaced with the corresponding sequence of an engineered cap gene, which cap gene is 3 nucleotides (equating to one additional encoded amino acid) longer than the AAV2 cap gene. The resulting ‘promoter-cap’ cassette was cloned into a plasmid backbone containing kanamycin resistance gene and bacterial origin of replication. Into this backbone was inserted an expression cassette, containing the transgene sequence linked to promoter and polyA transcription regulatory elements, flanked by AAV2 sequence comprising the native AAV2 ITRs (AAV2 nucleotides 1-145 and 4535-4679).

In the case of vector plasmid comprising the 2672-nucleotide (ITR-to-ITR) Factor IX expression cassette, as used in Example 2, the plasmid backbone was approximately 2.3 kb in length, resulting in a 8525-nucleotide vector plasmid. In all subsequent work, including production of the rAAV batches of Example 3, the plasmid backbone was reduced in length to approximately 2 kb. The α-galactosidase A (GLA) expression cassette (Example 3) was 2297 nucleotides in length.

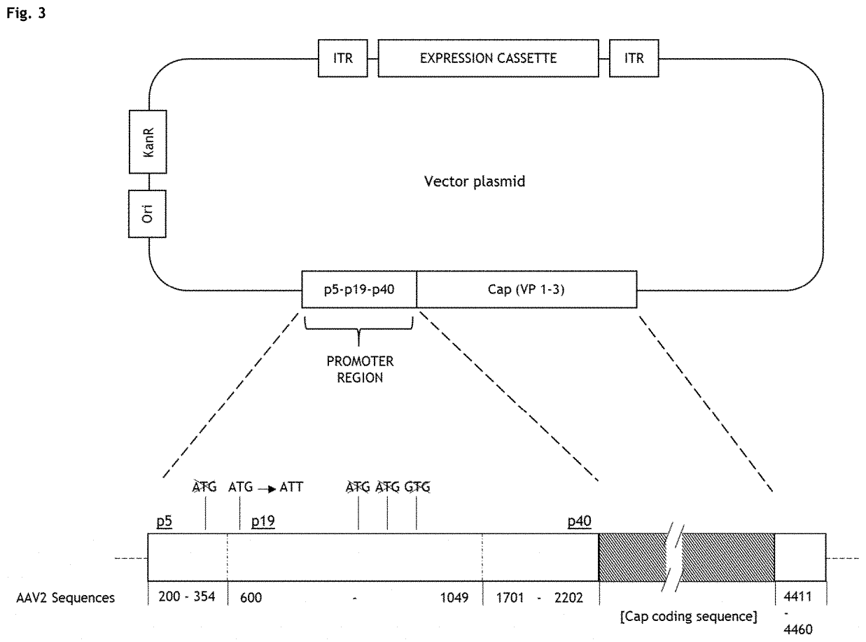

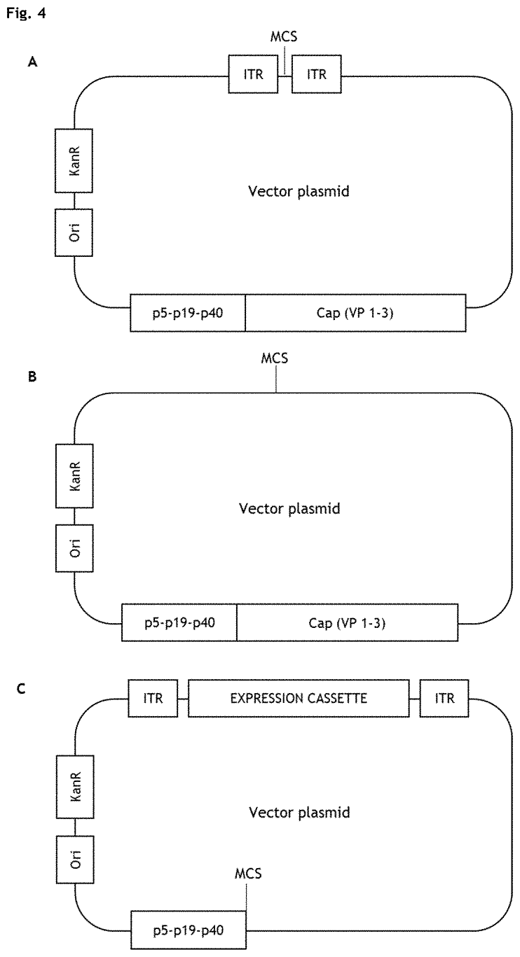

FIG. 3 is a schematic of the vector plasmid showing the main features. For the ‘promoter-cap’ cassette, the portions of AAV2 sequence, by reference to the nucleotide positions of SEQ ID NO: 1, are indicated.

### Example 2—Two-Plasmid System: Comparison of Helper:Vector Plasmid Ratios

Cell Cultivation

HEK293T cells were maintained in adherent culture under standard conditions at 37° C., 95% relative humidity, and 5% v/v CO₂ in Dulbecco's Modified Eagle's Medium (DMEM) supplemented with 10% Fetal Bovine Serum (FBS) and 1% GlutaMax™ (L-alanine-L-glutamine dipeptide). Cellular confluence during passaging ranged from 40-95%.

Transfection of HEK293T Cells and Preparation of Cell Lysates

HEK293T cells were transfected with helper plasmid and vector plasmid (the latter comprising the engineered cap gene and Factor IX expression cassette; Example 1) using different molar plasmid ratios (from helpervector 3:1 to 1:6) while maintaining the total plasmid DNA amount. 1.5×10⁵ viable cells per cm² culture area were seeded in 6-cm dishes in a volume of 3 ml DMEM, 10% FBS, 1% GlutaMax™ the day before transfection resulting in 60-70% confluency on the day of transfection. PEI-DNA complexes were prepared in DMEM without supplements using the linear polyethylenimine transfection reagent PEIpro™ (Polyplus) according to the manufacturer's manual. An amount of 6 μg total plasmid DNA and a PEI-to-DNA ratio of 2:1 was maintained independent of the applied plasmid combination ratios. Cells were cultured until day 3 post-transfection, harvested in the medium and lysed by three freeze-thaw cycles (−80° C. and 37° C.). Cell debris was removed by centrifugation at 10,000×g for 5 min.

Quantification of rAAV Vector Genomes

The AAV vector genome assay is based on a quantitative polymerase chain reaction (qPCR) specific for the promoter sequence of the rAAV expression cassette. In principle, the qPCR primers can be designed to bind any part of the recombinant AAV genome which is not common to wild type AAV genomes, but is it recommended against using primer template sequences very close to the ITRs as doing so can lead to an exaggerated vector genome titre measurement.

Cell lysate test samples were subjected to a nuclease treatment procedure in order to remove non-packed vector genomes prior to performing the qPCR. To that aim the samples were pre-diluted 1:250 in nuclease-free water containing 0.125% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, a trending control containing unpurified cell lysate with a known AAV vector genome titre and spike-in controls using plasmid DNA carrying the promoter sequence were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with following program steps: 95° C. 5 min; 39 cycles (95° C. 10 s, 60° C. 30 s, plate read); 95° C. 10 sec; 60-95° C. (+0.5° C./step), 10 sec; plate read. To control for the quality of the qPCR, a trending control with known AAV vector genome titre was measured in parallel. To check for contaminations, a no template control (NTC, 5 μl H₂O) was also included. Standard row, test samples and controls were measured in triplicates for each dilution. Purified virus test samples and trending control were generally measured in 3 different dilutions in EB buffer (10 mM Tris-Cl, pH 8.5). Turbo DNase treated cell lysate test samples were directly used in the qPCR without any further dilution. Data were analysed using the CFX Manager™ Software 3.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the promoter genetic material, in the form of a linearised plasmid, were serially diluted to create a standard curve and sample vector genome titre was interpolated from the standard curve.

Quantification of rAAV Particles (Capsids)

The AAV2 Titration ELISA method is a measure of total AAV particles (capsids) and is based on a commercially available kit (Progen™, Heidelberg, Germany; catalogue number PRATV). This sandwich immunometric technique utilises monoclonal antibody A20 (Wobus et al (2000), J Virol, 74:9281-9293) for both capture and detection. The antibody is specific for a conformational epitope present on assembled capsids of serotypes AAV2, AAV3, and the engineered capsid used in these experiments.

The AAV2 Titration ELISA kit was used to quantify total AAV particles in cell lysates and purified virus preparations according to the manufacturer's instructions. In brief, 100 μl diluted AAV2 Kit Control, test samples, or trending control of engineered capsid with known total particle titre were added per well of a microtiter plate coated with monoclonal antibody A20 and incubated for 1 h at 37° C. Standard row, test samples and controls were measured in duplicates for each dilution. In a second step, 100 μl of pre-diluted biotin-conjugated monoclonal antibody A20 (1:20 in Assay Buffer [ASSB]) were added and incubated for 1 h at 37° C. Then, 100 μl of a pre-diluted streptavidin peroxidase conjugate (1:20 in ASSB) were added and incubated for 1 h at 37° C. 100 μl substrate solution (TMB [Tetramethylbenzidine]) were added and after incubation for 15 min, the reaction was stopped using 100 μl stop solution. The absorbance was measured photochemically at 450 nm using the SpectraMax M3 microplate reader (Molecular Devices, San Jose, USA). Data was analysed with the SoftMax Pro 7.0 Software (Molecular Devices).

The test samples were diluted into the assay range and AAV total particle concentrations were determined by interpolation using the standard curve which was prepared using the provided AAV2 Kit Control. ASSB was used as blank.

Vector Genome to Total Particle Ratio

The ratio of vector genomes to total AAV particles is expressed as a percentage. This is based on the vector genome titre (determined by qPCR, as described above) and the number of total AAV particles (determined by the capsid ELISA, as described above).

Results

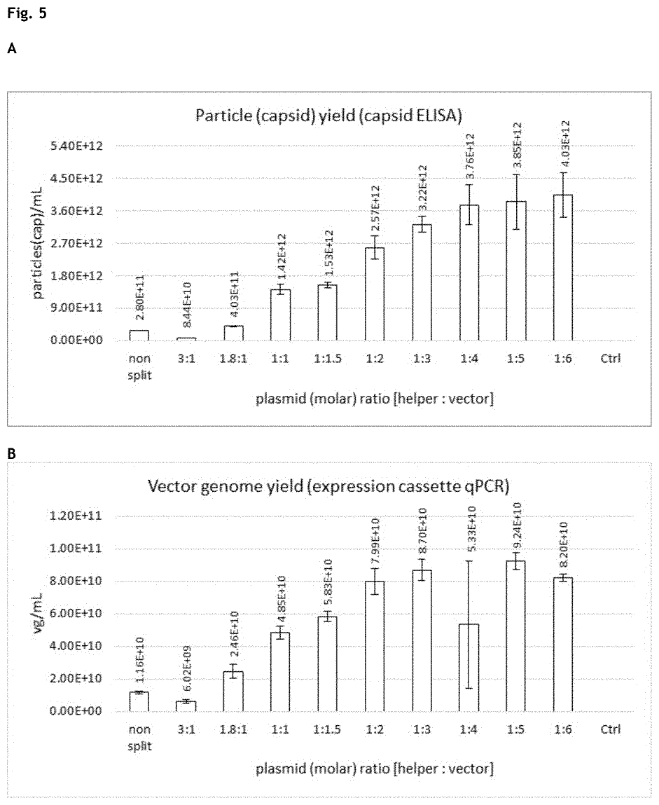

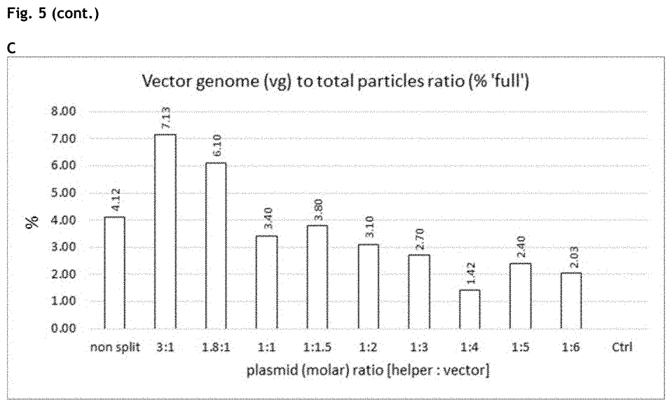

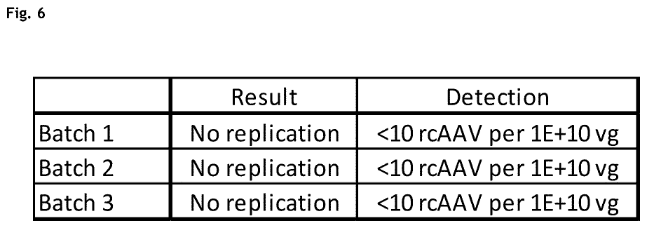

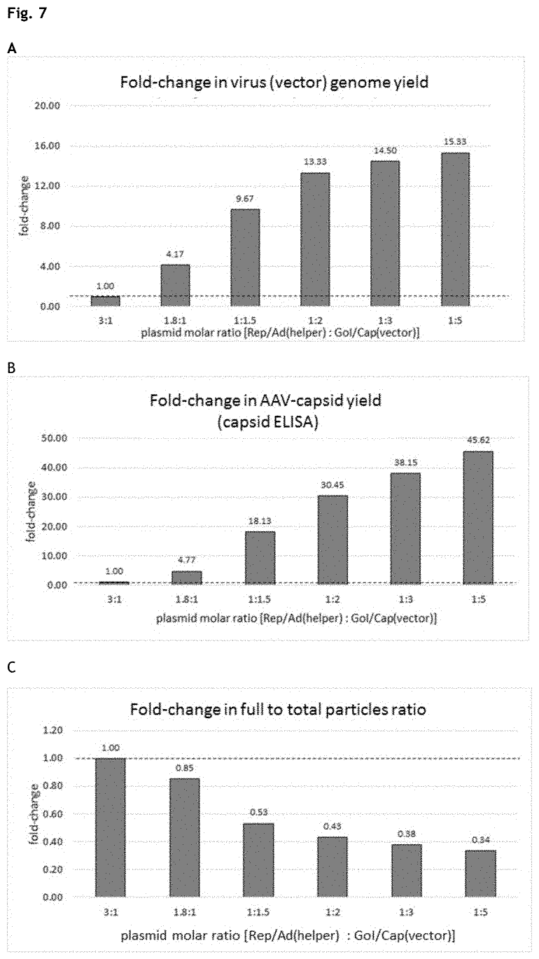

As apparent from FIGS. 5A and B, elevation of the proportion of vector plasmid led to both higher particle and vector genome yields. However, the relative increase in particle (capsid) yields was more pronounced and the plateau in vector genome yields was achieved earlier. These observations were responsible for a gradual decrease in the vector genome to total particles ratio (FIGS. 5C and 7C). A plasmid ratio of helpervector 1:3 resulted in an almost maximal increase in vector genome titre of approximately 3.5-fold compared to the previously applied 1.8:1 ratio (FIG. 7B), whereas the particle titer was increased approximately 8-fold (FIG. 7A).

For comparison, two plasmids in a conventional “non-split” configuration (i.e. wherein one plasmid contained the same AdV helper and vector genome (Factor IX expression cassette) sequences, and the other plasmid contained the same cap gene sequence in addition to an AAV2 rep cassette containing all four rep genes such that Rep and Cap functions are not split between the two plasmids) were transfected in an established molar plasmid ratio of 1.6:1, revealing considerably lower particle and vector genome yields.

Subsequent confirmatory sequencing of the helper plasmid revealed a mutation in the codon within the Rep 68-encoding sequence corresponding to positions 429-431 of AAV2 (SEQ ID NO: 1), resulting in a leucine to phenylalanine substitution at amino acid position 37 of the Rep 68 protein. Correction of the helper plasmid sequence to restore the leucine-encoding wild type AAV2 codon resulted in an increased vg/ml yield and an approximately two-fold increase in vector genomes to total particles ratio (i.e. % full particles), when directly compared against the ‘mutated’ helper plasmid. The corrected helper plasmid sequence was used in all subsequent work, including production of the rAAV batches analysed in Example 3.

### Example 3—Determination of Replication Competent AAV (rcAAV) Frequency in rAAV Produced by Trans-Split Two-Plasmid System

rcAAV Testing

rAAV purified bulk drug substance, manufactured in large-scale using the iCellis® bioreactor with cells transfected with the two-plasmid system and purified using a number of downstream processes to remove product impurities, were subjected to a limit test for the presence of rcAAV. The rAAV batches were produced using vector plasmid comprising the engineered cap gene and (i) α-galactosidase A expression cassette (as mentioned in Example 1) or (ii) a Factor IX expression cassette identical to that mentioned in Example 1 except for a different partially codon-optimised Factor IX coding sequence. The two plasmid system used to produce these rAAV batches used the shortened vector plasmid backbone and corrected helper plasmid sequence as mentioned in Examples 1 and 2, respectively. In the limit test, HEK293 cells are transduced by the rAAV at its most concentrated form (drug substance post-purification and pre-formulation) in the presence or absence of wild-type adenovirus. Three successive rounds of virus amplification are conducted as described below.

Cells are seeded into T75/T175 flasks. At 80%+/−10 confluency and >90% viability, cells are transduced with and without helper wild-type Adenovirus serotype 5 (“Ad5 wt”) at a multiplicity of infection (MOI) of 7.8×10³ and the rAAV product to be tested. Following 2 days incubation, the cells are harvested by detaching mechanically. Three sets of cells are collected and centrifuged from round one: 1) retained for back up (stored in case repeat analysis required); 2) for DNA purification and analysis; 3) for a second round of transduction. Cells for purification and following rounds of transduction are lysed by freeze/thaw (3×) in dry ice/ethanol to water bath 37° C., and Ad5 wt inactivated by incubation at 56° C. degrees for 30 mins and centrifugation at 1500 g for 2 mins. The cells seeded for second transduction are transduced with lysate from first harvest, with and without Ad5 wt, and the entire process is repeated to collect samples from harvest 2. The cells seeded for the third transduction are transduced with lysate from second harvest, with and without Ad5 wt and the entire process repeated, to collect samples from harvest 3.

Genomic DNA is extracted from each of the three amplification steps using DNeasy® Tissue kit (Qiagen) and quantified by UV at A260/A280. The presence of rcAAV is detected by real-time quantitative PCR (qPCR): DNA is isolated from the HEK293 cells and two sequences are amplified from the isolated DNA using 1) a primer specific for the rep-encoding sequence of AAV2 (“rep”), and 2) an endogenous housekeeping gene in HEK293 cells (human albumin; hAlb).

DNA at a concentration of 100 ng is added to the 25 μl qPCR reaction. The copy number of rep is calculated relative to the plasmid standard range (from 100 to 1×10⁸ copies per qPCR reaction) and relative copy numbers of the rep sequence per cell calculated as the ratio of the rep copies and the human albumin sequence copies, multiplied by two (hAlb, two copies/cell). Increased levels of the rep sequence per cell in consecutive amplification rounds indicates the presence of rcAAV.

The positive control is wild-type AAV2 with Adenovirus (Ad5 wt); they are tested alone or in the presence of the rAAV product sample as inhibition controls. The control confirms the detection limit for the test (LOD) being between 10 rcAAV 1×10¹⁰ to 10 rcAAV per 1×10¹¹ vg of rAAV product sample for AAV2. The LOD is an assay and product-dependent parameter. The negative controls consist of non-infected cells (with or without adenovirus) and cells transduced with wt AAV2 (without adenovirus). Replication is established when the rep sequence copy number per cell is >10 in at least 1 of the 3 amplification rounds, and in subsequent rounds if more than one. Absence of rep is reported as ‘no replication’, meaning <10 rcAAV in 1×10¹⁰ to 1×10¹¹ vg of test sample. Detection of rep sequence is reported as ‘replication’, i.e. detection of rcAAV in 1×10¹⁰ to 1×10¹¹ vg of test sample.

Results

The limit test was performed on several batches of each of the α-galactosidase A- and Factor IX-containing rAAV products. Replication competent positive controls of wild-type AAV2 coinfection with Ad5 wt confirmed rcAAV detection by the assay. The rcAAV (AAV2) positive controls spiked into the rAAV product sample demonstrated no sample inhibition. The positive controls confirmed that the detection limit for the test (LOD) was 10 rcAAV per 1×10¹¹ vg of Factor IX-containing rAAV product sample, and 10 rcAAV per 1×10¹⁰ or 1×10¹¹ vg of α-galactosidase A-containing rAAV product sample. The negative controls of non-infected cells (with or without adenovirus) and cells transduced with wt AAV2 (without adenovirus) were negative for rcAAV.

Thus, the tested rAAV product batches produced using the two-plasmid system were shown to contain <1 rcAAV per 1×10¹⁰ vg of Factor IX-containing rAAV product, and <1 rcAAV per 1×10⁹-1×10¹⁰ vg of α-galactosidase A-containing rAAV product.

### Example 4—Two-Plasmid System: Comparison of Helper:Vector Plasmid Ratios in Context of Additional Transgenes

Cell Cultivation

HEK293 cells were maintained in adherent culture under standard conditions at 37° C., 95% relative humidity, and 5% v/v CO₂ in Dulbecco's Modified Eagle's Medium (DMEM) supplemented with 5% Fetal Bovine Serum (FBS) and 2 mM L-glutamine. Cellular confluence during passaging ranged from 40-95%.

Transfection of HEK293 Cells and Preparation of Cell Lysates

HEK293 cells were transfected with helper plasmid and vector plasmid (the latter comprising the engineered cap gene and beta-Glucocerebrosidase [GBA] or Factor VIII [FVIII] expression cassettes) using different molar plasmid ratios (from helper:vector 1:0.75 to 1:4.5) while maintaining the total plasmid DNA amount. 1.5×10¹ viable cells per cm² culture area were seeded in 6-cm dishes in a volume of 3 ml DMEM, 5% FBS, 2 mM L-glutamine the day before transfection resulting in 60-70% confluency on the day of transfection. PEI-DNA complexes were prepared in DMEM without supplements using the linear polyethylenimine transfection reagent PEIpro™ (Polyplus) according to the manufacturer's manual. An amount of 6 μg total plasmid DNA and a PEI-to-DNA ratio of 2:1 was maintained independent of the applied plasmid combination ratios. Cells were cultured until day 3 post-transfection, harvested in the medium and lysed by three freeze-thaw cycles (−80° C. and 37° C.). Cell debris was removed by centrifugation at 10,000×g for 5 min.

Quantification of rAAV Vector Genomes

The AAV vector genome assay is based on a quantitative polymerase chain reaction (qPCR) specific for the promoter sequence of the GBA expression cassette or specific for the coding sequence of the FVIII expression cassette.

Cell lysate test samples were subjected to a nuclease treatment procedure in order to remove non-packed vector genomes prior to performing the qPCR. To that aim the samples were pre-diluted 1:250 in nuclease-free water containing 0.125% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, suitable trending controls containing unpurified cell lysates with known AAV vector genome titres and spike-in controls using plasmid DNA carrying the promoter sequence (in case of the GBA expression cassette) or FVIII coding sequence (in case of the FVIII expression cassette) were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with following program steps: 95° C. 5 min; 39 cycles (95° C. 10 s, 60° C. 30 s, plate read); 95° C. 10 sec; 60-95° C. (+0.5° C./step), 10 sec; plate read. To control for the quality of the qPCR, a trending control with known AAV vector genome titre was measured in parallel. To check for contaminations, a no template control (NTC, 5 μl H₂O) was also included. Standard row, test samples and controls were measured in triplicates for each dilution. Purified virus test samples and trending control were generally measured in 3 different dilutions in EB buffer (10 mM Tris-Cl, pH 8.5). Turbo DNase treated cell lysate test samples were directly used in the qPCR without any further dilution. Data were analysed using the CFX Manager™ Software 3.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the promoter (for the GBA expression cassette) or coding sequence (for the FVIII expression cassette) genetic material, in the form of linearised plasmids, were serially diluted to create standard curves and sample vector genome titres were interpolated from the respective standard curves.

Quantification of rAAV Particles (Capsids)

The AAV2 Titration ELISA method is a measure of total AAV particles (capsids) and is based on a commercially available kit (Progen™, Heidelberg, Germany; catalogue number PRATV). This sandwich immunometric technique utilises monoclonal antibody A20 (Wobus et al (2000), J Virol, 74:9281-9293) for both capture and detection. The antibody is specific for a conformational epitope present on assembled capsids of serotypes AAV2, AAV3, and the engineered capsid used in these experiments.

The AAV2 Titration ELISA kit was used to quantify total AAV particles in cell lysates and purified virus preparations according to the manufacturer's instructions. In brief, 100 μl diluted AAV2 Kit Control, test samples, or trending control of engineered capsid with known total particle titre were added per well of a microtiter plate coated with monoclonal antibody A20 and incubated for 1 h at 37° C. Standard row, test samples and controls were measured in duplicates for each dilution. In a second step, 100 μl of pre-diluted biotin-conjugated monoclonal antibody A20 (1:20 in Assay Buffer [ASSB]) were added and incubated for 1 h at 37° C. Then, 100 μl of a pre-diluted streptavidin peroxidase conjugate (1:20 in ASSB) were added and incubated for 1 h at 37° C. 100 μl substrate solution (TMB [Tetramethylbenzidine]) were added and after incubation for 15 min, the reaction was stopped using 100 μl stop solution. The absorbance was measured photochemically at 450 nm using the SpectraMax M3 microplate reader (Molecular Devices, San Jose, USA). Data was analysed with the SoftMax Pro 7.0 Software (Molecular Devices).

The test samples were diluted into the assay range and AAV total particle concentrations were determined by interpolation using the standard curve which was prepared using the provided AAV2 Kit Control. ASSB was used as blank.

Vector Genome to Total Particle Ratio

The ratio of vector genomes to total AAV particles is expressed as a percentage. This is based on the vector genome titre (determined by qPCR, as described above) and the number of total AAV particles (determined by the capsid ELISA, as described above).

Results

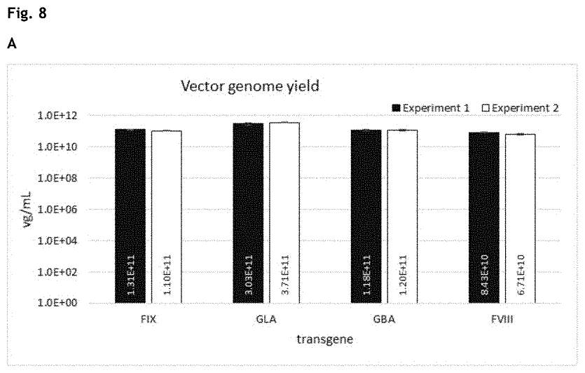

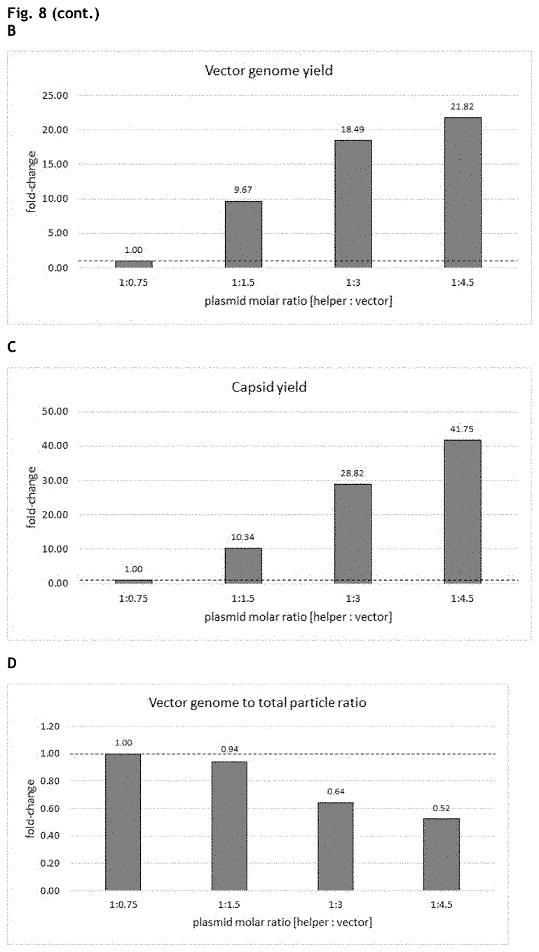

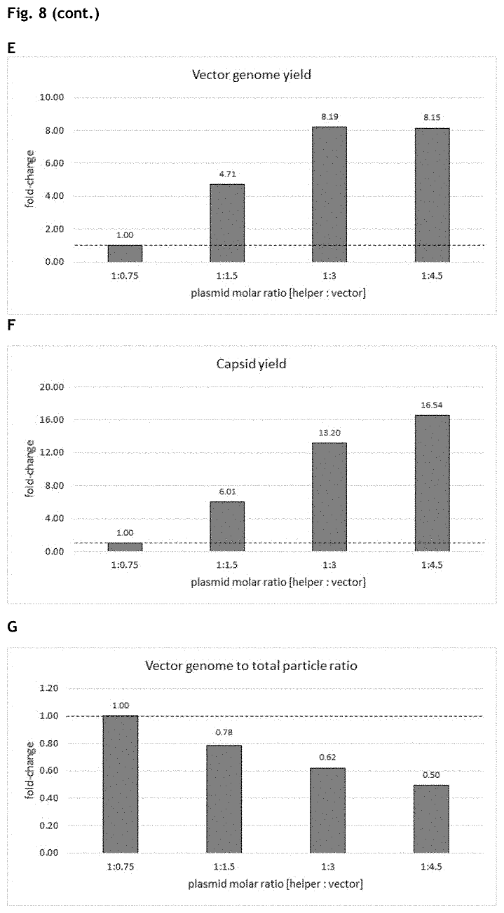

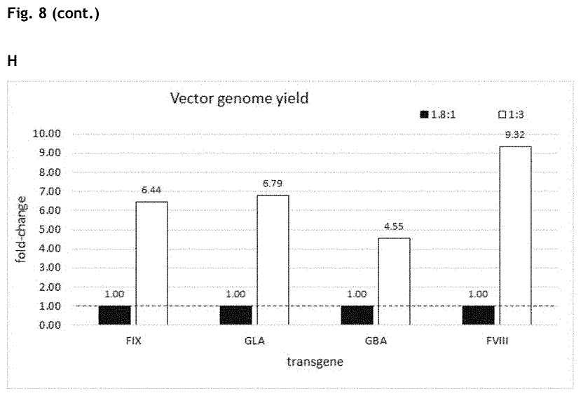

The comparison of different helper:vector plasmid ratios in context of GBA (FIGS. 8B-D) and FVIII (FIGS. 8E-G) transgenes confirmed the observation in Example 2 that elevation of the proportion of vector plasmid led to both higher particle and vector genome yields. As the relative increase in particle (capsid) yields was again more pronounced than the relative increase in vector genome yields a gradual decrease in the vector genome to total particles ratio was noticed (FIGS. 8D and 8G). A plasmid ratio of helpervector 1:3 resulted in an almost maximal (GBA) or maximal (FVIII) increase in vector genome titre whilst maintaining the balance between high yields and acceptable vector genome to total particle ratios.

### Example 5—Two-Plasmid System: Evaluation in Context of Various Transgenes

Cell Cultivation

HEK293T cells were maintained in adherent culture under standard conditions at 37° C., 95% relative humidity, and 5% v/v CO₂ in Dulbecco's Modified Eagle's Medium (DMEM) supplemented with 10% Fetal Bovine Serum (FBS) and 1% GlutaMax™ (L-alanine-L-glutamine dipeptide). Cellular confluence during passaging ranged from 40-95%.

HEK293 cells were maintained in adherent culture under standard conditions at 37° C., 95% relative humidity, and 5% v/v CO₂ in Dulbecco's Modified Eagle's Medium (DMEM) supplemented with 5% Fetal Bovine Serum (FBS) and 2 mM L-glutamine. Cellular confluence during passaging ranged from 40-95%.

Transfection of HEK293T or HEK293 Cells and Preparation of Cell Lysates

HEK293T or HEK293 cells were transfected with helper plasmid and vector plasmid (the latter comprising the engineered cap gene and Factor IX, alpha-Galactosidase A [GLA], beta-Glucocerebrosidase [GBA] or Factor VIII expression cassettes) using molar plasmid ratio 1:3 (helper:vector) while maintaining the total plasmid DNA amount. 1.5×10⁵ viable cells per cm² culture area were seeded in 6-cm dishes in a volume of 3 ml DMEM, 10% FBS, 1% GlutaMax™ (HEK293T) or in a volume of 3 ml DMEM, 5% FBS, 2 mM L-glutamine (HEK293) the day before transfection resulting in 60-70% confluency on the day of transfection. PEI-DNA complexes were prepared in DMEM without supplements using the linear polyethylenimine transfection reagent PEIpro™ (Polyplus) according to the manufacturer's manual. An amount of 6 μg total plasmid DNA and a PEI-to-DNA ratio of 2:1 was maintained independent of the applied plasmid combinations. Cells were cultured until day 3 post-transfection, harvested in the medium and lysed by three freeze-thaw cycles (−80° C. and 37° C.). Cell debris was removed by centrifugation at 10,000×g for 5 min. AAV vectors containing the Factor IX or GLA expression cassette were manufactured in HEK293T cells whereas AAV vectors containing the GBA or Factor VIII expression cassette were manufactured in HEK293 cells.

Quantification of rAAV Vector Genomes

The AAV vector genome assay is based on a quantitative polymerase chain reaction (qPCR) specific for the promoter sequence of the rAAV expression cassette.

Cell lysate test samples were subjected to a nuclease treatment procedure in order to remove non-packed vector genomes prior to performing the qPCR. To that aim the samples were pre-diluted 1:250 in nuclease-free water containing 0.125% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, a trending control containing unpurified cell lysate with a known AAV vector genome titre and spike-in controls using plasmid DNA carrying the promoter sequence were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with following program steps: 95° C. 5 min; 39 cycles (95° C. 10 s, 60° C. 30 s, plate read); 95° C. 10 sec; 60-95° C. (+0.5° C./step), 10 sec; plate read. To control for the quality of the qPCR, a trending control with known AAV vector genome titre was measured in parallel. To check for contaminations, a no template control (NTC, 5 μl H₂O) was also included. Standard row, test samples and controls were measured in triplicates for each dilution. Purified virus test samples and trending control were generally measured in 3 different dilutions in EB buffer (10 mM Tris-Cl, pH 8.5). Turbo DNase treated cell lysate test samples were directly used in the qPCR without any further dilution. Data were analysed using the CFX Manager™ Software 3.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the promoter genetic material, in the form of a linearised plasmid, were serially diluted to create a standard curve and sample vector genome titre was interpolated from the standard curve.

Results

Four different vector genomes containing transgenes and expression cassettes of distinct sizes (Factor IX [FIX], alpha-Galactosidase A [GLA], beta-Glucocerebrosidase [GBA] and Factor VIII [FVIII]) were packaged into the engineered capsid in two independent experiments each. As shown in FIG. 8A robustly high vector genome yields were achieved independent of the expression cassette and both when HEK293T and HEK293 cells were used for vector production. These results demonstrate that the plasmid system can be used as a platform for the manufacturing of various transgene encoding AAV vectors in different human embryonic kidney 293 cell lineages.

### Example 6—Two-Plasmid System: Evaluation in Context of Various Serotype and Engineered Cap Genes

Cell Cultivation

HEK293T cells were maintained in adherent culture under standard conditions at 37° C., 95% relative humidity, and 5% v/v CO₂ in Dulbecco's Modified Eagle's Medium (DMEM) supplemented with 10% Fetal Bovine Serum (FBS) and 1% GlutaMax™ (L-alanine-L-glutamine dipeptide). Cellular confluence during passaging ranged from 40-95%.

Transfection of HEK293T Cells and Preparation of Cell Lysates

HEK293T cells were transfected with helper plasmid and vector plasmid (the latter comprising the Factor IX expression cassette and AAV-2, AAV-5, AAV-8, AAV-9 or the engineered cap gene) using molar plasmid ratio 1:3 (helper:vector) while maintaining the total plasmid DNA amount. 1.5×10⁵ viable cells per cm² culture area were seeded in 6-cm dishes in a volume of 3 ml DMEM, 10% FBS, 1% GlutaMax™ the day before transfection resulting in 60-70% confluency on the day of transfection. PEI-DNA complexes were prepared in DMEM without supplements using the linear polyethylenimine transfection reagent PEIpro™ (Polyplus) according to the manufacturer's manual. An amount of 6 μg total plasmid DNA and a PEI-to-DNA ratio of 2:1 was maintained independent of the applied plasmid combinations. Cells were cultured until day 3 post-transfection, harvested in the medium and lysed by three freeze-thaw cycles (−80° C. and 37° C.). Cell debris was removed by centrifugation at 10,000×g for 5 min.

Quantification of rAAV Vector Genomes

The AAV vector genome assay is based on a quantitative polymerase chain reaction (qPCR) specific for the promoter sequence of the rAAV expression cassette.

Cell lysate test samples were subjected to a nuclease treatment procedure in order to remove non-packed vector genomes prior to performing the qPCR. To that aim the samples were pre-diluted 1:250 in nuclease-free water containing 0.125% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, a trending control containing unpurified cell lysate with a known AAV vector genome titre and spike-in controls using plasmid DNA carrying the promoter sequence were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with following program steps: 95° C. 5 min; 39 cycles (95° C. 10 s, 60° C. 30 s, plate read); 95° C. 10 sec; 60-95° C. (+0.5° C./step), 10 sec; plate read. To control for the quality of the qPCR, a trending control with known AAV vector genome titre was measured in parallel. To check for contaminations, a no template control (NTC, 5 μl H₂O) was also included. Standard row, test samples and controls were measured in triplicates for each dilution. Purified virus test samples and trending control were generally measured in 3 different dilutions in EB buffer (10 mM Tris-Cl, pH 8.5). Turbo DNase treated cell lysate test samples were directly used in the qPCR without any further dilution. Data were analysed using the CFX Manager™ Software 3.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the promoter genetic material, in the form of a linearised plasmid, were serially diluted to create a standard curve and sample vector genome titre was interpolated from the standard curve.

Results

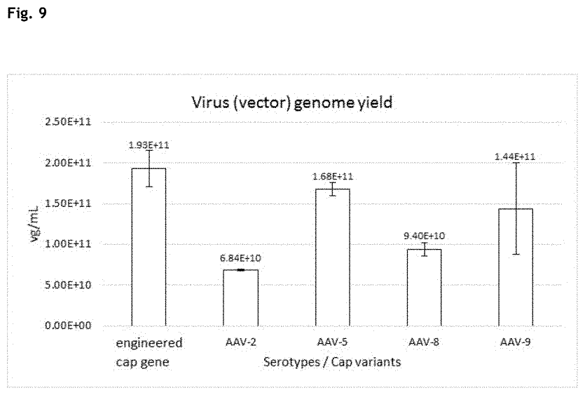

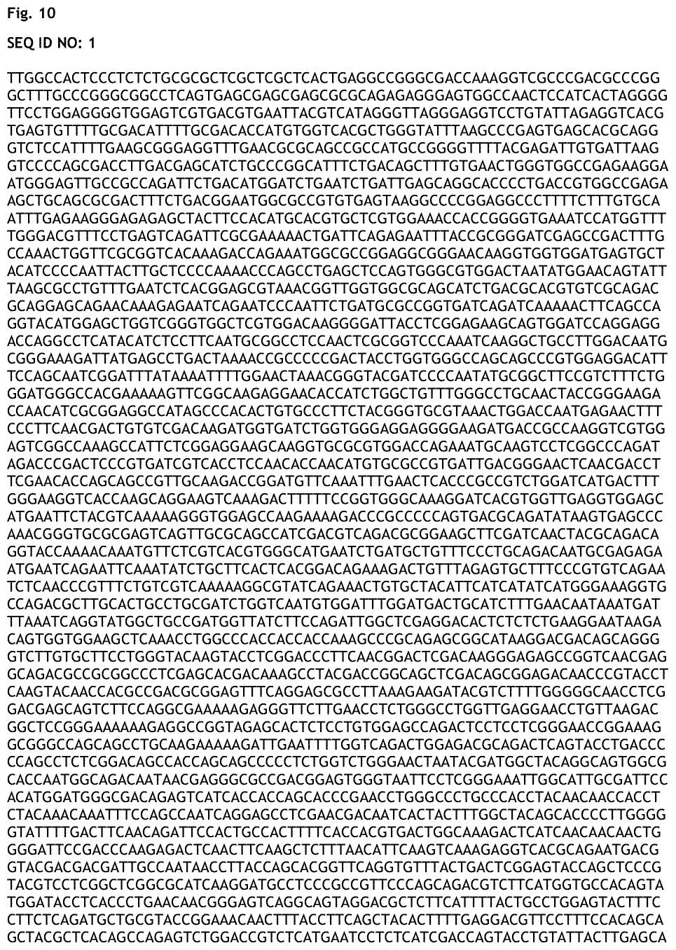

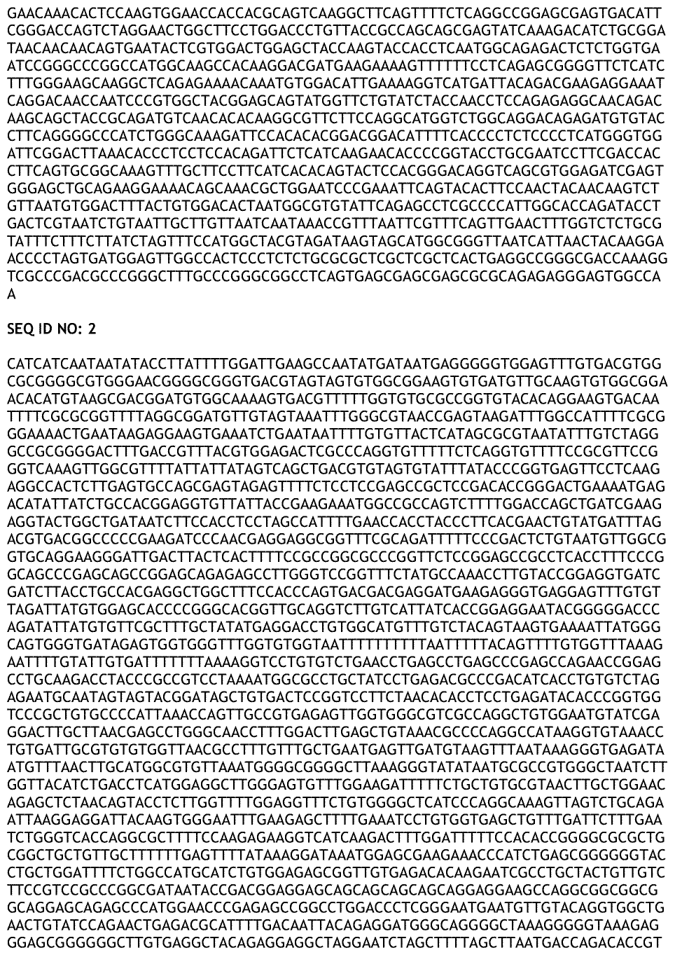

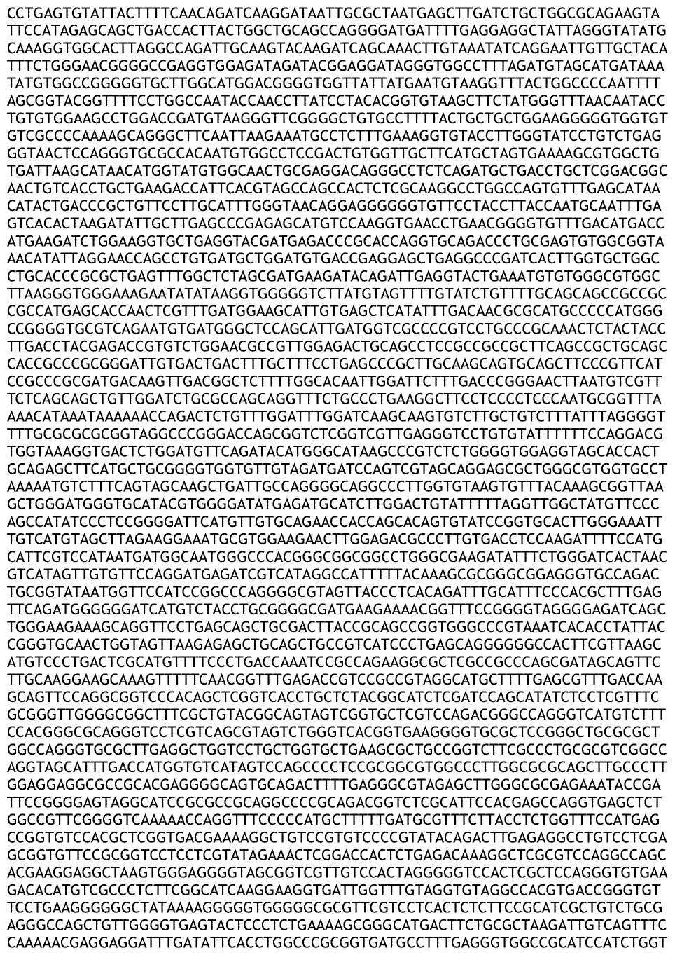

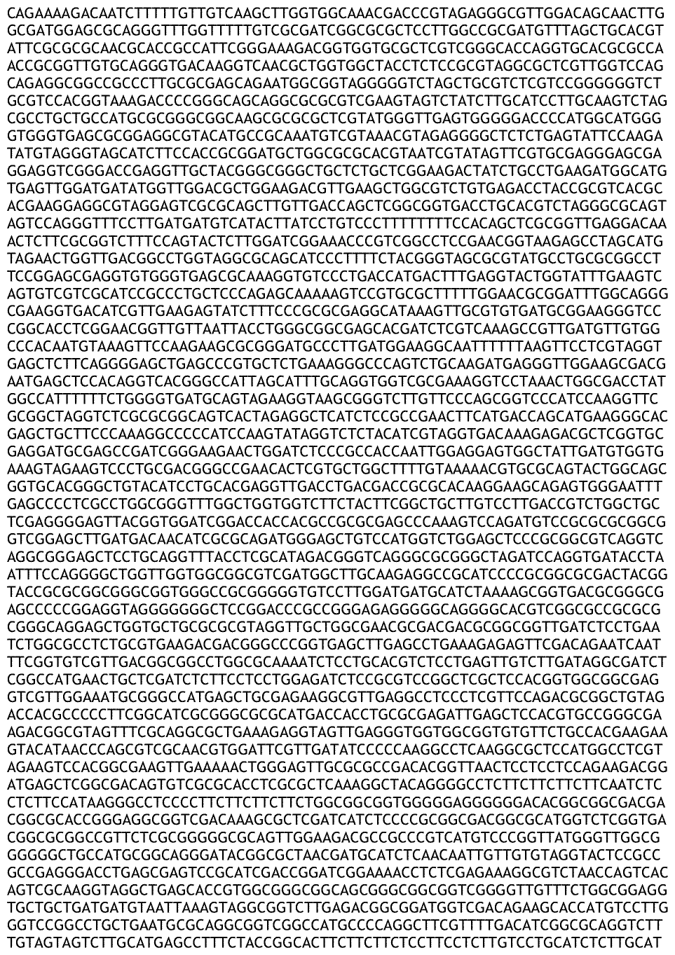

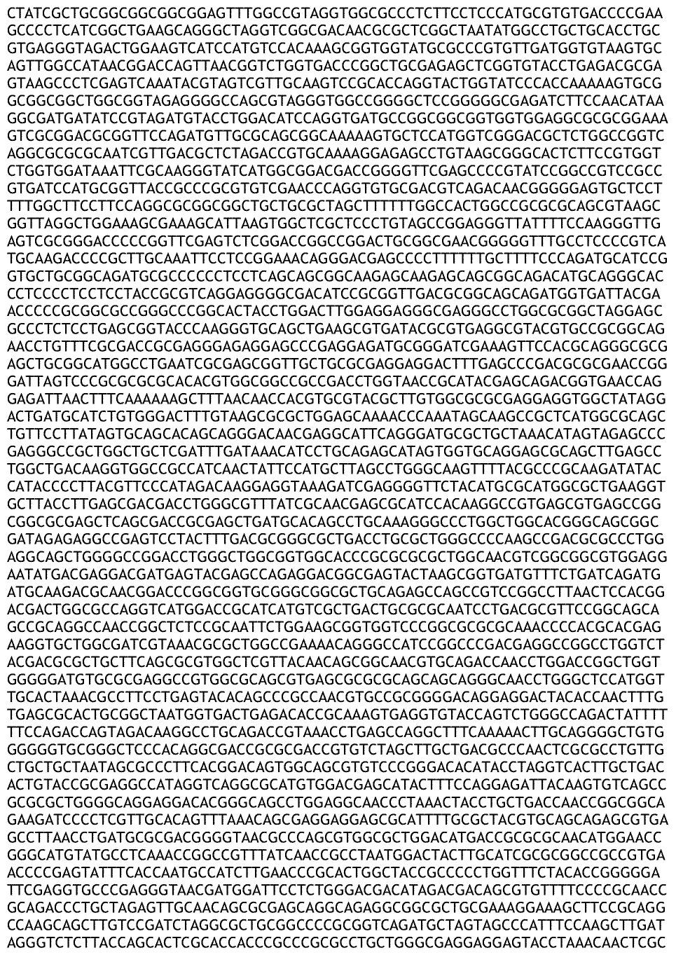

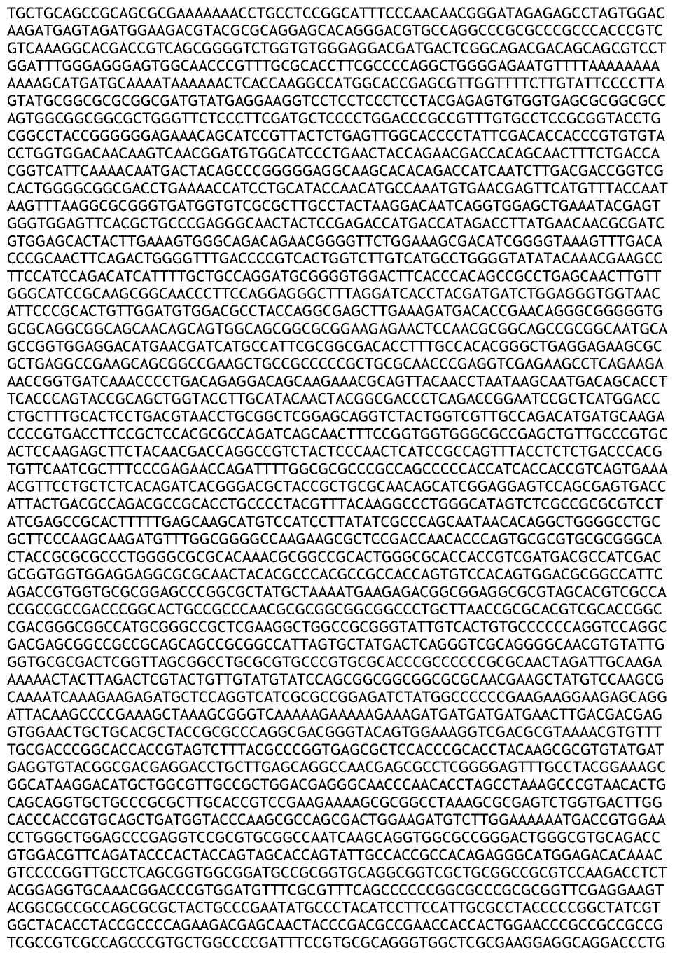

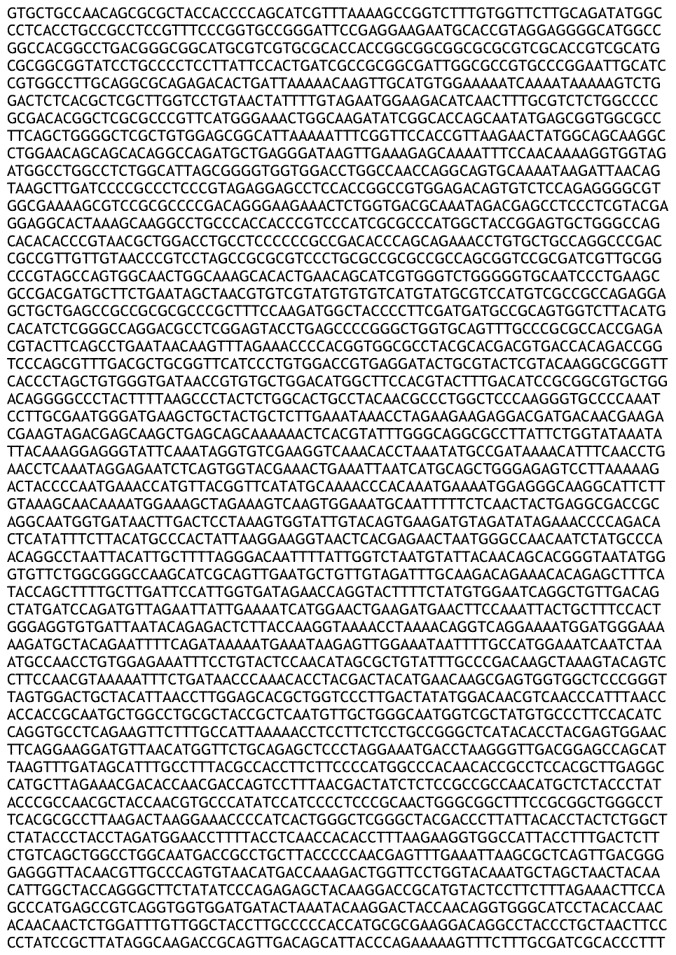

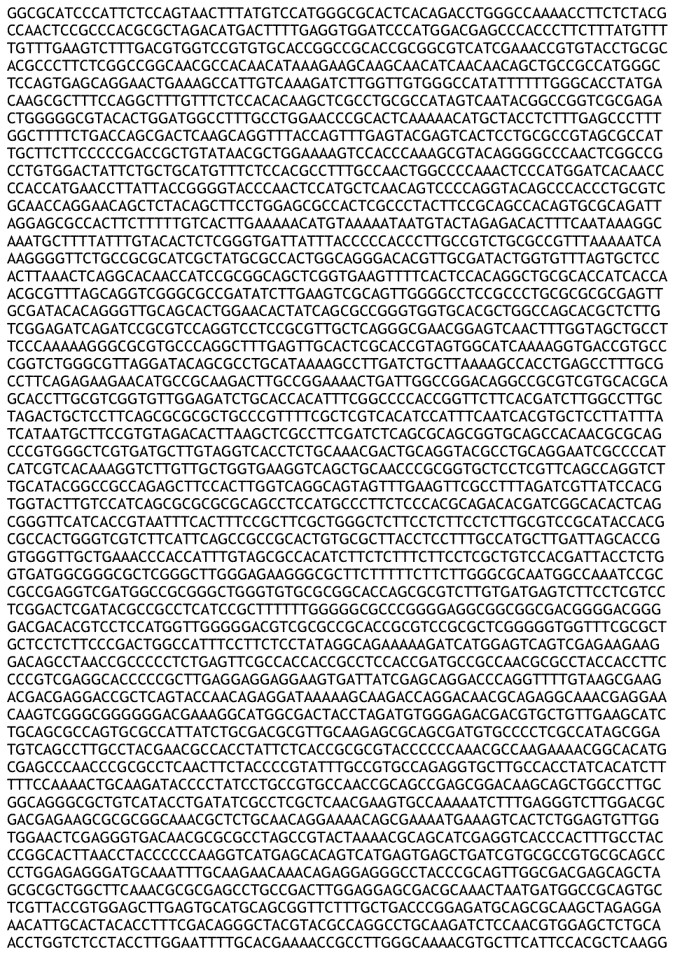

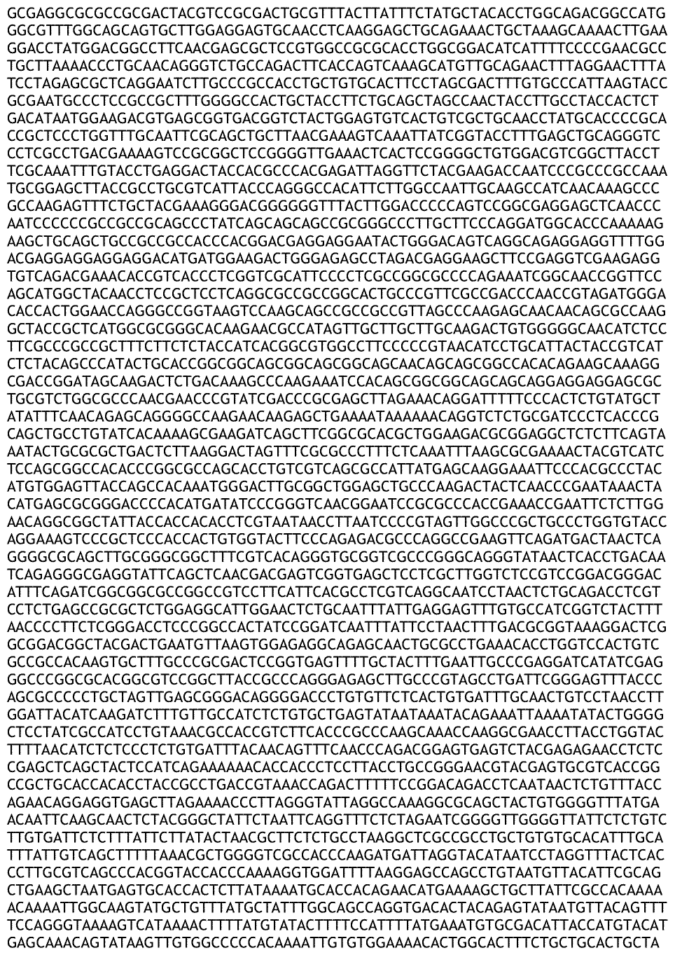

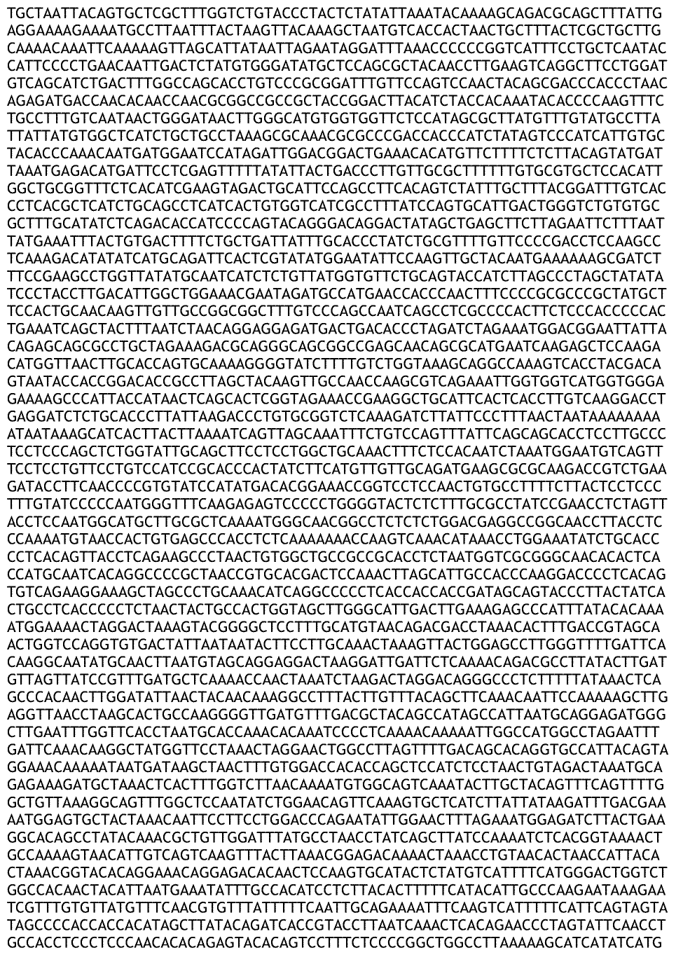

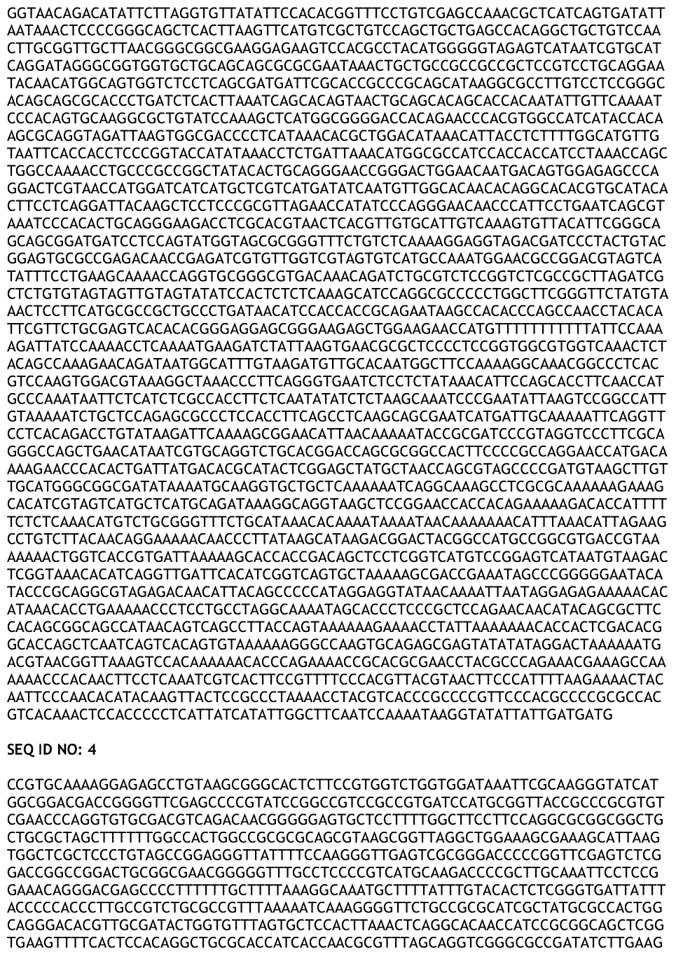

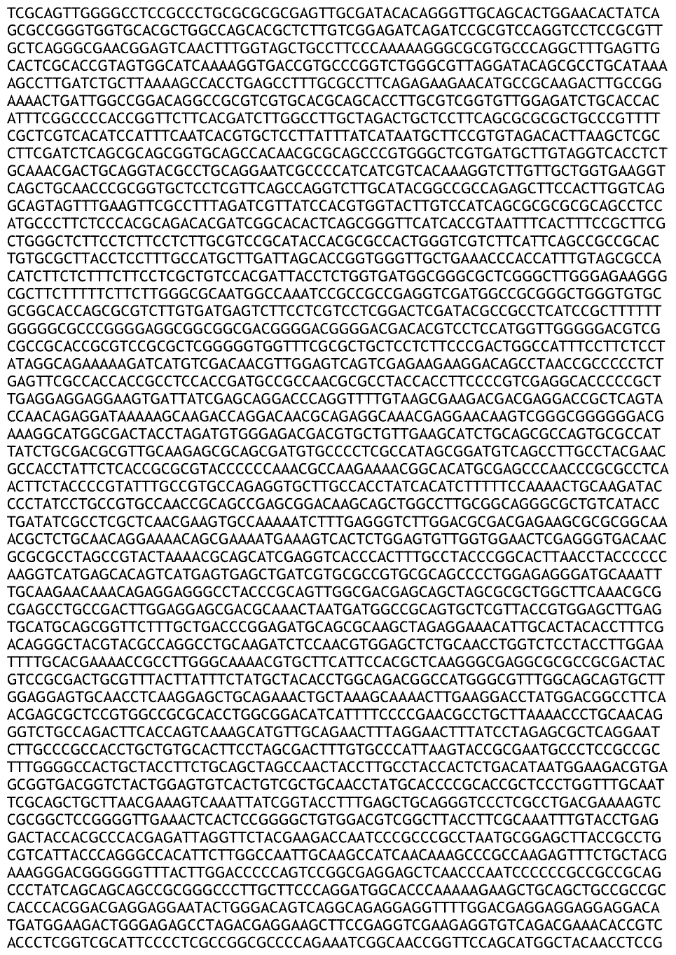

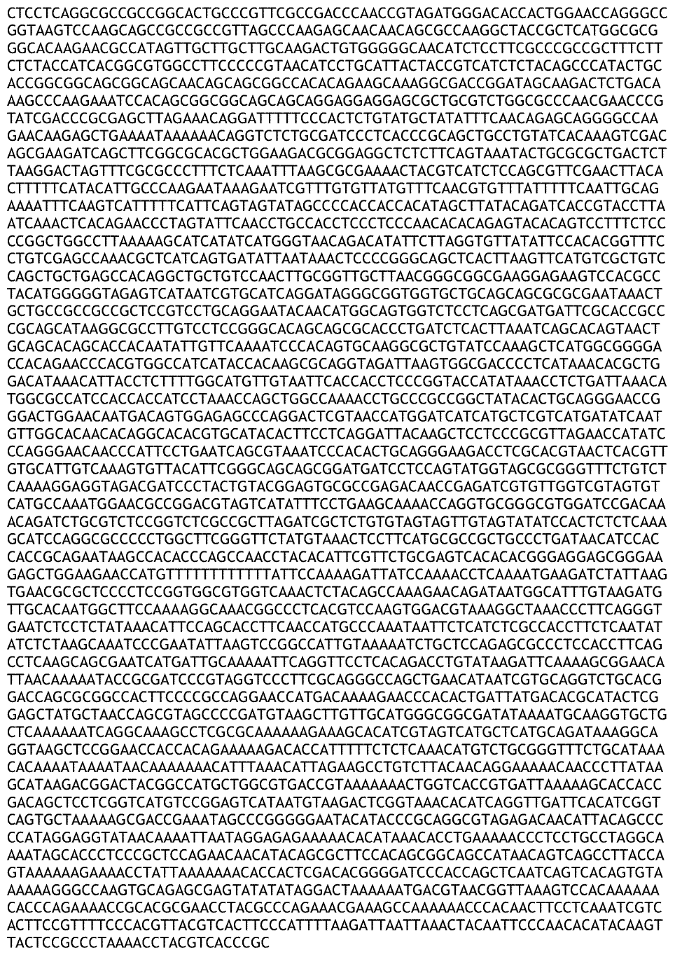

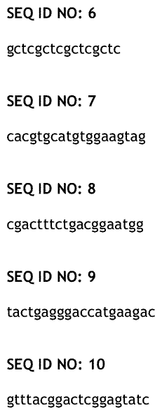

Factor IX encoding vector genomes were packaged into the engineered capsid as well as into the naturally occurring AAV-2, AAV-5, AAV-8 and AAV-9 serotype capsids. The obtained vector genome yields depicted in FIG. 9 were within a reasonable range of less than a 3-fold difference between the highest and lowest value. Therefore, the plasmid system is considered a suitable platform for the production of AAV vectors in context of various capsid variants.

### Example 7—Two-Plasmid System: Comparison of Selected Helper:Vector Plasmid Ratios in Context of Various Transgenes

Cell Cultivation

HEK293T cells were maintained in adherent culture under standard conditions at 37° C., 95% relative humidity, and 5% v/v C02 in Dulbecco's Modified Eagle's Medium (DMEM) supplemented with 10% Fetal Bovine Serum (FBS) and 1% GlutaMax™ (L-alanine-L-glutamine dipeptide). Cellular confluence during passaging ranged from 40-95%.

Transfection of HEK293T Cells and Preparation of Cell Lysates

HEK293T cells were transfected with helper plasmid and vector plasmid (the latter comprising the engineered cap gene and Factor IX [FIX], alpha-Galactosidase A [GLA], beta-Glucocerebrosidase [GBA] or Factor VIII [FVIII] expression cassettes) using two different molar plasmid ratios (helpervector 1.8:1 and 1:3) while maintaining the total plasmid DNA amount. 1.5×10⁵ viable cells per cm² culture area were seeded in 6-cm dishes in a volume of 3 ml DMEM, 10% FBS, 1% GlutaMax™ the day before transfection resulting in 60-70% confluency on the day of transfection. PEI-DNA complexes were prepared in DMEM without supplements using the linear polyethylenimine transfection reagent PEIpro™ (Polyplus) according to the manufacturer's manual. An amount of 6 μg total plasmid DNA and a PEI-to-DNA ratio of 2:1 was maintained independent of the applied plasmid combination ratios. Cells were cultured until day 3 post-transfection, harvested in the medium and lysed by three freeze-thaw cycles (−80° C. and 37° C.). Cell debris was removed by centrifugation at 10,000×g for 5 min.

Quantification of rAAV Vector Genomes

The AAV vector genome assay is based on a quantitative polymerase chain reaction (qPCR) specific for the promoter sequence of the rAAV expression cassettes.

Cell lysate test samples were subjected to a nuclease treatment procedure in order to remove non-packed vector genomes prior to performing the qPCR. To that aim the samples were pre-diluted 1:250 in nuclease-free water containing 0.125% Pluronic F-68. 25 μl of the pre-dilution were used for the digest with 2 units of Turbo DNase (ThermoFisher Scientific, Waltham, USA) and 1× Turbo DNase reaction buffer, resulting in a total reaction volume of 29 μl. Incubation was performed for 1 h at 37° C. Afterwards, 1 volume of 0.4 M NaOH was added and the samples were incubated for 45 min at 65° C. 1035 μl nuclease-free water supplemented with 0.1% Pluronic F-68 were added together with 30 μl 0.4 M HCl. To control for the quality of the Turbo DNase digest, a trending control containing unpurified cell lysate with a known AAV vector genome titre and spike-in controls using plasmid DNA carrying the promoter sequence were measured in parallel.

Per sample, 12.5 μl QuantiFast SYBR Green PCR Master Mix (Qiagen, Venlo, Netherlands) were mixed with 0.75 μl of qPCR primer working stock solution (containing 10 μM of each primer) and filled up to a volume of 20 μl with nuclease-free water. 5 μl of Turbo DNase treated cell lysate or purified virus test sample were added to the mix (total reaction volume 25 μl, final primer concentration in the reaction 300 nM each) and qPCR was performed in a CFX 96 Touch Real Time PCR cycler (Bio-Rad Laboratories Inc., Hercules, USA) with following program steps: 95° C. 5 min; 39 cycles (95° C. 10 s, 60° C. 30 s, plate read); 95° C. 10 sec; 60-95° C. (+0.5° C./step), 10 sec; plate read. To control for the quality of the qPCR, a trending control with known AAV vector genome titre was measured in parallel. To check for contaminations, a no template control (NTC, 5 μl H₂O) was also included. Standard row, test samples and controls were measured in triplicates for each dilution. Purified virus test samples and trending control were generally measured in 3 different dilutions in EB buffer (10 mM Tris-Cl, pH 8.5). Turbo DNase treated cell lysate test samples were directly used in the qPCR without any further dilution. Data were analysed using the CFX Manager™ Software 3.1 (Bio-Rad Laboratories Inc.).

Melting curve analysis confirmed the presence of only one amplicon. Amplification results in nascent double stranded DNA amplicons detected with the fluorescent intercalator SYBR Green to monitor the PCR reaction in real time. Known quantities of the promoter genetic material, in the form of a linearised plasmid, were serially diluted to create a standard curve and sample vector genome titre was interpolated from the standard curve.

Results

The four different vector plasmids as already used in Example 5 and containing transgenes of distinct sizes were transfected in the previously applied helper:vector plasmid ratio of 1.8:1 and in the plasmid ratio of 1:3 into HEK293T cells. In all instances substantially elevated vector genome yields were observed for the 1:3 plasmid ratio ranging from 4.55-fold to 9.32-fold increases (FIG. 8H) underlining the advantage of the 1:3 plasmid ratio in the context of AAV vectors containing various expression cassettes.

### Example 8—Investigating Replication Competent AAV (rcAAV, which Includes Rep-Deficient RcAAV Cap-Deficient rcAAV and Pseudo-Wild Type rcAAV) Produced by the Trans-Split Two-Plasmid System

The limit test from Example 3 showed that the rAAV product batches produced using the trans-split two-plasmid system contained <1 rcAAV per 1×10¹⁰ vg of Factor IX-containing rAAV product, and <1 rcAAV per 1×10⁹-1×10¹⁰ vg of α-galactosidase A-containing rAAV product. Thus producing any rcAAV is a rare event. To investigate whether any rcAAV is being produced by the trans-split two-plasmid system and how such rcAAV may compare to any rcAAV produced by a conventional “non-split” configuration, the following series of experiments were performed.

Plasmids

For the trans-split two-plasmid system, the helper and vector plasmids were constructed as set out in Example 1. FIGS. 2 and 3 provide schematics of the helper and vector plasmids, respectively. The shortened vector plasmid backbone and corrected helper plasmid sequence were used as mentioned in Examples 1 and 2, respectively. The vector plasmid comprised the engineered cap gene and Factor IX expression cassette identical to that mentioned in Example 3.

Two plasmids in a conventional “non-split” configuration were used for comparison. One plasmid contained the same AdV helper and vector genome (Factor IX expression cassette) sequences as used in the trans-split two-plasmid system. The other plasmid (also referred to herein as “P-143”) contained the same cap gene sequence as used in the trans-split two-plasmid system and an AAV2 rep cassette containing all four rep genes such that Rep and Cap functions are not split between the two plasmids (FIG. 14). Thus, the cap and rep genes are in the wild-type configuration as shown in FIG. 1.

The helper plasmid used is also referred to as “P-150” and the vector plasmid used is also referred to as “P-160”.

Cell Cultivation

HEK293T cells were cultivated as described in Example 2 under the section “Cell cultivation”.

Transfection of HEK293T Cells and Purifying the rAAV

HEK293T cells were transfected with helper plasmid and vector plasmid using different molar plasmid ratios for the trans-split two-plasmid system and for non-split system, while maintaining the total plasmid DNA amount. The helper:vector ratio for the trans-split two-plasmid system was 1:3 and the AdV helper-expression cassette:cap-rep ratio for non-split system was 1.8:1.8×10⁴ viable cells per cm² culture area were seeded in 15-cm dishes in a volume of 25 ml DMEM, 10% FBS, 1% GlutaMax™ the day before transfection resulting in 60-70% confluency on the day of transfection. PEI-DNA complexes were prepared in DMEM without supplements using the linear polyethylenimine transfection reagent PEIpro™ (Polyplus) according to the manufacturer's manual. An amount of 42 μg total plasmid DNA per plate and a PEI-to-DNA ratio of 2:1 was maintained independent of the applied plasmid combination ratios. Cells were cultured until day 3 post-transfection, harvested in the medium and lysed by three freeze-thaw cycles (−80° C. and 37° C.). A pool of seven dishes were treated with Denarase (Sartorius Stedim Biotech) to remove residual plasmid DNA and purified by affinity chromatography. qPCR was used to quantify the rAAV vector genomes (i.e. determine the rAAV vector genome titres) for purified trans-split two-plasmid system and non-split system rAAV finally formulated in PBS. The qPCR was performed by amplifying a region in the promoter sequence of the rAAV expression cassette. The qPCR was performed as set out above in Example 2 under the section “Quantification of rAAV vector genomes”. A titre of 1.3×10¹² vg/ml was obtained for the non-split system preparation and a titre of 8.2×10¹² vg/ml was obtained for the trans-split two-plasmid system preparation.

Enrichment of rcAAV (which Includes Rep-Deficient rcAAV, Cap-Deficient rcAAV and Pseudo-Wild Type rcAAV) and Isolation of Vector DNA

As any rcAAV being generated from the trans-split two-plasmid system is a rare event, enrichment for the rcAAV species was performed before the further Southern blot, qPCR and PCR analysis was performed.

In order to enrich for rcAAV particles, HEK293T cells were infected with rAAV generated from the trans-split two-plasmid system (also referred to as “split sample”) and the non-split system (also referred to as “non-split sample”). HEK293T cells were infected with the rAAV samples in their most concentrated form and co-infected with Ad5 wt. In total, two successive rounds of infection were performed as described below. For AAV particles to be produced during these two rounds, a HEK293T cell would need to be infected with an rcAAV which comprises both Rep and Cap functions, or infected with more than one rcAAV where the combination of rcAAVs present in the cell provide Rep and Cap functions.

Cells were seeded in 96-well plates with 1.2e5 vc/cm² (vc=viable cells). The next day, at 80%+/−10% confluency, cells were infected with 25 μl of the undiluted rAAV sample and co-infected with Ad5 wt at a MOI of 2.5 in a total volume of 150 μl per well. To act as a negative control, certain wells were only infected with Ad5 wt at an MOI of 2.5 in a total volume of 150 μl without addition of an rAAV sample.

3 days following infection, cells were lysed in the medium by three freeze-thaw cycles (−80° C. and 37° C.) and the Ad5 wt was inactivated at 56° C. for 35 min. 25 μl of the lysate from each well was transferred to a well of a fresh 96-well plate for the second round of infection. As for the first round of infection, in the second round of infection, cells were seeded in 96-well plates with 1.2e5 vc/cm² and the next day, at 80%+/−10% confluency, cells were infected with the 25 μl lysate from the first round of infection and co-infected with Ad5 wt at an MOI of 2.5 to a total volume of 150 μl per well.

3 days following infection, cells were lysed in the medium by three freeze-thaw cycles (−80° C. and 37° C.) and the Ad5 wt was inactivated at 56° C. for 35 min. 10 μl of each lysate was diluted 1:16 with 150 μl PBS. 5 μl of this dilution was analyzed by qPCR targeting a specific sequence in the cap gene in order to confirm presence of particles containing cap sequence in the rAAV/Ad5 wt infected wells. The qPCR was performed in the same way as the qPCR in Example 2 under the section “Quantification of rAAV vector genomes”, except that the primers used were for the cap sequences. The following primers were used:

Primer cap forward: TACTGAGGGACCATGAAGAC (SEQ ID NO: 9)
Primer cap reverse: GTTTACGGACTCGGAGTATC (SEQ ID NO: 10)

Infections with Ad5 wt only (negative controls) showed no specific product in the qPCR for the cap sequences, while rAAV/Ad5 wt infected wells showed a specific product with Cq (quantification cycle) values <25.

Lysates from the cap positive wells following the second round of infection were harvested, pooled and the cellular debris were removed by centrifugation at 3500×g for 5 min. In order to isolate DNA from packaged AAV particles from the lysates, free cellular DNA was removed by Denarase (Sartorius Stedim Biotech) digest using 75 U/ml for 1.5 h at 37° C. Denarase enzyme was inactivated by subsequent incubation of the lysates at 65° C. for 30 min. The DNA from packaged AAV particles was then isolated using the QIAamp MinElute Virus Spin Kit (Qiagen) with upscaling of the buffer AL volume and ethanol according to the lysate volume before the column was loaded. No carrier RNA was used. In order to enable faster loading of larger volumes to the column a sucking pump was applied. Proteinase K levels and all subsequent steps after column loading were performed according to the manufacturer's instructions without adaption to the lysate volume. DNA was eluted in 30-50 μl RNase-free water and used in Southern blotting, qPCR and PCR analysis as set out below (see Examples 9 and 10).

To serve as a positive control, a few wells were infected with a replication competent AAV containing a 4.7 kb vg which contains functional rep and cap genes flanked by ITRs, rather than being infected with FIX-containing rAAV and co-infected with Ad5 wt. The infection, harvest and analysis of the positive controls was performed in parallel with the rAAV/Ad5 wt infections. This sample is named “rcAAV post-infections” and was used as positive control in the following blotting and PCR based analysis. The sample was included to demonstrate that rcAAV could be generated in the assay system when functional rep and cap genes are present.

### Example 9—Using Southern Blotting to Investigate Replication Competent AAV (rcAAV, which Includes Rep-Deficient rcAAV, Cap-Deficient rcAAV and Pseudo-Wild Type rcAAV) Produced by the Trans-Split Two-Plasmid System

Methods

DNA isolated after two rounds of infection (for enrichment of rcAAV) in Example 8 was analysed by Southern blot with probes targeting rep or cap sequences. The isolated DNA sample is also referred to as the “enriched non-split DNA sample” where the rAAV used for the infection was generated using the non-split system. The isolated DNA sample is also referred to as the “enriched split DNA sample” where the rAAV used for the infection was generated using the trans-split two-plasmid system.

The isolated DNA samples were loaded with 6× loading buffer (18% Ficoll, 300 mM NaOH, 6 mM EDTA, 2.4% SDS, 0.15% Xylene Cyanol) on an 1% alkaline agarose (GeneOn) gel (50 mM NaOH, 1 mM EDTA) and run at 24 V for 17 h at 4° C. Neutralization was performed by washing in 1×TAE buffer (40 mM Tris-Acetate, 1 mM EDTA pH 8.3) for 3×15 min before the gel was stained with 10× GelRed™ (Bovendis) in 1×TAE for 1 h to visualize the DNA marker bands. A picture of the gel was taken on a Fusion Detection System (Vilber) in the UV light mode.

In order to prepare the DNA for transfer, the gel was incubated for 30 min in depurination buffer (0.29 M HCl). After subsequent shaking in denaturation buffer (0.5 M NaOH, 1.5 M NaCl) for another 30 min, the gel was neutralized for 30 min in neutralization buffer (1 M Trizma base, 2 M NaCl). The DNA transfer to an Amersham Hybond N+ blotting membrane (GE Healthcare) was carried out with a Biometra Vacu-Blot Device (Analytik Jena) using 20×SSC buffer (3 M NaCl, 0.3 M Trisodium citrate dihydrate) for 5 h at 100 mbar.

The 604 bp large rep specific probe was produced by PCR amplification of the helper plasmid (P-150). The 2200 bp large cap specific probe was produced by PCR amplification of the P-143 plasmid. The 2200 bp large cap specific probe spans almost all the cap gene sequences. Both PCRs were performed in a conventional PCR cycler system with 40 cycles. The probes were purified using the QIAquick PCR Purification Kit (Qiagen).

Probe labelling and detection was performed by AlkPhos Direct™ labelling and detection system with CDP-Star (GE Healthcare) according to the manufacturer's instructions. Pictures were taken with the Fusion Detection System in chemiluminescence mode for 6 h.

As well as the enriched split DNA samples and enriched non-split DNA samples, several control samples were loaded additionally. Replication competent AAV containing functional rep and cap genes was loaded (named “rcAAV”). Two lanes of “rcAAV” were loaded on either side of the gel. The copy numbers of the two “rcAAV” lanes were 1e7 and 1e6 vg/lane. Different copy numbers were included as a sensitivity control. The “rcAAV” control was also included to indicate the 4.7 kb genome size in wildtype arrangement. Both plasmids used for split sample generation (P-150 helper plasmid; and P-160 vector plasmid) were also digested and a fragment encoding the rep or cap genes was loaded to check for the specificity of the probes. Finally, the sample named “rcAAV post-infections” was prepared and loaded. “rcAAV post-infections” contains the purified DNA from the positive control discussed in Example 8.

Results

Southern blot analysis was performed in order to visualize and compare rcAAV species generated which carry rep and/or cap sequences in the enriched non-split DNA sample and enriched split DNA sample.

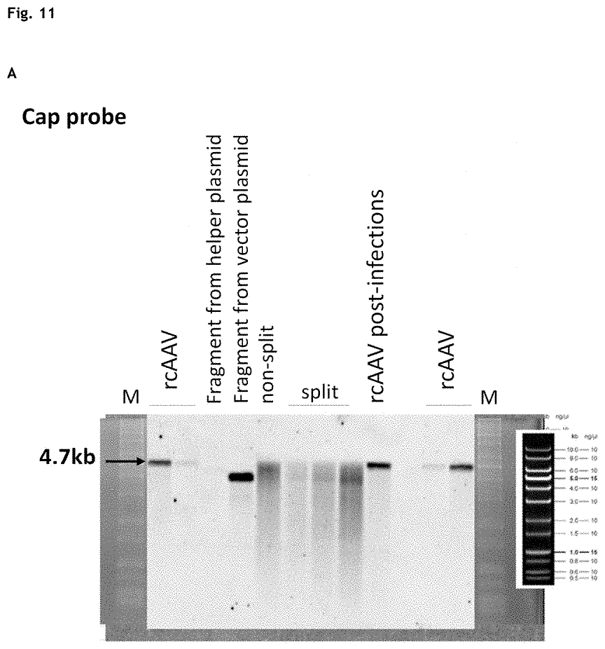

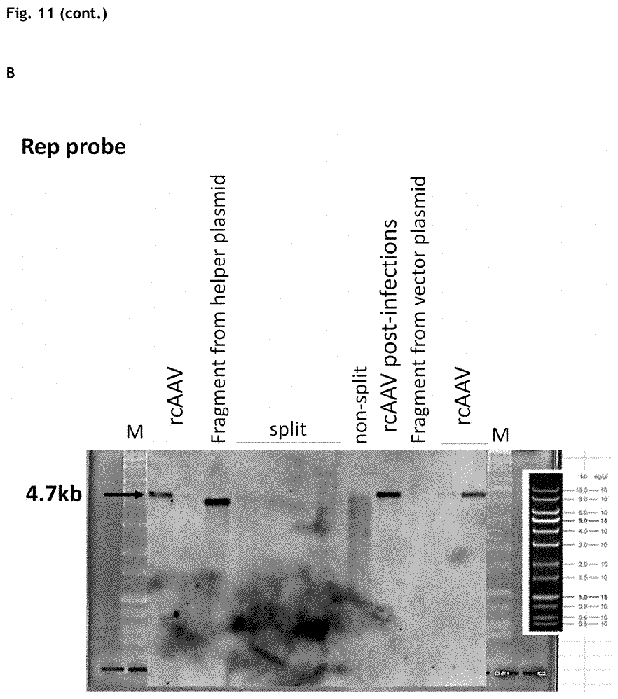

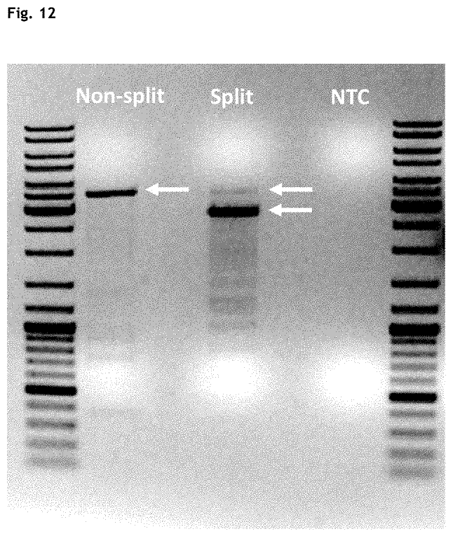

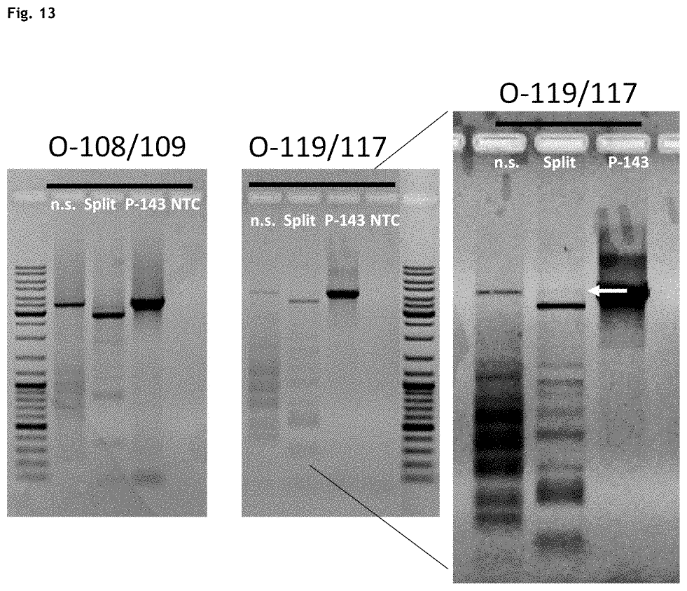

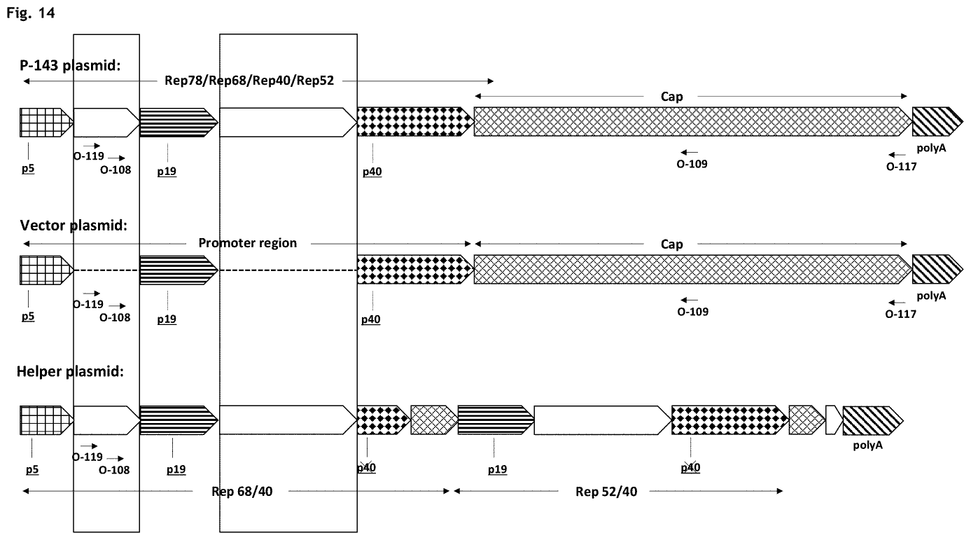

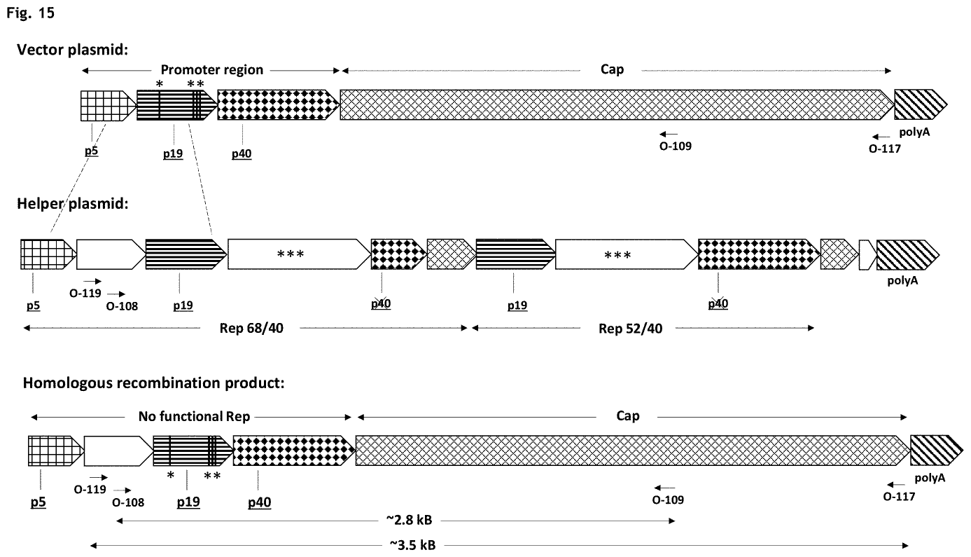

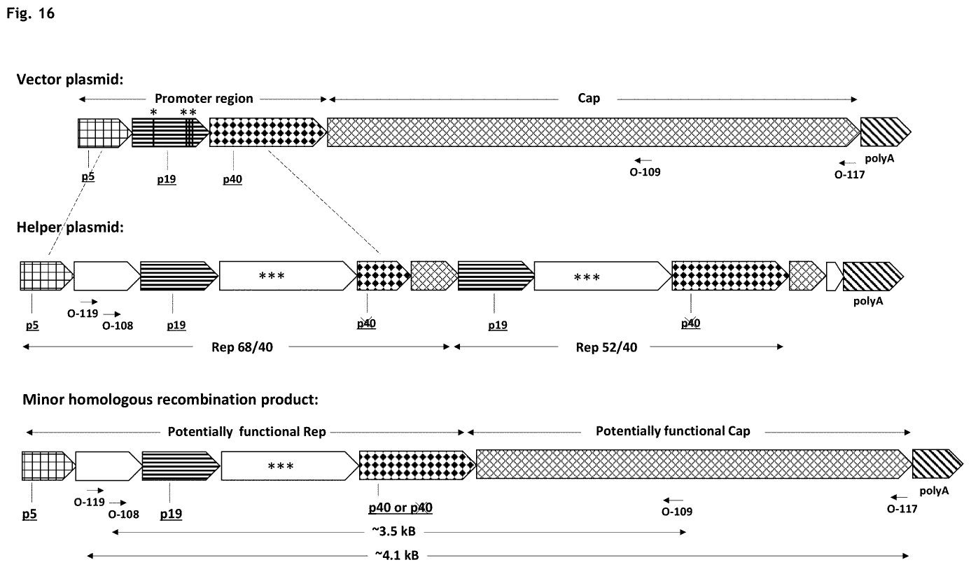

The cap Southern blot (FIG. 11A) showed no distinct bands for both enriched non-split DNA and enriched split DNA samples whereas distinct bands could be seen for the “rcAAV” and “rcAAV post-infections” control samples. Although the non-split and split samples showed mainly a smear over the lane, a diffuse band could be detected for the non-split sample with the strongest signal around 4.7 kb, which would correspond to the wildtype length carrying functional rep and cap genes. In contrast, the split sample (which was loaded in different amounts on the cap blot), showed various species with the strongest signals significantly below the 4.7 kb wildtype length. This suggests that the most prominent species for the split sample (which is being detected after enrichment) contains deletions in the cap genes and/or the rep genes.

On the rep Southern blot (FIG. 11B) all controls showed the expected banding pattern whereas a smear could be detected for the non-split sample and almost no signal could be detected for the split sample. Neither distinct bands nor a strong smear could be detected for the split sample.

Due to the sensitivity limitations of the Southern blot (which were already optimised significantly during the development), PCR based analysis was used to characterize in more detail the species present following enrichment for rcAAV.

### Example 10—Using qPCR to Investigate Replication Competent AAV (rcAAV, which Includes Rep-Deficient rcAAV, Cap-Deficient rcAAV and Pseudo-Wild Type rcAAV) Produced by the Trans-Split Two-Plasmid System

Methods

DNA isolated in Example 8 from equal volumes of lysate following the two rounds of infection (for enrichment of rcAAV) using rAAV generated using the non-split system (also referred to as “enriched non-split DNA sample”) and split system (also referred to as “enriched split DNA sample”) were tested using qPCR to quantify the amount of rep and cap sequences.

The detected copy number of rep and cap in the samples is calculated relative to the plasmid standard range (from 100 to 1×10⁸ copies per qPCR reaction). In short, known quantities of linearised plasmid containing rep (P-150) or containing cap (P-160) were serially diluted to create a standard curve and the copy number of rep and cap in a sample was interpolated from the standard curve.

The qPCR was performed in the same way as the qPCR in Example 2 under the section “Quantification of rAAV vector genomes”, except that the primers used were for rep and cap sequences. The following primers were used:

Primers binding to rep (binding in rep68 exon1, they do not bind in P-160):

Primer rep-fw:
(SEQ ID NO: 7)
5′- CACGTGCATGTGGAAGTAG-3′
Primer rep-rv:
(SEQ ID NO: 8)
5′- CGACTTTCTGACGGAATGG-3′

Primers binding in the cap sequence:

Primer cap-fw:
(SEQ ID NO: 9)
5′-TACTGAGGGACCATGAAGAC-3′
Primer cap-rv:
(SEQ ID NO: 10)
5′-GTTTACGGACTCGGAGTATC-3′

Results

The amount of rep and cap sequences in the enriched non-split DNA sample and the enriched split DNA sample were compared in order to evaluate the molecular arrangement of the enriched rcAAV particles and compare the two plasmid systems.

In the non-split system, both cap and rep sequences originate from the same plasmid (P-143) used for rAAV production and therefore no recombination event is needed to bring rep and cap genes together on one DNA molecule. Therefore, most of the rcAAV generated following the two rounds of infection (for enrichment of rcAAV) are likely to carry cap and rep sequences on the same DNA molecule. This is supported by the results comparing the quantity of rep and cap sequences detected. Almost equal amounts of rep and cap sequences were detected from the enriched non-split DNA sample. The proportion of cap to rep sequences (i.e. the amount of cap divided by rep) was 0.66.

In contrast, the enriched split DNA sample had approximately 40 times more cap sequences than rep (the amount of cap divided by rep was 39.74). Since there is significantly more cap than rep present, it is likely that the cap and rep sequences are not present on the same DNA molecules in the AAV generated. This is consistent with the rep and cap sequences being present on different plasmids in the split plasmid system and, therefore, the requirement that recombination takes place for both rep and cap sequences to be present on the same DNA molecule. Thus, the trans-split two-plasmid system appears to generate fewer AAV species comprising both rep and cap sequences.

Comparing the amount of detected rep copies from both samples, the enriched split DNA sample contained significantly fewer rep species than the enriched non-split DNA sample, with the enriched non-split DNA sample showing a 1.7 fold higher quantity of rep than the enriched split DNA sample. It is important to note that the initial titre of rAAV generated from the split plasmid system and used in the two rounds of infection (for enrichment of rcAAV) was 6.3 times higher than the equivalent initial titre of rAAV generated from the non-split plasmid system. When taking the difference in initial titres into account, it appears that the enriched non-split DNA sample contains 10.7 times more rep sequences than the enriched split DNA sample.

In order to compare the maximum possible amount of rcAAV comprising both rep and cap sequences which may be generated by the split and non-split plasmid systems, the quantities of rep and cap sequences were compared between the enriched split DNA sample and the enriched non-split DNA sample. Where the quantities of rep and cap are not equal, the lower amount between rep and cap indicates the maximum possible number of AAV species which contain rep and cap genes on the same DNA molecule. In the enriched non-split DNA sample, the quantity of cap was lower than rep, and in the enriched split DNA sample, the quantity of rep was lower than cap. The quantity of cap from the enriched non-split DNA samples was 11% higher than the quantity of rep from the enriched split DNA sample. Again, when taking into account that the initial titre of rAAV generated from the split plasmid system and used in the two rounds of infection (for enrichment of rcAAV) was 6.3 times higher than the equivalent initial titre of rAAV generated from the non-split plasmid system, it appears that the enriched non-split DNA sample contains 7 times more AAV species which potentially contain both rep and cap sequences than the enriched split DNA sample.

Similar results to those set out above were also obtained when the quantities of rep and cap were measured by qPCR in the rAAV population generated by the split and non-split systems prior to the two rounds of infection (for enrichment).

### Example 11—Using PCR to Investigate Replication Competent AAV (rcAAV, which Includes Rep-Deficient rcAAV, Cap-Deficient rcAAV and Pseudo-Wild Type rcAAV) Produced by the Trans-Split Two-Plasmid System

Methods

PCR analysis on DNA isolated in Example 8 from equal volumes of lysate following the two rounds of infection (for enrichment of rcAAV) was conducted in order to identify AAV species containing sequences for functional Rep and Cap on the same molecule. Two primer sets were designed.

Primer set O-108/109: Primer O-108 binds in the rep68 gene sequence. This sequence is present in the helper plasmid (P-150) of the trans-split two-plasmid system. Primer O-109 binds in the cap sequence which is present in the vector plasmid (P-160) of the trans-split two-plasmid system (FIG. 15). Generation of an amplification product from the enriched split DNA sample using the O-108/109 primer pair would indicate a recombination event had occurred between the two plasmids (P-150 and P-160) used for rAAV production.

Primer set O-119/117: Primer O-119 binds in the rep68 gene sequence 142 bp 5′ of the above primer O-108. This sequence is present in the helper plasmid (P-150) of the trans-split two-plasmid system. Primer O-117 starts at the stop codon of the cap sequence which is present in the vector plasmid (P-160) of the trans-split two-plasmid system (FIG. 15). Generation of an amplification product from the enriched split DNA sample using the O-119/117 primer pair would also indicate a recombination event had occurred between the two plasmids (P-150 and P-160) used for rAAV production.

The primer set O-119/117 should detect the same recombination events as the primer set O-108/109, but amplifies a larger portion of the rep and cap sequences (including all of the cap gene sequences).

PCR protocol: In a total volume of 25 μl, 12.5 μl of 2× HotStarTaq Plus Master Mix (Qiagen) was mixed with the corresponding primer pair (final concentration 200 nM per primer) and 10 μl of a 1:100 dilution of the sample DNA. P-143 plasmid was used as a positive control with 10⁶ copies per reaction. The PCR was carried out on a Thermal Cycler (BioRad) using the corresponding programs mentioned below. As an additional control, a non-template control (NTC) was run in parallel in which 10 μl nuclease-free water was added to the PCR mixture instead of sample DNA.

For gel analysis, 10 μl of each of the PCR products was mixed with 2 μl 6×DNA loading buffer and loaded on a 1% agarose gel containing ix TAE (40 mM Tris-Acetate, 1 mM EDTA pH 8.3) and 0.005% ROTIGel Stain (Carl Roth). The gel run was performed until optimal marker (PeqLab) separation could be detected. Pictures were taken with a Fusion Detection System (Vilber) in UV mode.

For sequencing, the detected DNA bands were cut out from the agarose gel and purified using the QIAquick gel extraction kit (Qiagen) and sent to GATC (Konstanz).

PCR program for O-108/109 and O-119/117: 95° C. 5 min; 39 cycles (94° C. 30 s, 60° C. 30 s, 72° C. 4 min); 72° C. 10 min; hold

Results

PCR analysis was performed in order to try to detect species which carry rep and cap genes on one molecule from the enriched DNA samples. Therefore, the forward primers (O-108 and O-119) were placed in the rep68 gene and the reverse primers (O-109 and O-117) were placed in the cap gene sequence. Since the forward primers O-108 and O-119 bind in the rep sequence which is originally present in the helper plasmid whereas the reverse primers O-109 and O-117 bind in the cap sequence which is originally present in the vector plasmid, the obtained PCR products for the enriched split DNA sample represent AAV species which arise from a homologous recombination event between the two plasmid sequences of the trans-split system during vector packaging. As the rep and cap sequences are present on same plasmid in the non-split system, homologous recombination is not required in order to obtain PCR products from the enriched non-split DNA sample using these primer pairs.

As shown in FIG. 12, for the enriched non-split DNA sample, a strong PCR product for primer pair O-108/109 was detected with a length of about 3.5 kb. This corresponds to the original (wildtype) arrangement of rep and cap sequences in the P-143 plasmid and was also confirmed by sequencing of the purified PCR product (data not shown). This product is considered to be potentially functional as it contains no deletions in the amplified rep-cap sequences.

For the enriched split DNA sample, the strongest PCR product was detected at a length of about 2.8 kb. Sequencing of this −2.8 kb band confirmed the presence of a recombination product of the vector plasmid (P-160) and helper plasmid (P-150) of the trans-split two plasmid system resulting in a rep-cap molecule with a non-functional rep locus as depicted in FIG. 15. As can be seen in FIG. 15, the recombination product does not have a portion of the rep locus (the box labelled ***) which is present in the original helper plasmid. Thus there are no functional rep genes present (since each rep gene (rep68, rep78, rep40 and rep52) contains the portion of the rep locus which is absent). The detected band at 3.5 kb was very weak and could not be sequenced due to the low abundance but may represent the rep-cap wildtype-like arrangement comparable to the detected species in the enriched non-split DNA sample. FIG. 16 depicts a possible homologous recombination event between the vector plasmid (P-160) and helper plasmid (P-150) of the trans-split two plasmid system leading to this wild type-like arrangement. When the PCR amplification using primer pair O-108/109 was repeated (shown in FIG. 13), the 3.5 kb band was not detected, confirming its low abundance.

Comparing the potentially functional rearranged rep-cap 3.5 kb PCR product of the enriched DNA split sample with the potentially functional rep-cap 3.5 kb PCR product of the enriched non-split DNA sample shows a much stronger product for the non-split system compared to the split system. Thus the potentially wildtype configuration of rep and cap on one DNA molecule in the enriched split DNA sample is low compared to the enriched non-split DNA sample. The homologous recombination required in the split system to arrive at the rearranged potentially wildtype configuration (shown in FIG. 16) appears to be underrepresented in comparison to the homologous recombination required to arrive at the product without functional rep (shown in FIG. 15). When taking into account that the initial titre of rAAV generated from the split plasmid system and used in the two rounds of infection (for enrichment of rcAAV) was 6.3 times higher than the equivalent initial titre of rAAV generated from the non-split plasmid system, it appears that the amount of detected potentially functional rep-cap AAV species in the enriched split DNA sample is significantly lower than the enriched non-split DNA sample. Quantitative signal integration was performed on the 3.5 kb bands shown in FIG. 12 using BiolD software (Vilber). The band intensity for the enriched non-split DNA sample was 7.2 times higher than the band intensity for the enriched split DNA sample. Considering that the non-split 3.5 kb band was already in saturation (and is therefore underestimated) and that the initial titre of rAAV generated from the split plasmid system was 6.3 times higher than the equivalent initial titre of rAAV generated from the non-split plasmid system, it appears that there is at least a 45 times difference in detected potentially functional rep-cap AAV species between the split and non-split samples.

The PCR performed with the 2^{nd} primer pair O-119/117 led to similar results as the PCR performed with the primers O-108/109 (FIG. 13). The PCR amplification using primer pair O-108/109 was repeated and is shown in FIG. 13. The 2^{nd} primer pair O-119/117 amplifies a larger section of the rep-cap sequences including almost the whole rep locus and including all the cap gene sequences. The enriched non-split DNA sample had a strong PCR product with a length of about 4.1 kb for the primer pair O-119/117, which corresponds to the original (wildtype) arrangement of rep and cap sequences in the P-143 plasmid and was also confirmed by sequencing of the purified PCR product (data not shown). For the enriched split DNA sample the strongest PCR product was detected at a length of about 3.5 kb. As for the −2.8 kb product amplified by the O-108/109 primer pair, sequencing of this −3.5 kb band confirmed the presence of a recombination product of the vector plasmid (P-160) and helper plasmid (P-150) of the trans-split two plasmid system resulting in a rep-cap molecule with a non-functional rep locus as depicted in FIG. 15. As for the minor ˜3.5 kb product amplified by the O-108/109 primer pair, the detected band using the O-119/117 primer pair at −4.1 kb was very weak and could not be sequenced due to the low abundance but may represent the rep-cap potentially wildtype-like arrangement comparable to the detected species in the enriched non-split DNA sample. FIG. 16 depicts a possible homologous recombination event between the vector plasmid (P-160) and helper plasmid (P-150) of the trans-split two plasmid system leading to such a wildtype-like arrangement.

1. A two-plasmid system comprising a helper plasmid and a vector plasmid, wherein the helper plasmid comprises at least one rep gene encoding at least one functional Rep protein and does not comprise a cap gene encoding a functional set of Cap proteins.

2. The two-plasmid system of claim 1, wherein the two-plasmid system comprises a molar excess of vector plasmid compared to helper plasmid.

3. The two-plasmid system of claim 1 or 2, wherein the ratio of helper plasmid to vector plasmid is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; or between 1:1.5 and 1:3.

4. The two-plasmid system of any one of claims 1-3, wherein the ratio of helper plasmid to vector plasmid is between 1:2 and 1:4; or around 1:3.

5. The two-plasmid system of any one of claims 1-4, wherein the vector plasmid comprises:
(a) a cap gene encoding at least one functional Cap protein; or
(b) at least one cap gene promoter, a cloning site operably linked to the cap gene promoter, and an expression cassette flanked on at least one side by an ITR;
wherein the vector plasmid does not comprise a rep gene encoding a functional Rep protein and the expression cassette comprises a transgene operably linked to at least one regulatory control element.

6. The two-plasmid system of any one of claims 1-5, wherein the at least one rep gene comprises a gene encoding a functional Rep 52 protein, at least one gene encoding a functional Rep 40 protein, and a gene encoding a functional Rep 68 protein.

7. The two-plasmid system of any one of claims 1-6, wherein the at least one rep gene does not comprise a functional internal p40 promoter.

8. The two-plasmid system of any one of claims 1-7, wherein:
(i) the at least one rep gene comprises a C nucleotide at a position corresponding to position 1823 of SEQ ID NO: 1; and/or
(ii) the helper plasmid does not comprise a contiguous stretch of exclusively cap gene sequence of more than 250 nucleotides, more than 100 nucleotides, or more than 60 nucleotides.

9. The two-plasmid system of any one of claims 1-8, wherein the helper plasmid comprises a portion of cap gene sequence, and the portion of cap gene sequence does not encode a functional set of Cap proteins.

10. The two-plasmid system of any one of claims 1-9, wherein the helper plasmid comprises at least one helper virus gene, optionally wherein:
(i) the at least one helper virus gene is an adenovirus gene, optionally an Adenovirus 5 or Adenovirus 2 gene; and/or
(ii) the at least one helper virus gene comprises a VA nucleic acid encoding functional VA RNA I and II, an E2A gene encoding a functional E2A protein and an E4 gene encoding a functional E4 protein.

11. The two-plasmid system of claim 10, wherein:
(i) the at least one helper virus gene comprises a VA nucleic acid encoding functional VA RNA I and II, an E2A gene encoding a functional E2A protein and an E4 gene encoding a functional E4 protein, and wherein the E4 gene is not located between the VA nucleic acid and the E2A gene; and/or
(ii) the helper plasmid is less than 25000 bp, less than 20000 bp, less than 15000 bp, less than 14500 bp, between 10000 bp and 25000 bp, between 10000 bp and 20000 bp, between 12000 bp and 15000 bp, or around 14021 bp in length.

12. The two-plasmid system of any one of claims 1-11, wherein:
(i) the helper plasmid and/or the vector plasmid does not comprise an artificial Rep binding site; and/or
(ii) the helper plasmid and/or the vector plasmid comprises a plasmid backbone, and the plasmid backbone does not comprise an artificial Rep binding site.

13. The two-plasmid system of any one of claims 1-12, wherein the vector plasmid comprises a cap gene and further comprises an expression cassette flanked on at least one side by an ITR.

14. The two-plasmid system of any one of claims 1-13, wherein the vector plasmid comprises a cap gene and the cap gene encodes a Cap protein selected from the group of AAV serotypes consisting of serotypes 2, 5, 8, 9, and Mut C (SEQ ID NO: 3 from WO 2016/181123).

15. The two-plasmid system of any one of claims 1-14, wherein:
(i) the vector plasmid does not comprise any dispensable translation initiation codons; and/or
(ii) the vector plasmid does not comprise any dispensable translation initiation codons, wherein the vector plasmid comprises a promoter region comprising one or more promoters, and the promoter region does not comprise ATG or GTG codons, optionally wherein the promoter region comprises p5, p19 and p40 promoters, and wherein ATG or GTG codons at one or more positions corresponding to positions (a) 321-323, (b) 766-768, (c) 955-957, (d) 993-995 and (e) 1014-1016 of SEQ ID NO: 1 are absent or mutated.

16. The two-plasmid system of any one of claims 1-15, wherein the vector plasmid comprises a backbone less than 4000 nucleotides, less than 3500 nucleotides, less than 3000 nucleotides, or less than 2500 nucleotides in length.

17. Use of the two-plasmid system, the helper plasmid or the vector plasmid as defined in any one of claims 1-16 for producing a recombinant AAV preparation:
(a) having a desired ratio of full to total particles; and/or
(b) at a high or desired yield.

18. Use of the two-plasmid system, the helper plasmid or the vector plasmid as defined in any one of claims 1-16 for:
(e) controlling or maximising the ratio of full to total particles produced during recombinant AAV production; and/or
(f) increasing, optimising or maximising the yield of recombinant AAV produced during recombinant AAV production.

19. The use of claim 17 or 18, wherein the use comprises transfecting a host cell with the two-plasmid system, the helper plasmid or the vector plasmid of any one of claims 1-16 and culturing the host cell under conditions suitable for recombinant AAV production.

20. A method for controlling or maximising the ratio of full to total particles produced during recombinant AAV production comprising:
(d) obtaining the two-plasmid system, the helper plasmid or the vector plasmid as defined in any one of claims 1-16;
(e) transfecting a host cell with the two-plasmid system, the helper plasmid or the vector plasmid as defined in any one of claims 1-16; and
(f) culturing the host cell under conditions suitable for recombinant AAV production.

21. The method of claim 20, further comprising a step of harvesting the recombinant AAV to provide a recombinant AAV preparation comprising a desired ratio of full to total particles.

22. The method of claim 20 or 21, wherein the method is a method for producing a recombinant AAV preparation at a high or desired yield.

23. A method for increasing, optimising or maximising the yield of recombinant AAV produced during recombinant AAV production comprising:
(d) obtaining the two-plasmid system, the helper plasmid or the vector plasmid as defined in any one of claims 1-16;
(e) transfecting a host cell with the two-plasmid system, the helper plasmid or the vector plasmid as defined in any one of claims 1-16; and
(f) culturing the host cell under conditions suitable for recombinant AAV production.

24. The use or method of any one of claims 17, 19 or 22, wherein the high or desired yield is a yield that is at least 2-fold, at least 4-fold, at least 5-fold, or at least 6-fold higher than the yield achieved using an equivalent method with a ratio of helper plasmid to vector plasmid of 1.8:1.

25. The use or method of any one of claims 17, 19, 21, 22 or 24, wherein the desired ratio of full to total particles is a ratio of full to total particles that is at least 20% or at least 30% of the ratio of full to total particles achieved using an equivalent method with a ratio of helper plasmid to vector plasmid of 1.8:1.

26. The use or method of any one of claims 17-19, 20, 21, 22, 24 or 25, wherein the ratio of helper plasmid to vector plasmid is between 3:1 to 1:10, between 1.5:1 and 1:9, between 1.4:1 and 1:8, between 1.3:1 and 1:7; between 1.2:1 and 1:6; between 1.1:1 and 1:5; between 1:1 and 1:4; or between 1:1.5 and 1:3.

27. The use or method of any one of claims 17-19, 20, 21, 22, 24, 25 or 26, wherein the ratio of helper plasmid to vector plasmid is between 1:2 and 1:4; or around 1:3.


SEQUENCE LISTING (10 sequences)

SEQ ID NO: 1 - DNA, 4679 bp, Adeno associated virus type 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 2 - DNA, 35938 bp, adenovirus type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaaacgaatagatgccatgaaccacccaactttccccgcgcccgctatgcttccactgcaacaagttgttgccggcggctttgtcccagccaatcagcctcgccccacttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 3 - DNA, 35937 bp, adenovirus type 2
catcatcataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgccggatgtggtaaaagtgacgtttttggtgtgcgccggtgtatacgggaagtgacaattttcgcgcggttttaggcggatgttgtagtaaatttgggcgtaaccaagtaatgtttggccattttcgcgggaaaactgaataagaggaagtgaaatctgaataattctgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgcgcagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgagtctgtaatgttggcggtgcaggaagggattgacttattcacttttccgccggcgcccggttctccggagccgcctcacctttcccggcagcccgagcagccggagcagagagccttgggtccggtttctatgccaaaccttgtgccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtcggtgatagagtggtgggtttggtgtggtaatttttttttaatttttacagttttgtggtttaaagaattttgtattgtgattttttaaaaggtcctgtgtctgaacctgagcctgagcccgagccagaaccggagcctgcaagacctacccggcgtcctaaattggtgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagtctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcctcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggtggtgagacacaagaatcgcctgctactgttgtcttccgtccgcccggcaataataccgacggaggagcaacagcaggaggaagccaggcggcggcggcggcaggagcagagcccatggaacccgagagccggcctggaccctcgggaatgaatgttgtacaggtggctgaactgtttccagaactgagacgcattttaaccattaacgaggatgggcaggggctaaagggggtaaagagggagcggggggcttctgaggctacagaggaggctaggaatctaacttttagcttaatgaccagacaccgtcctgagtgtgttacttttcagcagattaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagattagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtgaggtttactggtcccaattttagcggtacggttttcctggccaataccaatcttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctgtttgaaaggtgtaccttgggtatcctgtctgagggtaactccagggtgcgccacaatgtggcctccgactgtggttgcttcatgctagtgaaaagcgtggctgtgattaagcataacatggtgtgtggcaactgcgaggacagggcctctcagatgctgacctgctcggacggcaactgtcacttgctgaagaccattcacgtagccagccactctcgcaaggcctggccagtgtttgagcacaacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtctcatgtagttttgtatctgttttgcagcagccgccgccatgagcgccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaccagactctgtttggattttgatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccgagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaaccgtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgacttgcatgttttccctgaccaaatgcgccagaaggcgctcgccgcccagcgatagcagttcttgcaaggaagcaaagtttttcaacggtttgaggccgtccgccgtaggcatgcttttgagcgtttgaccaagcagttccaggcggtcccacagctcggtcacgtgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttaagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacgaaggctcgcgtccaggccagcacgaaggaggctaagtgggaggggtagcggtcgttgtccactagggggtccactcgctccagggtgtgaagacacatgtcgccctcttcggcatcaaggaaggtgattggtttataggtgtaggccacgtgaccgggtgttcctgaaggggggctataaaagggggtgggggcgcgttcgtcctcactctcttccgcatcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgaacagaatggcggtagtgggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctaaccatgactttgaggtactggtatttgaagtcagtgtcgtcgcatccgccctgctcccagagcaaaaagtccgtgcgctttttggaacgcgggtttggcagggcgaaggtgacatcgttgaaaagtatctttcccgcgcgaggcataaagttgcgtgtgatgcggaagggtcccggcacctcggaacggttgttaattacctgggcggcgagcacgatctcgtcgaagccgttgatgttgtggcccacgatgtaaagttccaagaagcgcggggtgcccttgatggagggcaattttttaagttcctcgtaggtgagctcctcaggggagctgagcccgtgttctgacagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggtccacggctaggtctcgcgcggcggtcaccagaggctcatctccgccgaacttcataaccagcatgaagggcacgagctgcttcccaaaggcccccatccaagtataggtctctacatcgtaggtgacaaagagacgctcggtgcgaggatgcgagccgatcgggaagaactggatctcccgccaccagttggaggagtggctgttgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttatggtggatcggaccaccacgccgcgcgagcccaaagtccagatgtccgcgcgcggcggtcggagcttgatgacaacatcgcgcagatgggagctgtccatggtctggagctcccgcggcgacaggtcaggcgggagctcctgcaggtttacctcgcatagccgggtcagggcgcgggctaggtccaggtgatacctgatttccaggggctggttggtggcggcgtcgatgacttgcaagaggccgcatccccgcggcgcgactacggtaccgcgcggcgggcggtgggccgcgggggtgtccttggatgatgcatctaaaagcggtgacgcgggcgggcccccggaggtagggggggctcgggacccgccgggagagggggcaggggcacgtcggcgccgcgcgcgggcaggagctggtgctgcgcgcggaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatttcggccatgaactgctcgatctcttcctcctggagatctccgcgtccggctcgctccacggtggcggcgaggtcgttggagatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcaatctcctcttccataagggcctccccttcttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccgtgcggcagggatacggcgctaacgatgcatctcaacaattgttgtgtaggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctacggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactgatatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccgggcccggcactacctggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccacgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagctttaacaaccacgtgcgcacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacattctgcagagcatagtggtgcaggagcgcagcttgagcctggctgacaaggtggccgccattaactattccatgctcagtctgggcaagttttacgcccgcaagatataccataccccttacgttcccatagacaaggaggtaaagatcgaggggttctacatgcgcatggcgttgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgtaaccctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgatgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtccgggccagactattttttccagaccagtagacaaggcctgcagaccgtaaacctgagccaggctttcaagaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctgaactacctgctgaccaaccggcggcagaagatcccctcgttgcacagtttaaacagcgaggaggagcgcatcttgcgctatgtgcagcagagcgtgagccttaacctgatgcgcgacggggtaacgcccagcgtggcgctggacatgaccgcgcgcaacatggaaccgggcatgtatgcctcaaaccggccgtttatcaatcgcctaatggactacttgcatcgcgcggccgccgtgaaccccgagtatttcaccaatgccatcttgaacccgcactggctaccgccccctggtttctacaccgggggatttgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgcgagtagcccatttccaagcttgatagggtcttttaccagcactcgcaccacccgcccgcgcctgctgggcgaggaggagtacctaaacaactcgctgctgcagccgcagcgcgaaaagaacctgcctccggcatttcccaacaacgggatagagagcctagtggacaagatgagtagatggaagacgtatgcgcaggagcacagggatgtgcccggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaaaaaagcatgatgcaaaataaaaaactcaccaaggccatggcaccgagcgttggttttcttgtattccccttagtatgcagcgcgcggcgatgtatgaggaaggtcctcctccctcctacgagagcgtggtgagcgcggcgccagtggcggcggcgctgggttcccccttcgatgctcccctggacccgccgtttgtgcctccgcggtacctgcggcctaccggggggagaaacagcatccgttactctgagttggcacccctattcgacaccacccgtgtgtaccttgtggacaacaagtcaacggatgtggcatccctgaactaccagaacgaccacagcaactttctaaccacggtcattcaaaacaatgactacagcccgggggaggcaagcacacagaccatcaatcttgacgaccgttcgcactggggcggcgacctgaaaaccatcctgcataccaacatgccaaatgtgaacgagttcatgtttaccaataagtttaaggcgcgggtgatggtgtcgcgctcgcttactaaggacaaacaggtggagctgaaatatgagtgggtggagttcacgctgcccgagggcaactactccgagaccatgaccatagaccttatgaacaacgcgatcgtggagcactacttgaaagtgggcaggcagaacggggttctggaaagcgacatcggggtaaagtttgacacccgcaacttcagactggggtttgacccagtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgacctggagggtggtaacattcccgcactgttggatgtggacgcctaccaggcaagcttaaaagatgacaccgaacagggcggggatggcgcaggcggcggcaacaacagtggcagcggcgcggaagagaactccaacgcggcagccgcggcaatgcagccggtggaggacatgaacgatcatgccattcgcggcgacacctttgccacacgggcggaggagaagcgcgctgaggccgaggcagcggcagaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccgggatccgctcatggaccctcctttgcactcctgacgtaacctgcggctcggagcaggtctactggtcgttgccagacatgatgcaagaccccgtgaccttccgctccacgagccagatcagcaactttccggtggtgggcgccgagctgttgcccgtgcactccaagagcttctacaacgaccaggccgtctactcccagctcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaaacatgtccatccttatatcgcccagcaataacacaggctggggcctgcgcttcccaagcaagatgtttggcggggcaaagaagcgctccgaccaacacccagtgcgcgtgcgcgggcactaccgcgcgccctggggcgcgcacaaacgcggccgcactgggcgcaccaccgtcgatgacgccattgacgcggtggtggaggaggcgcgcaactacacgcccacgccgccaccagtgtccacagtggacgcggccattcagaccgtggtgcgcggagcccggcgttatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtactgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgatgaacttgacgacgaggtggaactgctgcacgcaaccgcgcccaggcggcgggtacagtggaaaggtcgacgcgtaagacgtgttttgcgacccggcaccaccgtagtttttacgcccggtgagcgctccacccgcacctacaagcgcgtgtatgatgaggtgtacggcgacgaggacctgcttgagcaggccaacgagcgcctcggggagtttgcctacggaaagcggcataaggacatgttggcgttgccgctggacgagggcaacccaacacctagcctaaagcccgtgacactgcagcaggtgctgcccacgcttgcaccgtccgaagaaaagcgcggcctaaagcgcgagtctggtgacttggcacccaccgtgcagctgatggtacccaagcgccagcgactggaagatgtcttggaaaaaatgaccgtggagcctgggctggagcccgaggtccgcgtgcggccaatcaagcaggtggcaccgggactgggcgtgcagaccgtggacgttcagatacccaccaccagtagcactagtattgccactgccacagagggcatggagacacaaacgtccccggttgcctcggcggtggcagatgccgcggtgcaggcggccgctgcggccgcgtccaaaacctctacggaggtgcaaacggacccgtggatgtttcgcgtttcagccccccggcgcccgcgccgttccaggaagtacggcaccgccagcgcactactgcccgaatatgccctacatccttccatcgcgcctacccccggctatcgtggctacacctaccgccccagaagacgagcgactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggagtctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccattaagaactatggcagcaaggcctggaacagcagcacaggccagatgctgagggacaagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcggcccgacagggaagaaactctggtgacgcaaatagatgagcctccctcgtacgaggaggcactaaagcaaggcctgcccaccacccgtcccatcgcgcccatggctaccggagtgctgggccagcacacacctgtaacgctggacctgcctccccccgctgacacccagcagaaacctgtgctgccagggccgtccgccgttgttgtaacccgccctagccgcgcgtccctgcgccgtgccgccagcggtccgcgatcgatgcggcccgtagccagtggcaactggcaaagcacactgaacagcatcgtgggtctgggggtgcaatccctgaagcgccgacgatgcttctaaatagctaacgtgtcgtatgtgtcatgtatgcgtccatgtcgccgccagaggagctgctgagccgccgtgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcacctacgcacgacgtaaccacagaccggtcccagcgtttgacgctgcggttcatccctgtggaccgcgaggataccgcgtactcgtacaaagcgcggttcaccctggctgtgggtgacaaccgtgtgcttgatatggcttccacgtactttgacatccgcggcgtgctggacagggggcctacttttaagccctactccggcactgcctacaacgctctagctcccaagggcgctcctaactcctgtgagtgggaacaaaccgaagatagcggccgggcagttgccgaggatgaagaagaggaagatgaagatgaagaagaggaagaagaagagcaaaacgctcgagatcaggctactaagaaaacacatgtctatgcccaggctcctttgtctggagaaacaattacaaaaagcgggctacaaataggatcagacaatgcagaaacacaagctaaacctgtatacgcagatccttcctatcaaccagaacctcaaattggcgaatctcagtggaacgaagctgatgctaatgcggcaggagggagagtgcttaaaaaaacaactcccatgaaaccatgctatggatcttatgccaggcctacaaatccttttggtggtcaatccgttctggttccggatgaaaaaggggtgcctcttccaaaggttgacttgcaattcttctcaaatactacctctttgaacgaccggcaaggcaatgctactaaaccaaaagtggttttgtacagtgaagatgtaaatatggaaaccccagacacacatctgtcttacaaacctggaaaaggtgatgaaaattctaaagctatgttgggtcaacaatctatgccaaacagacccaattacattgctttcagggacaattttattggcctaatgtattataacagcactggcaacatgggtgttcttgctggtcaggcatcgcagctaaatgccgtggtagatttgcaagacagaaacacagagctgtcctatcaactcttgcttgattccataggtgatagaaccagatatttttctatgtggaatcaggctgtagacagctatgatccagatgttagaatcattgaaaaccatggaactgaggatgaattgccaaattattgttttcctcttgggggtattggggtaactgacacctatcaagctattaaggctaatggcaatggctcaggcgataatggagatactacatggacaaaagatgaaacttttgcaacacgtaatgaaataggagtgggtaacaactttgccatggaaattaacctaaatgccaacctatggagaaatttcctttactccaatattgcgctgtacctgccagacaagctaaaatacaaccccaccaatgtggaaatatctgacaaccccaacacctacgactacatgaacaagcgagtggtggctcccgggcttgtagactgctacattaaccttggggcgcgctggtctctggactacatggacaacgttaatccctttaaccaccaccgcaatgcgggcctccgttatcgctccatgttgttgggaaacggccgctacgtgccctttcacattcaggtgccccaaaagttttttgccattaaaaacctcctcctcctgccaggctcatatacatatgaatggaacttcaggaaggatgttaacatggttctgcagagctctctgggaaacgatcttagagttgacggggctagcattaagtttgacagcatttgtctttacgccaccttcttccccatggcccacaacacggcctccacgctggaagccatgctcagaaatgacaccaacgaccagtcctttaatgactacctttccgccgccaacatgctataccccatacccgccaacgccaccaacgtgcccatctccatcccatcgcgcaactgggcagcatttcgcggttgggccttcacacgcttgaagacaaaggaaaccccttccctgggatcaggctacgacccttactacacctactctggctccataccataccttgacggaaccttctatcttaatcacacctttaagaaggtggccattacctttgactcttctgttagctggccgggcaacgaccgcctgcttactcccaatgagtttgagattaaacgctcagttgacggggagggctacaacgtagctcagtgcaacatgaccaaggactggttcctggtgcagatgttggccaactacaatattggctaccagggcttctacattccagaaagctacaaggaccgcatgtactcgttcttcagaaacttccagcccatgagccggcaagtggttgacgatactaaatacaaggagtatcagcaggttggaattcttcaccagcataacaactcaggattcgtaggctacctcgctcccaccatgcgcgagggacaggcttaccccgccaacgtgccctacccactaataggcaaaaccgcggttgacagtattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgagaccgtgtacctgcgcacgcccttctcggccggcaacgccacaacataaaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcgctcaaaaacatgctacctctttgagccctttggcttttctgaccaacgactcaagcaggtttaccagtttgagtacgagtcactcctgcgccgtagcgccattgcttcttcccccgaccgctgtataacgctggaaaagtccacccaaagcgtgcaggggcccaactcggccgcctgtggactattctgctgcatgtttctccacgcctttgccaactggccccaaactcccatggatcacaaccccaccatgaaccttattaccggggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgaaaatcagagggcgaggtattcagctcaacgacgagtcggtgagctcctctcttggtctccgtccggacgggacatttcagatcggcggcgctggccgctcttcatttacgccccgtcaggcgatcctaactctgcagacctcgtcctcggagccgcgctccggaggcattggaactctacaatttattgaggagttcgtgccttcggtttacttcaaccccttttctggacctcccggccactacccggaccagtttattcccaactttgacgcggtgaaagactcggcggacggctacgactgaatgaccagtggagaggcagagcgactgcgcctgacacacctcgaccactgccgccgccacaagtgctttgcccgcggctccggtgagttttgttactttgaattgcccgaagagcatatcgagggcccggcgcacggcgtccggctcaccacccaggtagagcttacacgtagcctgattcgggagtttaccaagcgccccctgctagtggagcgggagcggggtccctgtgttctgaccgtggtttgcaactgtcctaaccctggattacatcaagatctttgttgtcatctctgtgctgagtataataaatacagaaattagaatctactggggctcctgtcgccatcctgtgaacgccaccgtttttacccacccaaagcagaccaaagcaaacctcacctccggtttgcacaagcgggccaataagtaccttacctggtactttaacggctcttcatttgtaatttacaacagtttccagcgagacgaagtaagtttgccacacaaccttctcggcttcaactacaccgtcaagaaaaacaccaccaccaccaccctcctcacctgccgggaacgtacgagtgcgtcaccggttgctgcgcccacacctacagcctgagcgtaaccagacattactcccatttttccaaaacaggaggtgagctcaactcccggaactcaggtcaaaaaagcattttgcggggtgctgggattttttaattaagtatatgagcaattcaagtaactctacaagcttgtctaatttttctggaattggggtcggggttatccttactcttgtaattctgtttattcttatactagcacttctgtgccttagggttgccgcctgctgcacgcacgtttgtacctattgtcagctttttaaacgctgggggcaacatccaagatgaggtacatgattttaggcttgctcgcccttgcggcagtctgcagcgctgccaaaaaggttgagtttaaggaaccagcttgcaatgttacatttaaatcagaagctaatgaatgcactactcttataaaatgcaccacagaacatgaaaagcttattattcgccacaaagacaaaattggcaagtatgctgtatatgctatttggcagccaggtgacactaacgactataatgtcacagtcttccaaggtgaaaatcgtaaaacttttatgtataaatttccattttatgaaatgtgcgatattaccatgtacatgagcaaacagtacaagttgtggcccccacaaaagtgtttagagaacactggcaccttttgttccaccgctctgcttattacagcgcttgctttggtatgtaccttactttatctcaaatacaaaagcagacgcagttttattgatgaaaagaaaatgccttgattttccgcttgcttgtattcccctggacaatttactctatgtgggatatgctccaggcgggcaagattatacccacaaccttcaaatcaaactttcctggacgttagcgcctgatttctgccagcgcctgcactgcaaatttgatcaaacccagcttcagcttgcctgctccagagatgaccggctcaaccatcgcgcccacaacggactatcgcaacaccactgctaccggactaacatctgccctaaatttaccccaagttcatgcctttgtcaatgactgggcgagcttggacatgtggtggttttccatagcgcttatgtttgtttgccttattattatgtggcttatttgttgcctaaagcgcagacgcgccagaccccccatctataggcctatcattgtgctcaacccacacaatgaaaaaattcatagattggacggtctgaaaccatgttctcttcttttacagtatgattaaatgagacatgattcctcgagttcttatattattgacccttgttgcgcttttctgtgcgtgctctacattggccgcggtcgctcacatcgaagtagattgcatcccacctttcacagtttacctgctttacggatttgtcacccttatcctcatctgcagcctcgtcactgtagtcatcgccttcattcagttcattgactgggtttgtgtgcgcattgcgtacctcaggcaccatccgcaatacagagacaggactatagctgatcttctcagaattctttaattatgaaacggagtgtcatttttgttttgctgattttttgcgccctacctgtgctttgctcccaaacctcagcgcctcccaaaagacatatttcctgcagattcactcaaatatggaacattcccagctgctacaacaaacagagcgatttgtcagaagcctggttatacgccatcatctctgtcatggttttttgcagtaccatttttgccctagccatatatccataccttgacattggctggaatgccatagatgccatgaaccaccctactttcccagtgcccgctgtcataccactgcaacaggttattgccccaatcaatcagcctcgccccccttctcccacccccactgagattagctactttaatttgacaggtggagatgactgaatctctagatctagaattggatggaattaacaccgaacagcgcctactagaaaggcgcaaggcggcgtccgagcgagaacgcctaaaacaagaagttgaagacatggttaacctacaccagtgtaaaagaggtatcttttgtgtggtcaagcaggccaaacttacctacgaaaaaaccactaccggcaaccgcctcagctacaagctacccacccagcgccaaaaactggtgcttatggtgggagaaaaacctatcaccgtcacccagcactcggcagaaacagagggctgcctgcacttcccctatcagggtccagaggacctctgcactcttattaaaaccatgtgtggtattagagatcttattccattcaactaacataaacacacaataaattacttacttaaaatcagtcagcaaatctttgtccagcttattcagcatcacctcctttccttcctcccaactctggtatctcagccgccttttagctgcaaactttctccaaagtttaaatgggatgtcaaattcctcatgttcttgtccctccgcacccactatcttcatattgttgcagatgaaacgcgccagaccgtctgaagacaccttcaaccccgtgtatccatatgacacagaaaccgggcctccaactgtgccctttcttacccctccatttgtttcacccaatggtttccaagaaagtccccctggagttctctctctacgcgtctccgaacctttggacacctcccacggcatgcttgcgcttaaaatgggcagcggtcttaccctagacaaggccggaaacctcacctcccaaaatgtaaccactgttactcagccacttaaaaaaacaaagtcaaacataagtttggacacctccgcaccacttacaattacctcaggcgccctaacagtggcaaccaccgctcctctgatagttactagcggcgctcttagcgtacagtcacaagccccactgaccgtgcaagactccaaactaagcattgctactaaagggcccattacagtgtcagatggaaagctagccctgcaaacatcagcccccctctctggcagtgacagcgacacccttactgtaactgcatcacccccgctaactactgccacgggtagcttgggcattaacatggaagatcctatttatgtaaataatggaaaaataggaattaaaataagcggtcctttgcaagtagcacaaaactccgatacactaacagtagttactggaccaggtgtcaccgttgaacaaaactcccttagaaccaaagttgcaggagctattggttatgattcatcaaacaacatggaaattaaaacgggcggtggcatgcgtataaataacaacttgttaattctagatgtggattacccatttgatgctcaaacaaaactacgtcttaaactggggcagggacccctgtatattaatgcatctcataacttggacataaactataacagaggcctatacctttttaatgcatcaaacaatactaaaaaactggaagttagcataaaaaaatccagtggactaaactttgataatactgccatagctataaatgcaggaaagggtctggagtttgatacaaacacatctgagtctccagatatcaacccaataaaaactaaaattggctctggcattgattacaatgaaaacggtgccatgattactaaacttggagcgggtttaagctttgacaactcaggggccattacaataggaaacaaaaatgatgacaaacttaccctgtggacaaccccagacccatctcctaactgcagaattcattcagataatgactgcaaatttactttggttcttacaaaatgtgggagtcaagtactagctactgtagctgctttggctgtatctggagatctttcatccatgacaggcaccgttgcaagtgttagtatattccttagatttgaccaaaacggtgttctaatggagaactcctcacttaaaaaacattactggaactttagaaatgggaactcaactaatgcaaatccatacacaaatgcagttggatttatgcctaaccttctagcctatccaaaaacccaaagtcaaactgctaaaaataacattgtcagtcaagtttacttgcatggtgataaaactaaacctatgatacttaccattacacttaatggcactagtgaatccacagaaactagcgaggtaagcacttactctatgtcttttacatggtcctgggaaagtggaaaatacaccactgaaacttttgctaccaactcttacaccttctcctacattgcccaggaataaagaatcgtgaacctgttgcatgttatgtttcaacgtgtttatttttcaattgcagaaaatttcaagtcatttttcattcagtagtatagccccaccaccacatagcttatattgatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcgcttaagttcatgtcgctgtccagctgctgagccacaggctgctgtccaacttgcggttgctcaacgggcggcgaaggggaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgtcgcttagctcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaactgccctcacgtccaagtggacgtaaaggctaaacccttcagggtgaatctcctctataaacattccagcaccttcaaccatgcccaaataattttcatctcgccaccttatcaatatgtctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagcccctatgtaagcttgttgcatgggcggcgatataaaatgcaaggtgctgctcaaaaaatcaggcaaagcctcgcgcaaaaaagcaagcacatcgtagtcatgctcatgcagataaaggcaggtaagttccggaaccaccacagaaaaagacaccatttttctctcaaacatgtctgcgggttcctgcattaaacacaaaataaaataacaaaaaaaaacatttaaacattagaagcctgtcttacaacaggaaaaacaacccttataagcataagacggactacggccatgccggcgtgaccgtaaaaaaactggtcaccgtgattaaaaagcaccaccgacagttcctcggtcatgtccggagtcataatgtaagactcggtaaacacatcaggttggttaacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaaacctattaaaaaacaccactcgacacggcaccagctcaatcagtcacagtgtaaaaagggccaagtacagagcgagtatatataggactaaaaaatgacgtaacggttaaagtccacaaaaaacacccagaaaaccgcacgcgaacctacgcccagaaacgaaagccaaaaaacccacaacttcctcaaatcttcacttccgttttcccacgatacgtcacttcccattttaaaaaaactacaattcccaatacatgcaagttactccgccctaaaacctacgtcacccgccccgttcccacgccccgcgccacgtcacaaactccaccccctcattatcatattggcttcaatccaaaataaggtatattatgatgatg

SEQ ID NO: 4 - DNA, 8342 bp, Artificial Sequence
  Helper gene region
ccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttaaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatgtcgacaacgttggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaagtcgacagcgaagatcagcttcggcgcacgctggaagacgcggaggctctcttcagtaaatactgcgcgctgactcttaaggactagtttcgcgccctttctcaaatttaagcgcgaaaactacgtcatctccagcgttcgaacttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtggatccgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgctggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggggatcccaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagattaattaaactacaattcccaacacatacaagttactccgccctaaaacctacgtcacccgc

SEQ ID NO: 5 - DNA, 35934 bp, adenovirus type 5
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacgagtgcgtcaccggccgctgcaccacacctaccgcctgaccgtaaaccagactttttccggacagacctcaataactctgtttaccagaacaggaggtgagcttagaaaacccttagggtattaggccaaaggcgcagctactgtggggtttatgaacaattcaagcaactctacgggctattctaattcaggtttctctagaatcggggttggggttattctctgtcttgtgattctctttattcttatactaacgcttctctgcctaaggctcgccgcctgctgtgtgcacatttgcatttattgtcagctttttaaacgctggggtcgccacccaagatgattaggtacataatcctaggtttactcacccttgcgtcagcccacggtaccacccaaaaggtggattttaaggagccagcctgtaatgttacattcgcagctgaagctaatgagtgcaccactcttataaaatgcaccacagaacatgaaaagctgcttattcgccacaaaaacaaaattggcaagtatgctgtttatgctatttggcagccaggtgacactacagagtataatgttacagttttccagggtaaaagtcataaaacttttatgtatacttttccattttatgaaatgtgcgacattaccatgtacatgagcaaacagtataagttgtggcccccacaaaattgtgtggaaaacactggcactttctgctgcactgctatgctaattacagtgctcgctttggtctgtaccctactctatattaaatacaaaagcagacgcagctttattgaggaaaagaaaatgccttaatttactaagttacaaagctaatgtcaccactaactgctttactcgctgcttgcaaaacaaattcaaaaagttagcattataattagaataggatttaaaccccccggtcatttcctgctcaataccattcccctgaacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgctttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgttttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 6 - DNA, 16 bp, Artificial Sequence
  rep binding site (RBS)
gctcgctcgctcgctc

SEQ ID NO: 7 - DNA, 19 bp, Artificial Sequence
  rep forward primer
cacgtgcatgtggaagtag

SEQ ID NO: 8 - DNA, 19 bp, Artificial Sequence
  rep reverse primer
cgactttctgacggaatgg

SEQ ID NO: 9 - DNA, 20 bp, Artificial Sequence
  cap forward primer
tactgagggaccatgaagac

SEQ ID NO: 10 - DNA, 20 bp, Artificial Sequence
  cap reverse primer
gtttacggactcggagtatc